(19) United States
(12) Patent Application Publication
(10) Pub. No.: US 2025/0136867 A1
(43) Pub. Date: May 1, 2025

(54) POLYMERIZABLE COMPOUNDS FOR ORTHODONTIC DEVICES

(71) Applicant: Align Technology, Inc., San Jose, CA (US)

(72) Inventors: Kejia Yang, Santa Clara, CA (US); Michael Christopher Cole, San Jose, CA (US); Xiance Wang, Santa Clara, CA (US); Umesh Upendra Choudhary, Scotts Valley, CA (US)

(21) Appl. No.: 18/933,844

(22) Filed: Oct. 31, 2024

Related U.S. Application Data
(60) Provisional application No. 63/594,695, filed on Oct. 31, 2023.

(51) Int. Cl. C09K 19/20; C09K 19/32; C09K 19/34

(52) U.S. Cl. CPC C09K 19/2014; C09K 19/3405; C09K 19/3483; C09K 19/3491; C09K 2019/323; C09K 2019/3425

(57) ABSTRACT

The present disclosure provides curable compositions comprising one or more of polymerizable liquid crystalline compounds or polymerizable polyesters with disrupted crystallinity, as well as polymeric materials formed from the curable compositions. Further provided herein are methods of producing the compositions and using the same for the fabrication of medical devices, such as orthodontic appliances.

### INCORPORATION BY REFERENCE

All publications, patents, and patent applications mentioned in this disclosure are herein incorporated by reference to the same extent as if each individual publication, patent, or patent application was specifically and individually indicated to be incorporated by reference in its entirety. This application claims priority to U.S. Provisional Patent Application No. 63/594,695, filed Oct. 31, 2023, entitled “POLYMERIZABLE COMPOUNDS FOR ORTHODONTIC DEVICES,” the contents of which are hereby incorporated by reference as if set forth fully herein.

### BACKGROUND

Orthodontic devices are subject to stringent requirements for strength, flexibility, durability, size, weight, and appearance. While strong devices are often required to affect desired treatment outcomes, such as tooth alignment, practical limitations concerning comfort, appearance, and patient compliance often require these devices be small, lightweight, and transparent or neutral in appearance. Furthermore, for many dental applications, materials must be compatible with high resolution printing methods. As few materials offer this combination of features, new materials are needed to enable current and emerging dental treatment technologies.

### BRIEF SUMMARY

The present disclosure provides curable compositions comprising one or more of polymerizable liquid crystalline compounds or polymerizable polyesters with disrupted crystallinity usable by a range of 3D-printing methods and configured for a variety of practical applications. Further provided herein are polymeric materials formed from the curable compositions as well as methods of producing the compositions and using the same for the fabrication of medical devices, such as orthodontic appliances.

In one aspect, provided is a polymerizable compound having the following structure (III):

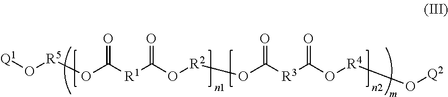

wherein R¹ and R³ are, at each occurrence, independently an arylene or heteroarylene group; R² is, at each occurrence, independently a linear alkylene or arylene bis(alkylene ester) group; R⁴ is, at each occurrence, independently a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group; R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group; Q¹ and Q² are each independently an end-capping moiety comprising one or more reactive functional groups; m is an integer of one or greater; and n1 and n2 are, at each occurrence, independently an integer of one or greater for each integral value of m, provided that if R¹ and R³ are the same, then R² and R⁴ are different, and if R² and R⁴ are the same, then R¹ and R³ are different. In some embodiments, R¹ and R³ are unsubstituted C₆-C₂₀ arylene or C₆-C₂₀ arylene substituted with one or more C₁-C₆ alkyl or C₁-C₆ alkoxy. In some embodiments, R¹ and R³ are phenylene, biphenylene or naphthalene, wherein the phenylene, biphenylene or naphthalene is optionally substituted with one or more C₁-C₆ alkyl or C₁-C₆ alkoxy. In some embodiments, R¹ and R³ are furanylene, thienylene, pyrrolyene, imidazolyene, pyrazolyene, oxazolyene or thiazolyene.

In some embodiments, R¹ and R³ have one of the following structures:

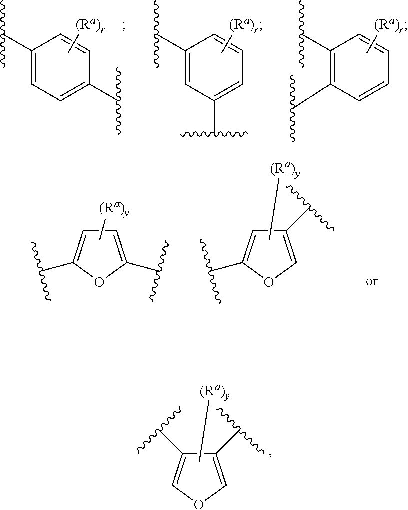

wherein R^{a} is, at each occurrence, C₁-C₆ alkyl or C₁-C₆ alkoxy; r is an integer from 0 to 4; and y is an integer from 0 to 2.

In some embodiments, R¹ and R³ have one of the following structures:

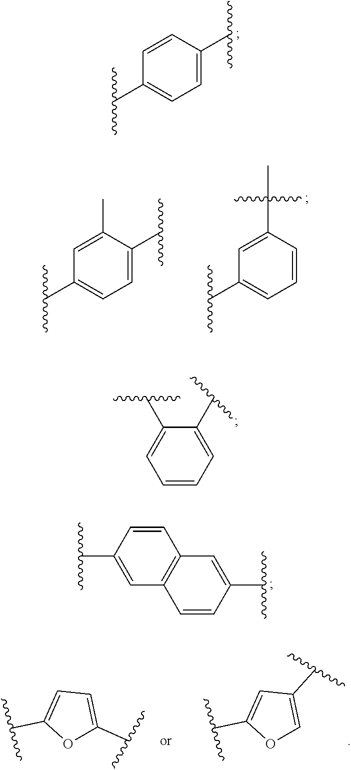

In some embodiments, R¹ is an unsubstituted 1,4-phenylene group and R³ is a substituted or unsubstituted phenylene group, and the compound has the following structure (IA):

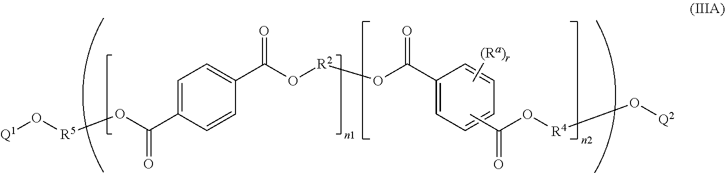

wherein R^{a} is, at each occurrence, C₁-C₆ alkyl or C₁-C₆ alkoxy; and r is, at each occurrence, an integer from 0 to 4, provided that if r is 0 and both phenylenes are unsubstituted 1,4-phenylene groups, then R² and R⁴ are different.

In some embodiments, R³ is a substituted or unsubstituted 1,4-phenylene group, and the compound has the following structure (IIIB):

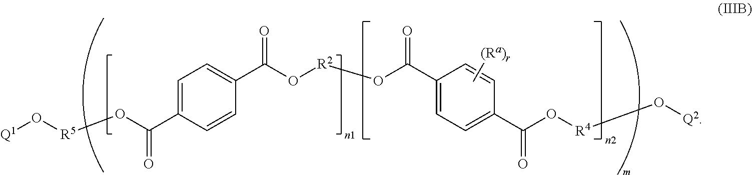

In some embodiments, R³ is an unsubstituted 1,2-phenylene or 1,3-phenylene group, and the compound has the following structure (IIIC) or (IIID):

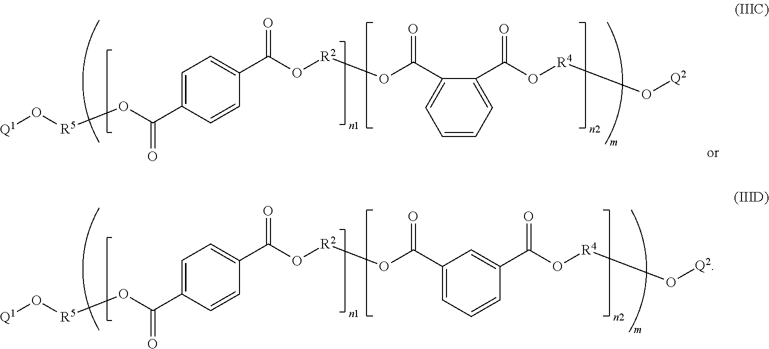

In some embodiments, R⁴ is a linear or branched C₁-C₁₂ alkylene. In some embodiments, R⁴ is ethylene, propylene, isopropylene, n-butylene, isobutylene, tert-butylene, pentylene, isopentylene, 3-methylpentylene, hexylene, octylene, nonylene, decylene or dodecylene. In some embodiments, R⁴ is a linear or branched C₂-C₁₂ heteroalkylene.

In some embodiments, R⁴ has one of the following structures:

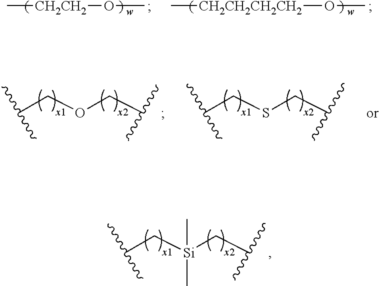

wherein w is an integer from 1 to 500; and x1 and x2 are independently an integer from 1 to 6.

In some embodiments, R⁴ is a phenylene bis(alkylene ester) or phenylene bis(heteroalkylene ester)group, and the compound has the following structure (IIIE):

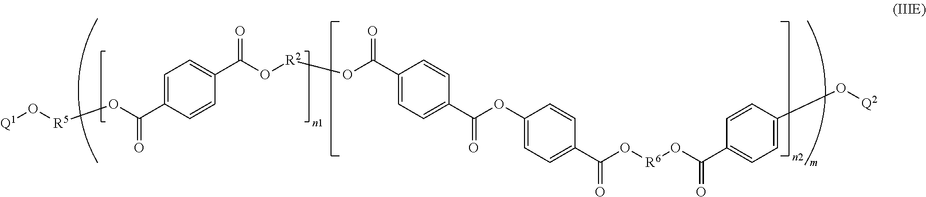

wherein R⁶ is, at each occurrence, a linear or branched C₁-C₁₂ alkylene, C₃-C₁₈ cycloalkylene or linear or branched C₂-C₁₂ heteroalkylene group.

In some embodiments, R⁴ is a bis(carboxycyclohexylene) alkylene or bis(carboxycyclohexylene) heteroalkene group, and the compound has the following structure (IIIF):

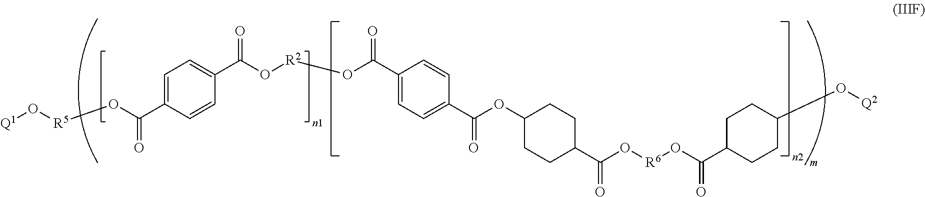

wherein R⁶ is, at each occurrence, a linear or branched C₁-C₁₂ alkylene, C₃-C₁₈ cycloalkylene, or linear or branched C₂-C₁₂ heteroalkylene group

In some embodiments R⁶ has one of the following structures:

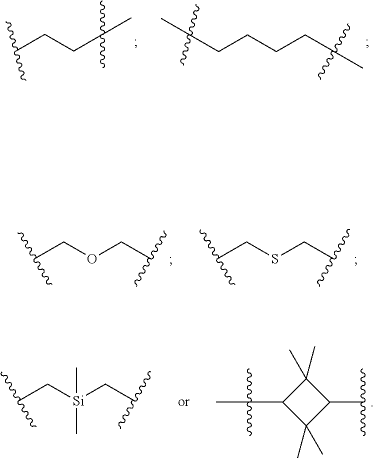

In some embodiments, n1 and n2 are each independently an integer from 1 to 50.

In some embodiments, the compound has the following structure (IIIG):

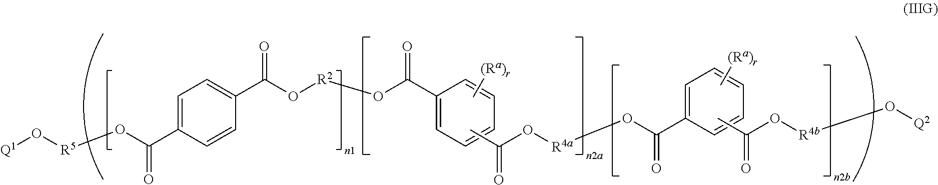

wherein R^{4a} and R^{4b}, at each occurrence, a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group, provided that R^{4a} and R^{4b} are different; R^{a} is, at each occurrence, C₁-C₆ alkyl or C₁-C₆ alkoxy; r is, at each occurrence, an integer from 0 to 4; m is an integer of one or greater; and n1, n2a and n2b are, at each occurrence, independently an integer of one or greater for each integral value of m.

In some embodiments, R^{4a} and R^{4b} are each a linear or branched C₁-C₁₂ alkylene. In some embodiments, R^{4a} and R^{4b} are independently ethylene, propylene, isopropylene, n-butylene, isobutylene, tert-butylene, pentylene, isopentylene, 3-methylpentylene, hexylene, octylene, nonylene, decylene or dodecylene. In some embodiments, R^{4a} and R^{4b} are each a linear or branched C₂-C₁₂ heteroalkylene.

In some embodiments, R^{4a} and R^{4b} independently have one of the following structures:

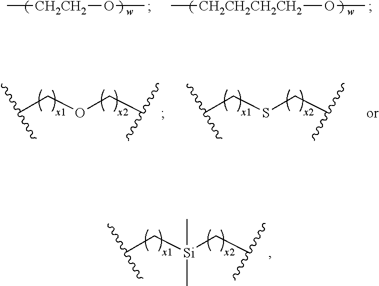

wherein w is an integer from 1 to 500; and x1 and x2 are independently an integer from 1 to 6.

In some embodiments, n1, n2a and n2b are each independently an integer from 1 to 50.

In some embodiments, m is an integer from 1-50.

In some embodiments, R² is ethylene, propylene, butylene, pentylene, hexylene, octylene, nonylene, decylene or dodecylene.

In some embodiments, R⁵ is the same as R². In some embodiments, R⁵ is the same as R⁴. In some embodiments, R⁵ is ethylene, propylene, butylene, pentylene or hexylene.

In some embodiments, Q¹ and Q² independently comprise an acrylate, methacrylate, vinyl acrylate, vinyl methacrylate, allyl ether, silene, alkyne, alkene, vinyl ether, maleimide, fumarate, maleate, itaconate, vinyl ester, vinyl ketone, epoxide, oxetane, cyclic ester or styrenyl moiety. In some embodiments, Q¹ and Q² independently have one of the following structures:

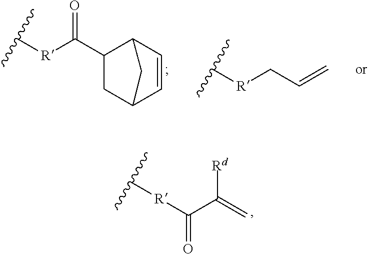

wherein R′ is an optional C₁-C₆ heteroalkylene linker; and R^{d} is H, halogen, or C₁-C₃ alkyl.

In some embodiments, Q¹ and Q² independently have one of the following structures:

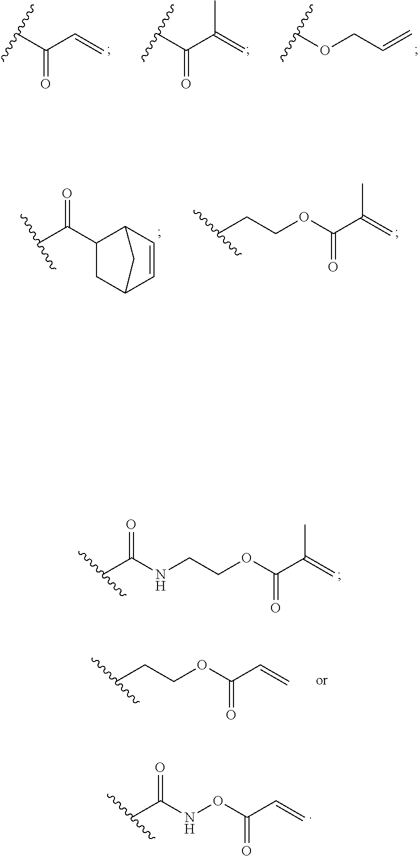

In some embodiments, Q¹ and Q² independently have one of the following structures:

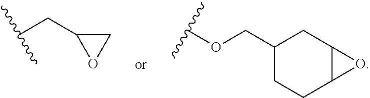 [SMILES: *CC1CO1 ; *OCC1CCC2OC2C1]

In some embodiments, the compound of structure (III) is a compound selected from Table.

In another aspect, provided is a curable composition comprising an initiator and a polymerizable compound of structure (III).

In still another aspect, provided is a curable composition comprising an initiator and a polymerizable compound having the following structure (II):

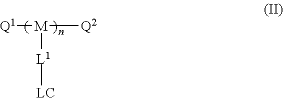

wherein M is an atom or group of atoms of a polymer backbone; LC is a liquid crystalline moiety; L¹ is an optional alkylene or heteroalkylene linker covalently bound a liquid crystalline moiety to the polymer backbone; Q¹ and Q² are each independently an end-capping moiety comprising one or more reactive functional groups; and n is an integer of one or greater.

In some embodiments, the polymer backbone comprises repeating units derived from one or more monomers selected from acrylates, acrylic acids, siloxanes, hydroxystyrenes, methacrylates, vinyl esters, maleic esters, methacrylonitriles and methacrylamides. In some embodiments, the polymer backbone comprises polyhydroxystyrene, polyacrylate, polymethacrylate, substituted polyethylene, poly(vinyl ester), or polysiloxane backbone.

In some embodiments, the polymerizable compound of structure (II) has the following structure (IIA):

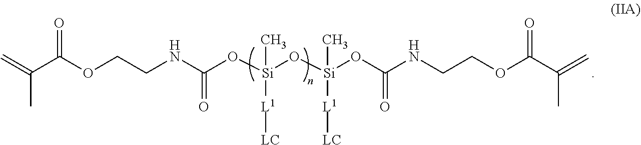

In some embodiments, the polymerizable compound of structure (II) has the following structure (IIB):

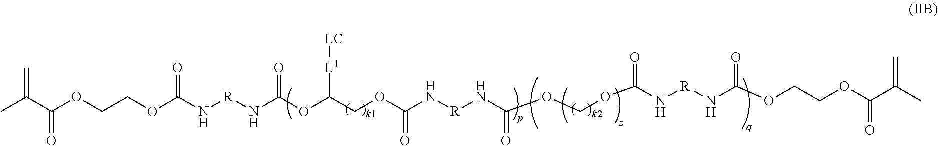

wherein R is a cycloalkylene, arylene, cycloalkylenedialkylene or arylenedialkylene group; k1 and k2 are independently integer from 1 to 6; p is an integer of one or greater; and q is an integer of zero or greater. In some embodiments, R is a cycloalkylene, arylene, cycloalkylenedialkylene or arylenedialkylene group.

In some embodiments, R is cyclohexanedimethylene or cyclohexanemethylene. In some embodiments, R has one of the following structures:

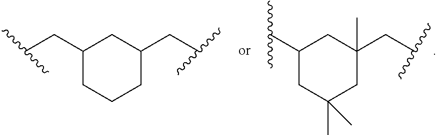 [SMILES: *CC1CCCC(C*)C1 ; *CC1(C)CC(*)CC(C)(C)C1]

In some embodiments, p is an integer from 1 to 300. In some embodiments, q is an integer from 1 to 300. In some embodiments, q is 0, and the polymerizable compound of structure (IIB) has the following structure (IIB-1):

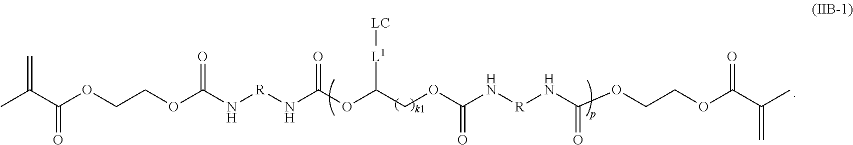

In some embodiments, L¹ is a heteroalkylene linker. In some embodiments, L¹ has one of the following structures:

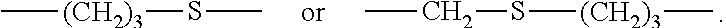

In some embodiments, the LC has the following structure:

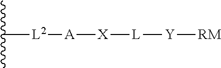

wherein L² is an alkylene linker; A is an optional cycloalkylene or arylene linker; X is an optional ester group; L is an optional alkylene or heteroalkylene linker; Y is an optional amide, ether, ester or urethane group; an RM is a rigid moiety comprising one or more aromatic or cycloaliphatic rings. In some embodiments, L² is ethylene, propylene, butylene, pentylene or hexylene.

In some embodiments, A has one of the following structures:

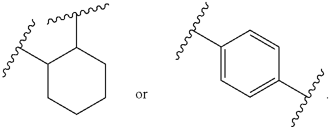 [SMILES: *C1CCCCC1* ; *c1ccc(*)cc1]

In some embodiments, L is C₁-C₈ alkylene or C₃-C₈ heteroalkylene. In some embodiments, L has one of the following structures:

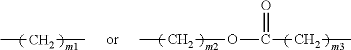

wherein m1, m2 and m3 are each independently an integer from 1 to 12.

In some embodiments, m1 is an integer from 2 to 8, and m2 and m3 are each independently 2.

In some embodiments, RM comprises a cycloalkyl, aryl, heteroaryl or alkylenearyl group.

In some embodiments, RM has one of the following structures:

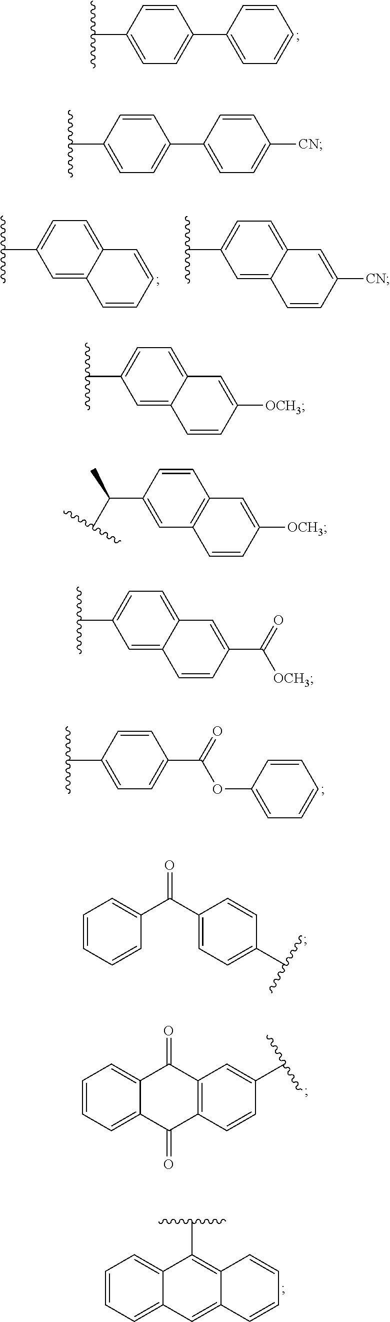

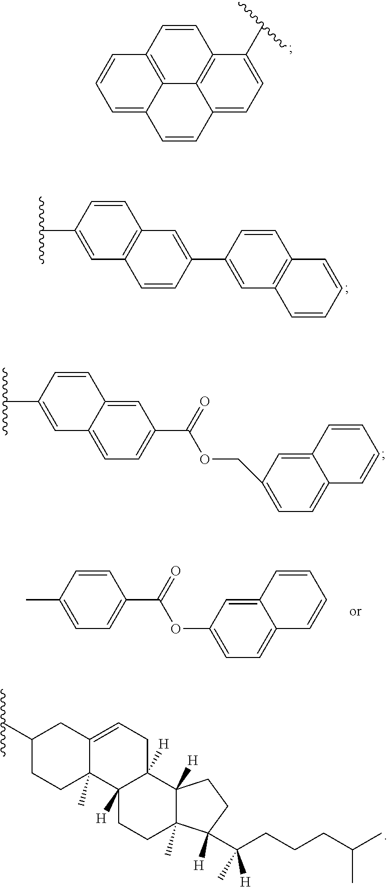 [SMILES: *c1ccc2ccc3cccc4ccc1c2c34 ; *c1ccc2cc(-c3ccc4ccccc4c3)ccc2c1 ; *c1ccc2cc(C(=O)OCc3ccc4ccccc4c3)ccc2c1 ; Cc1ccc(C(=O)Oc2ccc3ccccc3c2)cc1 ; *C1CC[C@@]2(C)C(=CC[C@H]3[C@@H]4CC[C@H]([C@@H](C)CCCC(C)C)[C@@]4(C)CC[C@@H]32)C1]

In some embodiments, Q¹ and Q² independently comprise an acrylate, methacrylate, vinyl acrylate, vinyl methacrylate, allyl ether, silene, alkyne, alkene, vinyl ether, maleimide, fumarate, maleate, itaconate, vinyl ester, vinyl ketone, epoxide, oxetane, cyclic ester or styrenyl moiety. In some embodiments, Q¹ and Q² have one of the following structures:

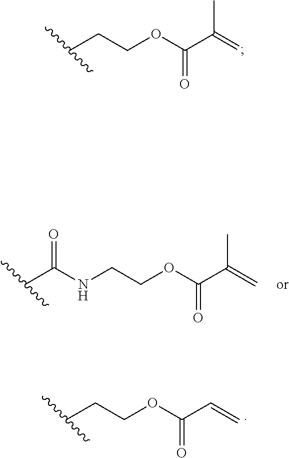

In some embodiments, the polymerizable compound of structure (IIA) has the following structure:

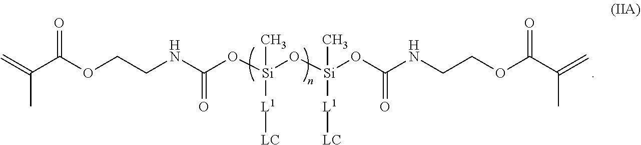

In some embodiments, the compound of structure (IIB) has one of the following structures:

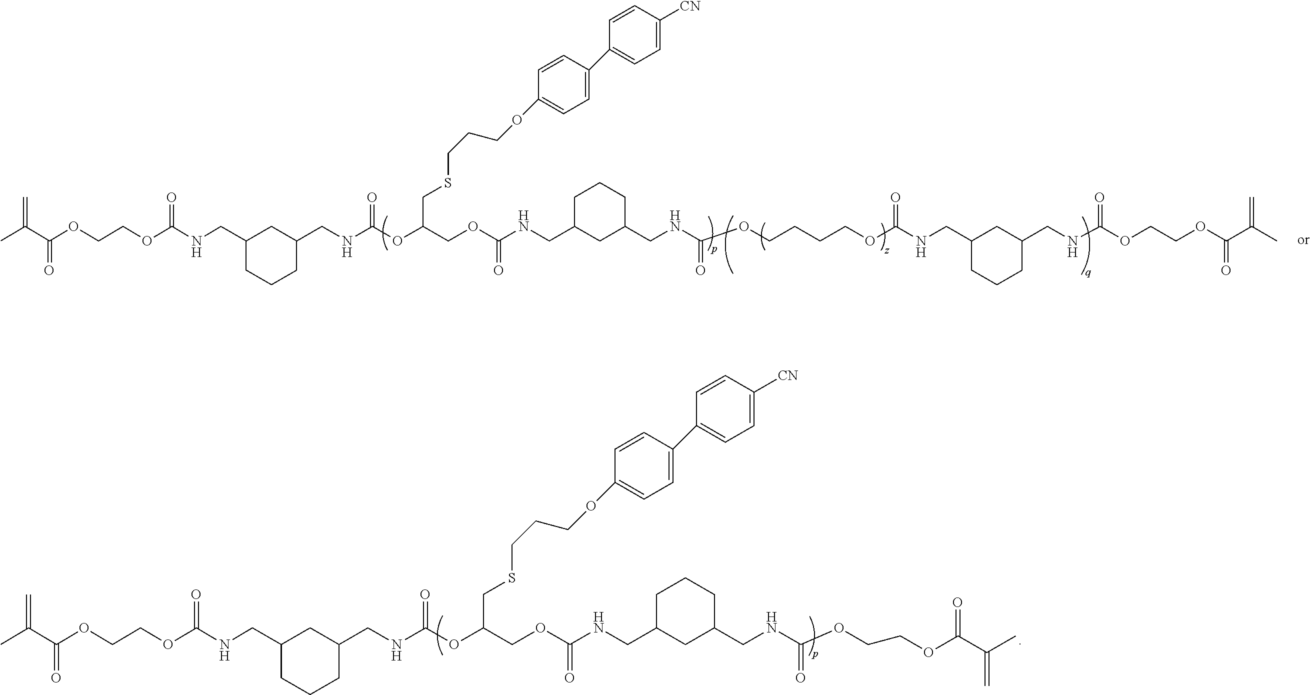

In some embodiments, a curable composition for preparing an orthodontic appliance comprising an initiator; and a reactive diluent comprising a polymerizable liquid crystalline compound having the following structure (I):

G-A-X-L-Y-RM, (I)

wherein G is a polymerizable group; A is an optional cycloalkylene or arylene linker; X is an optional ester group; L is an optional alkylene or heteroalkylene linker; Y is an optional amide, ether, ester or urethane group; and RM is a rigid moiety comprising one or more aromatic or cycloaliphatic rings.

In some embodiments, G comprises an acrylate, methacrylate, vinyl acrylate, vinyl methacrylate, allyl ether, silene, alkyne, alkene, vinyl ether, maleimide, fumarate, maleate, itaconate, vinyl ester, vinyl ketone, epoxide, oxetane, cyclic ester or styrenyl moiety.

In some embodiments, G has one of the following structures:

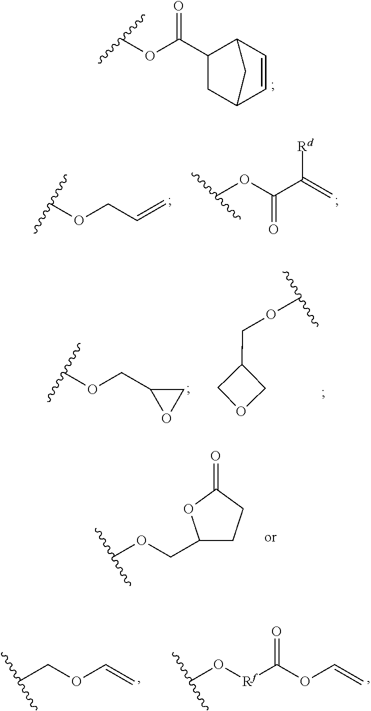

wherein R^{d} is H, halogen, or C₁-C₃ alkyl; and R^{f} is C₁-C₆ alkylene.

In some embodiments, G has one of the following structures:

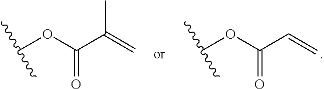

In some embodiments, A has one of the following structures:

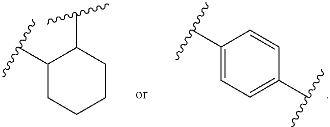 [SMILES: *C1CCCCC1* ; *c1ccc(*)cc1]

In some embodiments, L is C₁-C₈ alkylene or C₃-C₈ heteroalkylene.

In some embodiments, L has one of the following structures:

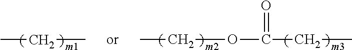

wherein m1, m2 and m3 are each independently an integer from 1 to 12.

In some embodiments, m1 is an integer from 2 to 8, and m2 and m3 are each independently 2.

In some embodiments, RM comprises a cycloalkyl, aryl, heteroaryl or alkylenearyl group.

In some embodiments, RM has one of the following structures:

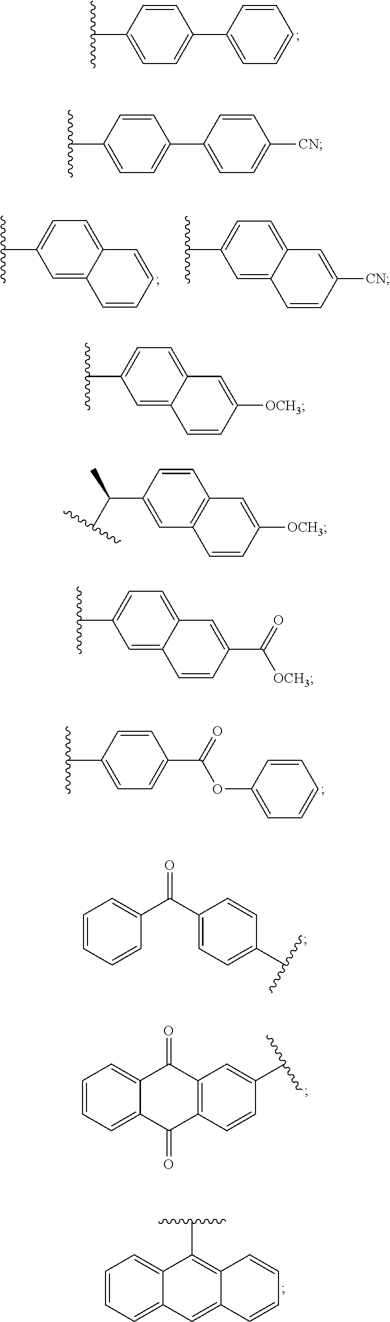

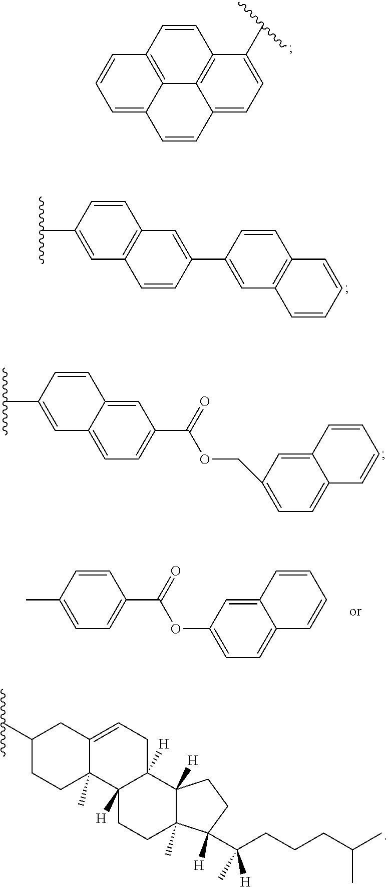 [SMILES: *c1ccc2ccc3cccc4ccc1c2c34 ; *c1ccc2cc(-c3ccc4ccccc4c3)ccc2c1 ; *c1ccc2cc(C(=O)OCc3ccc4ccccc4c3)ccc2c1 ; Cc1ccc(C(=O)Oc2ccc3ccccc3c2)cc1 ; *C1CC[C@@]2(C)C(=CC[C@H]3[C@@H]4CC[C@H]([C@@H](C)CCCC(C)C)[C@@]4(C)CC[C@@H]32)C1]

In some embodiments, the curable composition further comprises a reactive diluent.

In some embodiments, the reactive diluent is syringyl methacrylate (SMA) or a polymerizable liquid crystalline compound of structure (I).

In some embodiments, the initiator comprises a photoinitiator.

In some embodiments, photoinitiator comprises 2,4,6-trimethylbenzoyl diphenyl phosphine oxide (TPO) or ethyl(2,4,6-trimethylbenzoyl)phenyl phosphinate (TPO-L).

In some embodiments, the curable composition comprises 0.01-10 wt % of initiator.

In some embodiments, the curable composition further comprises one or more reagents selected from the group consisting of a crosslinking modifier, a glass transition temperature modifier, a toughness modifier, a polymerization catalyst, a polymerization inhibitor, a light blocker, a plasticizer, a surface energy modifier, a pigment, a dye, a filler, a biologically significant chemical, a solvent and combinations thereof. In some embodiments, the curable composition is capable of being 3D printed at a printing temperature greater than 25° C. In some embodiments, the printing temperature is at least 30° C., 40° C., 50° C., 60° C., 80° C. or 100° C. In some embodiments, the curable composition has a viscosity from 30 cP to 50,000 cP at a printing temperature. In some embodiments, the printing temperature is from 25° C. to 150° C. In some embodiments, the photocurable composition comprises less than 20 wt % hydrogen bonding units. In some embodiments, the photocurable composition is a liquid at a temperature from about 40° C. to about 100° C. In some embodiments, the curable composition is a liquid at a temperature of above about 40° C. with a viscosity less than about 20 Pa·s. In some embodiments, the curable composition is a liquid at a temperature of above about 40° C. with a viscosity less than about 1 Pa·s. In some embodiments, at least a portion of the curable composition melts at a temperature between about 60° C. and about 100° C.

In still another aspect, provided is a polymeric material formed from any one of the curable compositions disclosed herein. In some embodiments, the polymeric material has one or more of the following characteristics: (A) a storage modulus greater than or equal to 200 MPa at 37° C.; (B) a flexural stress and/or flexural modulus of greater than or equal to 1.5 MPa remaining after 24 hours in a wet environment at 37° C.; (C) an elongation at break greater than or equal to 5% before and after 24 hours in a wet environment at 37° C.; (D) a water uptake of less than 25 wt % when measured after 24 hours in a wet environment at 37° C.; (E) transmission of at least 30% of visible light through the polymeric material after 24 hours in a wet environment at 37° C.; and (F) comprises a plurality of polymeric phases, wherein at least one polymeric phase of the one or more polymeric phases has a Tg of at least 60° C., 80° C., 90° C., 100° C., or at least 110° C. In some embodiments, the polymeric material is characterized by a water uptake of less than 20 wt %, less than 15 wt %, less than 10 wt %, less than 5 wt %, less than 4 wt %, less than 3 wt %, less than 2 wt %, less than 1 wt %, less than 0.5 wt %, less than 0.25 wt %, or less than 0.1 wt % when measured after 24 hours in a wet environment at 37° C. In some embodiments, the polymeric material has greater than 60% conversion of double bonds to single bonds compared to the curable composition, as measured by FTIR. In some embodiments, the polymeric material has greater than 60% conversion of double bonds to single bonds compared to the curable composition, as measured by FTIR. In some embodiments, the polymeric material has an ultimate tensile strength from 10 MPa to 100 MPa, from 15 MPa to 80 MPa, from 20 MPa to 60 MPa, from 10 MPa to 50 MPa, from 10 MPa to 45 MPa, from 25 MPa to 40 MPa, from 30 MPa to 45 MPa, or from 30 MPa to 40 MPa after 24 hours in a wet environment at 37° C. In some embodiments, the polymeric material is characterized by an elongation at break greater than 10%, an elongation at break greater than 20%, an elongation at break greater than 30%, an elongation at break of 5% to 250%, an elongation at break of 20% to 250%, or an elongation at break value between 40% and 250% before and after 24 hours in a wet environment at 37° C. In some embodiments, the polymeric material is characterized by a storage modulus of 0.1 MPa to 4000 MPa, a storage modulus of 300 MPa to 3000 MPa, or a storage modulus of 750 MPa to 3000 MPa after 24 hours in a wet environment at 37° C. In some embodiments, the polymeric material has a flexural stress and/or flexural modulus of 400 MPa or more, 300 MPa or more, 200 MPa or more, 180 MPa or more, 160 MPa or more, 120 MPa or more, 100 MPa or more, 80 MPa or more, 70 MPa or more, 60 MPa or more, after 24 hours in a wet environment at 37° C. In some embodiments, at least 40%, 50%, 60%, or 70% of visible light passes through the polymeric material after 24 hours in a wet environment at 37° C. In some embodiments, the polymeric material is biocompatible, bioinert, or a combination thereof.

In still another aspect, provided is a polymeric film comprising a polymeric material disclosed herein. In some embodiments, the polymeric film has a thickness of at least 100 μm and not more than 3 mm.

In still another aspect, provided is an orthodontic appliance comprising a polymeric material or a polymeric film disclosed herein. In some embodiments, the orthodontic appliance is an aligner, expander or spacer.

In still another aspect, provided is a method of forming a polymeric material disclosed herein comprising: providing a curable composition disclosed herein; exposing the curable composition to a light source; and curing the curable composition to form the polymeric material. In some embodiments, the light source is an ultraviolet (UV) or visible light source. In some embodiments, the method further comprises inducing phase separation during photo-curing. In some embodiments, inducing phase separation comprises generating one or more polymeric phases in the polymeric material during photo-curing. In some embodiments, at least one polymeric phase of the one or more polymeric phases is an amorphous phase having a glass transition temperature (Tg) of at least 60° C., 80° C., 90° C., 100° C., or at least 110° C. In some embodiments, at least 25%, 50%, or 75% of polymeric phases generated during photo-curing are amorphous phases having a glass transition temperature (Tg) of at least 60° C., 80° C., 90° C., 100° C., or at least 110° C. In some embodiments, at least one polymeric phase of the one or more polymeric phases is a crystalline phase comprising a crystalline polymeric material. In some embodiments, the crystalline polymeric material has a melting point of at least 60° C., 80° C., 90° C., 100° C., or at least 110° C. In some embodiments, at least one polymeric phase of the one or more polymeric phases is 3-dimensional and has at least one dimension with a length of less than 1000 μm, less than 500 μm, less than 250 μm, or less than 200 μm. In some embodiments, further comprises fabricating an orthodontic appliance with the polymeric material.

In still another aspect, provided is a method for preparing an article by an additive manufacturing process comprising: providing a curable composition disclosed herein; heating the curable composition to a processing temperature; exposing the curable composition to radiation; curing the curable composition layer-by-layer based on a predefined design, thereby polymerizing and crosslinking the polymerizable compound to form a polymeric material; and fabricating the article with the polymeric material. In some embodiments, the processing temperature is from about 50° C. to about 120° C. In some embodiments, the processing temperature is from about 90° C. to about 110° C., from about 100° C. to about 120° C., from about 105° C. to about 115° C., or from about 108° C. to about 110° C. In some embodiments, the additive manufacturing process is a 3D printing process. In some embodiments, the article is a medical device. In some embodiments, the medical device is an orthodontic appliance.

In still another aspect, provided is a method of repositioning a patient's teeth comprising: generating a treatment plan for the patient, the plan comprising a plurality of intermediate tooth arrangements for moving teeth along a treatment path from an initial tooth arrangement toward a final tooth arrangement; producing an orthodontic appliance according to any of claims 86-87, or an orthodontic appliance comprising the polymeric material disclosed herein; and moving on-track, with the orthodontic appliance, at least one of the patient's teeth toward an intermediate tooth arrangement or the final tooth arrangement. In some embodiments, producing the orthodontic appliance comprises 3D printing of the orthodontic appliance. In some embodiments, the method further comprises tracking progression of the patient's teeth along the treatment path after administration of the orthodontic appliance to the patient, the tracking comprising comparing a current arrangement of the patient's teeth to a planned arrangement of the patient's teeth. In some embodiments, greater than 60% of the patient's teeth are on track with the treatment plan after 2 weeks of treatment. In some embodiments, the orthodontic appliance has a retained repositioning force to the at least one of the patient's teeth after 2 days that is at least 10%, at least 20%, at least 30%, at least 40%, at least 50%, at least 60%, or at least 70% of repositioning force initially provided to the at least one of the patient's teeth.

### BRIEF DESCRIPTION OF THE SEVERAL VIEWS OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A illustrates a tooth repositioning appliance, in accordance with some embodiments.

FIG. 1B illustrates a tooth repositioning system, in accordance with some embodiments.

FIG. 1C illustrates a method of orthodontic treatment using a plurality of appliances, in accordance with some embodiments.

FIG. 2 illustrates a method for designing an orthodontic appliance, in accordance with some embodiments.

FIG. 3 illustrates a method for digitally planning an orthodontic treatment, in accordance with embodiments.

FIG. 4 shows generating and administering treatment, in accordance with some embodiments.

FIG. 5 illustrates the lateral dimensions and vertical dimension as used herein.

FIG. 6 shows a schematic configuration of a high temperature additive manufacturing device used for curing curable compositions of the present disclosure by using a 3D printing process.

FIG. 7 illustrates a polymerizable compound with liquid crystalline side chains, in accordance with some embodiments.

FIG. 8 shows DMA results of a polymerizable compound with disrupted crystallinity prepared according to Example 2.

FIG. 9 shows the tensile stress of a polymeric material obtained from a curable composition according to Example 9 as a function of tensile strain.

FIG. 10 shows the effect of relative amounts of butylene and pentylene terephthalates on the degree of crystallinity.

FIG. 11 shows the effect of relative amounts of pentylene and hexylene terephthalates on the degree of crystallinity and melting temperature.

### DETAILED DESCRIPTION

In the following description, certain specific details are set forth to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Number ranges are to be understood as inclusive, i.e., including the indicated lower and upper limits. Furthermore, the term “about”, as used herein, and unless clearly indicated otherwise, generally refers to and encompasses plus or minus 10% of the indicated numerical value(s). For example, “about 10%” may indicate a range of 9% to 11%, and “about 1” may include the range 0.9-1.1.

As used herein, the term “polymer” generally refers to a molecule composed of repeating structural units connected by covalent chemical bonds and characterized by a substantial number of repeating units (e.g., equal to or greater than 20 repeating units and often equal to or greater than 100 repeating units and often equal to or greater than 200 repeating units) and a number average molecular weight greater than or equal to 5,000 Daltons (Da) or 5 kDa, such as greater than or equal to 10 kDa, 15 kDa, 20 kDa, 30 kDa, 40 kDa, 50 kDa, or 100 kDa. Polymers are commonly the polymerization product of one or more monomer precursors. The term polymer includes homopolymers, i.e., polymers consisting essentially of a single repeating monomer species. The term polymer also includes copolymers which are formed when two or more different types of monomers are linked in the same polymer. Copolymers may comprise two or more monomer subunits, and include random, block, alternating, segmented, grafted, tapered and other copolymers. The term “cross-linked polymers” generally refers to polymers having one or multiple links between at least two polymer chains, which can result from multivalent monomers forming cross-linking sites upon polymerization.

As used herein, the term “oligomer” generally refers to a molecule composed of repeating structural units connected by covalent chemical bonds and characterized by a number of repeating units less than that of a polymer (e.g., equal to or less than 10 repeating units) and a lower molecular weight than polymers (e.g., less than 5,000 Da or 2,000 Da). In some cases, oligomers may be the polymerization product of one or more monomer precursors. In an embodiment, an oligomer or a monomer cannot be considered a polymer in its own right.

As used herein, the terms “telechelic polymer” and “telechelic oligomer” generally refer to a polymer or oligomer that is capable of entering, through reactive groups, into further polymerization.

As used herein, the term “reactive diluent” generally refers to a substance which reduces the viscosity of another substance, such as a monomer or curable resin. A reactive diluent may become part of another substance, such as a polymer obtained by a polymerization process. In some examples, a reactive diluent is a curable monomer which, when mixed with a curable resin, reduces the viscosity of the resultant formulation and is incorporated into the polymer that results from polymerization of the formulation.

Oligomer and polymer mixtures can be characterized and differentiated from other mixtures of oligomers and polymers by measurements of molecular weight and molecular weight distributions.

The average molecular weight (M) is the average number of repeating units n times the molecular weight or molar mass (Mᵢ) of the repeating unit. The number-average molecular weight (Mₙ) is the arithmetic mean, representing the total weight of the molecules present divided by the total number of molecules. The weight-average molecular weight (M_{w}) is the sum of the products of the molar mass of each fraction multiplied by its weight fraction. A ratio of the weight-average molecular weight to the number-average molecular weight (M_{w}/Mₙ) defines polydispersity index of a polymer.

Photoinitiators described in the present disclosure can include those that can be activated with light and initiate polymerization of the polymerizable components of the formulation. In some embodiments, a photoinitiator may be a free radical initiator that can produce radical species and/or promote radical reactions upon exposure to radiation (e.g., UV or visible light). In some other embodiments, a photoinitiator may be an ionic initiator that can produce ionic species upon exposure to radiation (e.g., UV or visible light). In some embodiments, the ionic initiator is a cationic initiator. In some embodiments, the ionic initiator is an anionic initiator.

Thermal initiators described in the present disclosure can include those that can be activated with heat and initiate polymerization of the polymerizable components of the formulation. A “thermal initiator”, as used herein, may generally refer to a compound that can produce radical species and/or promote radical reactions upon exposure to heat.

The term “biocompatible,” as used herein, refers to a material that does not elicit an immunological rejection or detrimental effect, referred herein as an adverse immune response, when it is disposed within an in-vivo biological environment. For example, in embodiments a biological marker indicative of an immune response changes less than 10%, or less than 20%, or less than 25%, or less than 40%, or less than 50% from a baseline value when a human or animal is exposed to or in contact with the biocompatible material. Alternatively, immune response may be determined histologically, wherein localized immune response is assessed by visually assessing markers, including immune cells or markers that are involved in the immune response pathway, in and adjacent to the material. In an aspect, a biocompatible material or device does not observably change immune response as determined histologically. In some embodiments, the disclosure provides biocompatible devices configured for long-term use, such as on the order of weeks to months, without invoking an adverse immune response. Biological effects may be initially evaluated by measurement of cytotoxicity, sensitization, irritation and intracutaneous reactivity, acute systemic toxicity, pyrogenicity, subacute/subchronic toxicity and/or implantation. Biological tests for supplemental evaluation include testing for chronic toxicity.

“Bioinert” refers to a material that does not elicit an immune response from a human or animal when it is disposed within an in-vivo biological environment. For example, a biological marker indicative of an immune response remains substantially constant (plus or minus 5% of a baseline value) when a human or animal is exposed to or in contact with the bioinert material. In some embodiments, the disclosure provides bioinert devices.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

As used herein, the term “group” may refer to a functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, the term “substituted” refers to a compound (e.g., an alkyl chain) wherein a hydrogen is replaced by another functional group or atom, as described herein.

As used herein, a broken line in a chemical structure can be used to indicate a bond to the rest of the molecule. For example, [symbol] in

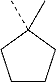

is used to designate the 1-position as the point of attachment of 1-methylcyclopentate to the rest of the molecule. Alternatively, [symbol] in, e.g.,

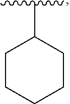

can be used to indicate that the given moiety, the cyclohexyl moiety in this example, is attached to a molecule via the bond that is “capped” with the wavy line.

As used herein, a “linker” refers to a contiguous chain of at least one atom, such as carbon, oxygen, nitrogen, sulfur, phosphorous, and combinations thereof, which connects a portion of a molecule to another portion of the same molecule or to a different molecule, moiety or solid support (e.g., microparticle). Linkers may connect the molecule via a covalent bond or other means, such as ionic or hydrogen bond interactions. In some embodiments, the linker is a heteroatomic linker (e.g., comprising 1-10 Si, N, O, P, or S atoms), a heteroalkylene (e.g., comprising 1-10 Si, N, O, P, or S atoms and an alkylene chain) or an alkylene linker (e.g., comprising 1-12 carbon atoms). In some embodiments, the linker may contain an ether (—O—), ester (—OC(═O)—), or carbonate (—OC(═O)O—) linkage.

“Aliphatic” or “aliphatic group” as used herein means a straight-chain or branched C₁₋₁₈ hydrocarbon chain that is completely saturated or that contains one or more units of unsaturation, or a monocyclic C₃₋₁₈ hydrocarbon or bicyclic C₈₋₁₈ hydrocarbon that is completely saturated or that contains one or more units of unsaturation, but which is not aromatic (also referred to herein as “cycloalkyl”), that has a single point of attachment to the rest of the molecule where in any individual ring in said bicyclic ring system has 3-7 members. For example, suitable aliphatic groups include, but are not limited to, linear or branched alkyl, alkenyl, alkynyl groups and hybrids thereof such as (cycloalkyl)alkyl, (cycloalkenyl)alkyl or (cycloalkyl)alkenyl.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 3-methylpentyl, 1-dimethylethyl (t-butyl), 3-methylhexyl, 2-methylhexyl and the like. The number of carbons referred to relates to the carbon backbone and carbon branching but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂-C₈ alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching but does not include carbon atoms belonging to any substituents. Unless stated otherwise specifically in the specification, an alkenyl group is optionally substituted.

“Alkynyl” refers to unsaturated straight or branched hydrocarbon radical, having 2 to 12 carbon atoms (C₂-C₁₂ alkynyl), two to nine carbon atoms (C₂-C₉ alkynyl), or two to six carbon atoms (C₂-C₆ alkynyl), or any value within these ranges, and having at least one carbon-carbon triple bond. Examples of alkynyl groups may be selected from the group consisting of ethynyl, propargyl, but-1-ynyl, but-2-ynyl and the like. The number of carbons referred to relates to the carbon backbone and carbon branching but does not include carbon atoms belonging to any substituents. Unless stated otherwise specifically in the specification, an alkynyl group is optionally substituted.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Aryl” refers to a ring system comprising at least one carbocyclic aromatic ring. In some embodiments, an aryl comprises from 6 to 18 carbon atoms. Aryl groups include groups having one or more 5-, 6-, 7- or 8-membered aromatic rings, including heterocyclic aromatic rings. The term heteroaryl specifically refers to aryl groups having at least one 5-, 6-, 7- or 8-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, P, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those that are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocyclic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein provided in a covalently bonded configuration in the compounds of the disclosure at any suitable point of attachment. In some embodiments, aryl groups contain one aromatic or heteroaromatic six-member ring and one or more additional five- or six-member aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents.

“Arylalkyl” groups are alkyl groups substituted with one or more aryl groups wherein the alkyl groups optionally carry additional substituents and the aryl groups are optionally substituted. Specific arylalkyl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms.

The terms “alkylene” and “alkylene group” are used synonymously and refer to a divalent group “—CH₂—” derived from an alkyl group as defined herein. The disclosure includes compounds having one or more alkylene groups. Alkylene groups in some compounds function as attaching and/or spacer groups. Compounds of the disclosure may have substituted and/or unsubstituted C₁-C₂₀ alkylene, C₁-C₁₀ alkylene and C₁-C₆ alkylene groups.

The terms “alkylenearyl” and “alkylenearyl group” are used synonymously and refer to an alkyl group substituted with an aryl group.

The terms “cycloalkylene” and “cycloalkylene group” are used synonymously and refer to a divalent group derived from a cycloalkyl group as defined herein. The disclosure includes compounds having one or more cycloalkylene groups. Cycloalkyl groups in some compounds function as attaching and/or spacer groups. Compounds of the disclosure may have substituted and/or unsubstituted C₃-C₃₀ cycloalkylene, C₃-C₁₈ cycloalkylene and C₃-C₆ cycloalkylene groups.

The terms “arylene” and “arylene group” are used synonymously and refer to a divalent group derived from an aryl group as defined herein. The disclosure includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as attaching and/or spacer groups. Arylene groups in some compounds function as chromophore, fluorophore, aromatic antenna, dye and/or imaging groups. Compounds of the disclosure include substituted and/or unsubstituted C₃-C₃₀ arylene, C₃-C₁₈ arylene and C₃-C₆ arylene groups.

The terms “heteroarylene” and “heteroarylene group” are used synonymously and refer to a divalent group derived from a heteroaryl group as defined herein. The disclosure includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as attaching and/or spacer groups. Heteroarylene groups in some compounds function as chromophore, aromatic antenna, fluorophore, dye and/or imaging groups. Compounds of the disclosure include substituted and/or unsubstituted C₃-C₃₀ heteroarylene, C₃-C₁₈ heteroarylene and C₃-C₆ heteroarylene groups.

The terms “alkenylene” and “alkenylene group” are used synonymously and refer to a divalent group derived from an alkenyl group as defined herein. The invention includes compounds having one or more alkenylene groups. Alkenylene groups in some compounds function as attaching and/or spacer groups. Compounds of the disclosure include substituted and/or unsubstituted C₂-C₂₀ alkenylene, C₂-C₁₀ alkenylene and C₂-C₅ alkenylene groups.

The terms “cycloalkenylene” and “cycloalkenylene group” are used synonymously and refer to a divalent group derived from a cycloalkenyl group as defined herein. The disclosure includes compounds having one or more cycloalkenylene groups. Cycloalkenylene groups in some compounds function as attaching and/or spacer groups. Compounds of the disclosure include substituted and/or unsubstituted C₃-C₃₀ cycloalkenylene, C₃-C₁₈ cycloalkenylene and C₃-C₆ cycloalkenylene groups.

The terms “alkynylene” and “alkynylene group” are used synonymously and refer to a divalent group derived from an alkynyl group as defined herein. The disclosure includes compounds having one or more alkynylene groups. Alkynylene groups in some compounds function as attaching and/or spacer groups. Compounds of the disclosure include substituted and/or unsubstituted C₂-C₂₀ alkynylene, C₂-C₁₀ alkynylene and C₂-C₅ alkynylene groups.

The terms “halo” and “halogen” can be used interchangeably and refer to a halogen group such as a fluoro (—F), chloro (—Cl), bromo (—Br) or iodo (—I)

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

The term “heteroalkyl”, as used herein, generally refers to an alkyl group as defined herein, wherein at least one carbon atom of the alkyl group is replaced with a heteroatom. In some instances, heteroalkyl groups may contain from 1 to 18 non-hydrogen atoms (carbon and heteroatoms) in the chain, or from 1 to 12 non-hydrogen atoms, or from 1 to 6 non-hydrogen atoms, or from 1 to 4 non-hydrogen atoms. Heteroalkyl groups may be straight or branched, and saturated or unsaturated. Unsaturated heteroalkyl groups have one or more double bonds and/or one or more triple bonds. Heteroalkyl groups may be unsubstituted or substituted. Exemplary heteroalkyl groups include, but are not limited to, alkoxyalkyl (e.g., methoxymethyl), and aminoalkyl (e.g., alkylaminoalkyl and dialkylaminoalkyl). Heteroalkyl groups may be optionally substituted with one or more substituents.

The term “carbonyl”, as used herein, for example in the context of C₁₋₆ carbonyl substituents, generally refers to a carbon chain of given length (e.g., C₁₋₆), wherein each of the carbon atom of a given carbon chain can form the carbonyl bond, as long as it is chemically feasible in terms of the valence state of that carbon atom. Thus, in some instance, the “C₁₋₆ carbonyl” substituent refers to a carbon chain of between 1 and 6 carbon atoms, and either the terminal carbon contains the carbonyl functionality, or an inner carbon contains the carbonyl functionality, in which case the substituent could be described as a ketone. The term “carboxy”, as used herein, for example in the context of C₁₋₆ carboxy substituents, generally refers to a carbon chain of given length (e.g., C₁₋₆), wherein a terminal carbon contains the carboxy functionality, unless otherwise defined herein.

As to any of the groups described herein that contain one or more substituents, it is understood that such groups do not contain any substitution or substitution patterns which are sterically impractical and/or synthetically non-feasible. In addition, the compounds of this disclosure include all stereochemical isomers arising from the substitution of these compounds.

Unless otherwise defined herein, optional substituents for any alkyl, alkenyl and aryl group includes substitution with one or more of the following substituents, among others:
halogen, including fluorine, chlorine, bromine or iodine;
pseudohalides, including —CN, —OCN (cyanate), —NCO (isocyanate), —SCN (thiocyanate) and —NCS (isothiocyanate);
—COOR, where R is a hydrogen or an alkyl group or an aryl group and more specifically where R is a methyl, ethyl, propyl, butyl, hexyl, or phenyl group all of which groups are optionally substituted;
—COR, where R is a hydrogen or an alkyl group or an aryl group and more specifically where R is a methyl, ethyl, propyl, butyl, hexyl, or phenyl group all of which groups are optionally substituted;
—CON(R)₂, where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂, where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂, where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring that can contain one or more double bonds and can contain one or more additional carbon atoms;
—SR, where R is hydrogen or an alkyl group or an aryl group and more specifically where R is hydrogen, methyl, ethyl, propyl, butyl, hexyl, decyl, or a phenyl group, which are optionally substituted;
—SO₂R, or —SOR, where R is an alkyl group or an aryl group and more specifically where R is a methyl, ethyl, propyl, butyl, hexyl, decyl, or phenyl group, all of which are optionally substituted;
—OCOOR, where R is an alkyl group or an aryl group;
—SO₂N(R)₂, where each R, independently of each other R, is a hydrogen, or an alkyl group, or an aryl group all of which are optionally substituted and wherein R and R can form a ring that can contain one or more double bonds and can contain one or more additional carbon atoms; and
—OR, where R is H, an alkyl group, an aryl group, or an acyl group all of which are optionally substituted. In a particular example R can be an acyl yielding —OCOR″, wherein R″ is a hydrogen or an alkyl group or an aryl group and more specifically where R″ is methyl, ethyl, propyl, butyl, hexyl, decyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups that contain one or more substituents, it is understood that such groups do not contain any substitution or substitution patterns which are sterically impractical and/or synthetically non-feasible. In addition, as further described herein, the compounds of this disclosure can include all stereochemical isomers (and racemic mixtures) arising from the substitution of these compounds.

Three-dimensional (3D) printing, also known as additive manufacturing, is a process used to create 3D objects of any shape from a digital design. 3D printing allows for the quick and efficient production of custom parts. In a typical 3D printing process, an initial material-layer is formed, followed by the sequential additional of subsequent material layers (or parts thereof), each building upon and connecting to the previous layer. This process continues until the entire designed 3D object is fully materialized.

Crystalline polymers possess excellent mechanical properties; however, their high crystallinity leads to undesirable shrinkage when used in 3D printing. This shrinkage makes crystalline polymers unsuitable for constructing 3D objects in extrusion-based additive manufacturing processes. The present application provides polymerizable compounds with reduced crystallinity by incorporating side chain liquid crystallinity units or crystallinity-disrupted comonomers that introduce defects into the polymer main chain. The curable compositions of the present disclosure comprising these polymerizable compounds are well suited for use in dental devices, such as spacers and aligners.

### Polymerizable Compounds

### A) Polymerizable Monofunctional Liquid Crystalline Compounds

In one aspect, polymerizable monofunctional liquid crystalline compounds are provided. These polymerizable monofunctional liquid crystalline compounds can be used as reactive diluents to reduce the viscosity of the curable compositions or as reactive groups for forming semicrystalline compounds having liquid crystalline domains pendant to the main polymer chain (i.e., polymer backbone).

In some embodiments, the polymerizable monofunctional liquid crystalline compound has the following structure (I):

G-A-X-L-Y-RM, (I)

wherein:
G is a polymerizable group;
A is an optional cycloalkylene or arylene linker;
X is an optional ester group;
L is an optional alkylene or heteroalkylene linker;
Y is an optional amide, ether, ester or urethane group; and
RM is a rigid moiety comprising one or more aromatic or cycloaliphatic rings.

In some embodiments, G is a polymerizable group capable of carrying out radical polymerization. Examples of polymerizable groups include, but are not limited to, an acrylate, methacrylate, vinyl acrylate, vinyl methacrylate, allyl ether, silene, alkyne, alkene, vinyl ether, maleimide, fumarate, maleate, itaconate, vinyl ester, vinyl ketone, epoxide, oxetane, cyclic esters, or styrenyl moiety, or a derivative thereof.

In some embodiments, G has one of the following structures:

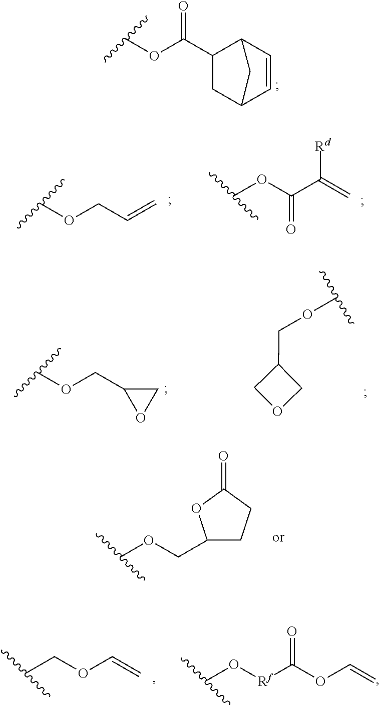

wherein:
R^{d} is H, halogen, or C₁-C₃ alkyl; and
R^{f} is C₁-C₆ alkylene.

In some embodiments, G has one of the following structures:

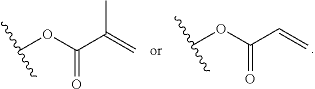

In some embodiments, A has one of the following structures:

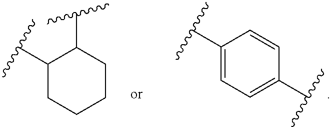 [SMILES: *C1CCCCC1* ; *c1ccc(*)cc1]

In some embodiments, L is C₁-C₁₂ alkylene.

In some embodiments, L is C₃-C₁₂ heteroalkylene.

In some embodiments, L has one of the following structures:

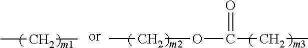

wherein m1, m2 and m3 are each independently an integer from 1 to 12.

In some embodiments, m1 is an integer from 2 to 8. In certain embodiments, m1 is 2, 4, 6, 8 or 10.

In some embodiments, m2 and m3 are each independently 2.

In some embodiments, RM comprises a cycloalkyl, aryl or heteroaryl alkylenearyl group or a combination thereof.

In some embodiments, RM is a divalent group having a C₅-C₁₈ cycloalkyl ring, a C₅-C₁₈ aryl ring, or a C₅-C₁₈ heteroaryl ring. The cycloalkyl, aryl, or heteroaryl ring may be a monocyclic structure or condensed ring structure. The monocyclic structure is preferably a 5- to 7-membered cyclic structure, or more preferably, a 5- or 6-membered cyclic structure. The heteroatom embedded in the hetero ring is preferably one or two selected from nitrogen, oxygen and sulfur atoms. “RM” may have two or more cyclic structures which may be same or different from each other, and in such a case, the cyclic structures may be linked by a single bond or a divalent group. Examples of the divalent group include, but are not limited to, an ethenyl, carbonyl, ester, acetylene, and azo group.

In some embodiments, RM has one of the following structures:

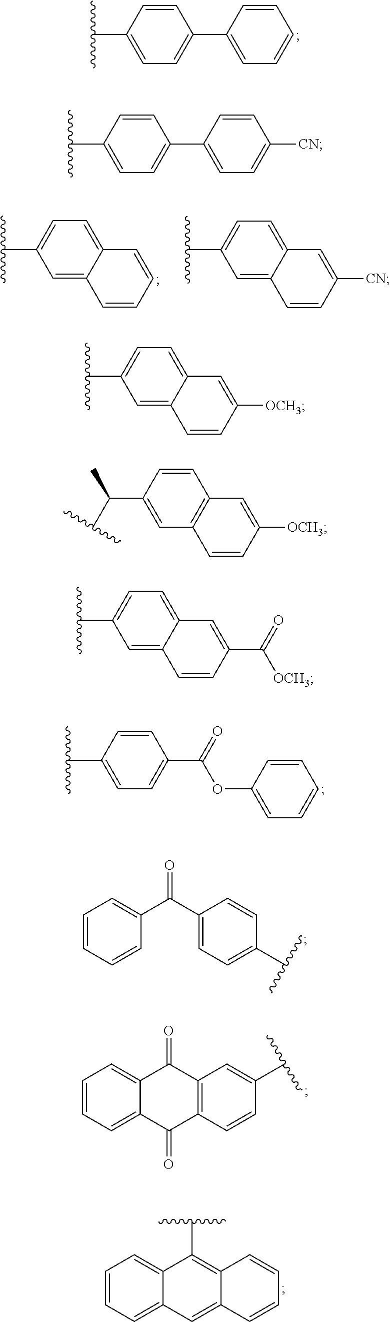

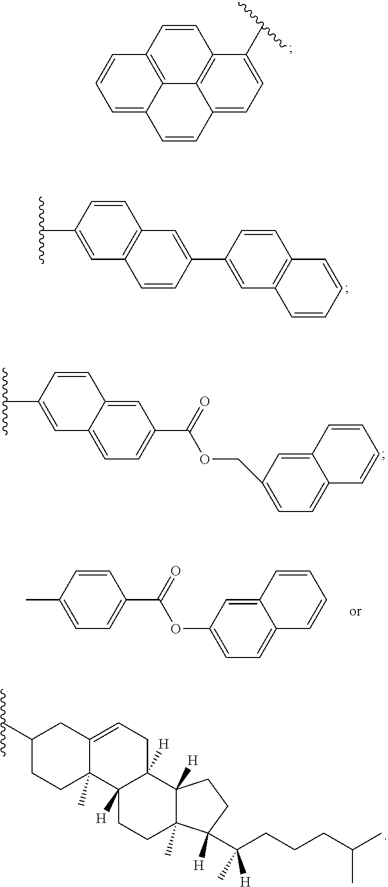 [SMILES: *c1ccc2ccc3cccc4ccc1c2c34 ; *c1ccc2cc(-c3ccc4ccccc4c3)ccc2c1 ; *c1ccc2cc(C(=O)OCc3ccc4ccccc4c3)ccc2c1 ; Cc1ccc(C(=O)Oc2ccc3ccccc3c2)cc1 ; *C1CC[C@@]2(C)C(=CC[C@H]3[C@@H]4CC[C@H]([C@@H](C)CCCC(C)C)[C@@]4(C)CC[C@@H]32)C1]

In some embodiments, the compound of structure (I) is a compound selected from Table 1.

TABLE 1
Exemplary Compounds of Structure (I)

| No. | Structure |
|---|---|
| I-1 | 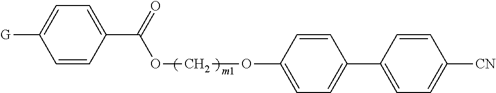 |
| | m1 is 2, 4, 6, or 8 |
| I-2 | 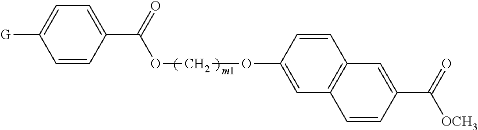 |
| | m1 is 2, 4, 6, or 8 |
| I-3 | 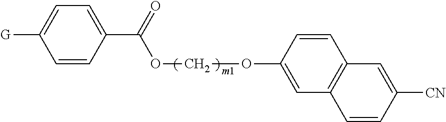 |
| | m1 is 2, 4, 6, or 8 |
| I-4 | 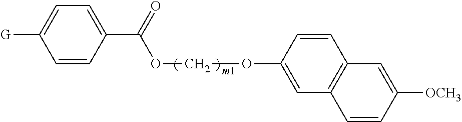 |
| | m1 is 2, 4, 6, or 8 |
| I-5 | 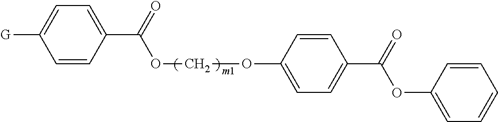 |
| | m1 is 2, 4, 6, or 8 |
| I-6 | 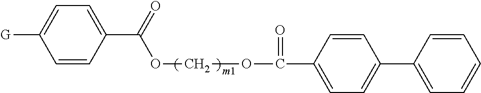 |
| | m1 is 2, 4, 6, or 8 |
| I-7 | 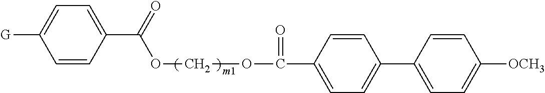 |
| | m1 is 2, 4, 6, or 8 |
| I-8 | 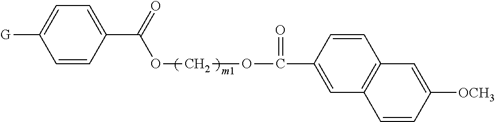 |
| | m1 is 2, 4, 6, or 8 |
| I-9 | 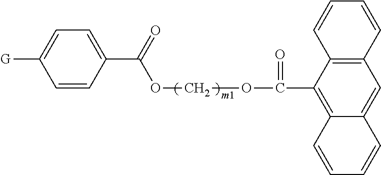 |
| | m1 is 2, 4, 6, or 8 |
| I-10 | 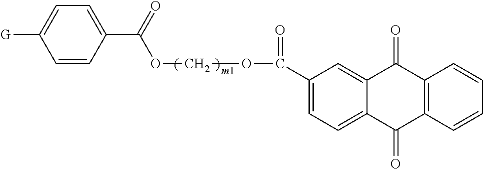 |
| | m1 is 2, 4, 6, or 8 |
| I-11 | 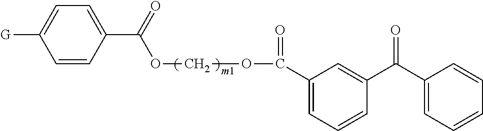 |
| | m1 is 2, 4, 6, or 8 |
| I-12 | |
| | 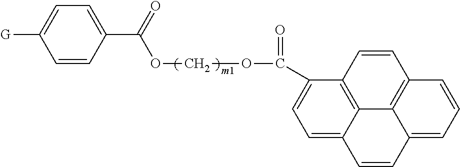 |
| | m1 is 2, 4, 6, or 8 |
| I-13 | 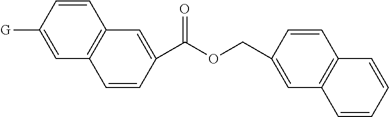 |
| I-14 | 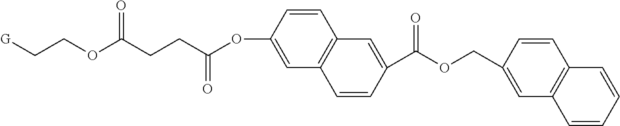 |
| I-15 | 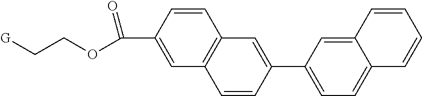 |
| I-16 | 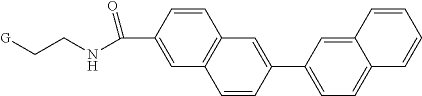 |
| I-17 | 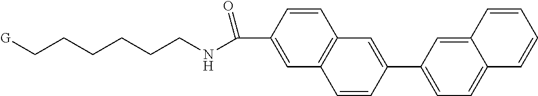 |
| I-18 | 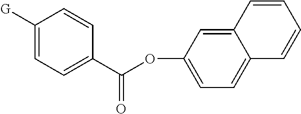 |
| I-19 | 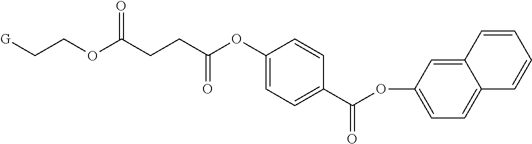 |
| I-20 | 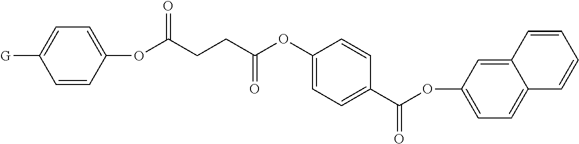 |
| I-21 | 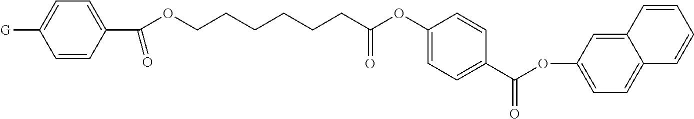 |
| I-22 | 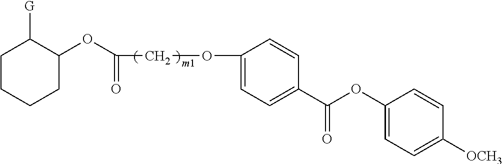 |
| | m1 is 4, 6, or 10 |
| I-23 | 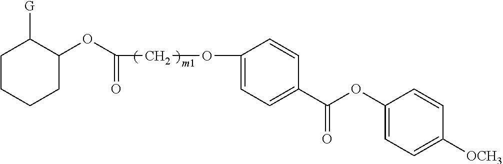 |
| | m1 is 4, 6, or 10 |
| I-24 | 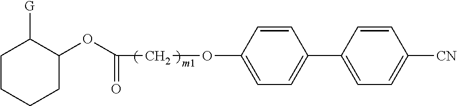 |
| | m1 is 4, 6, or 10 |
| I-25 | 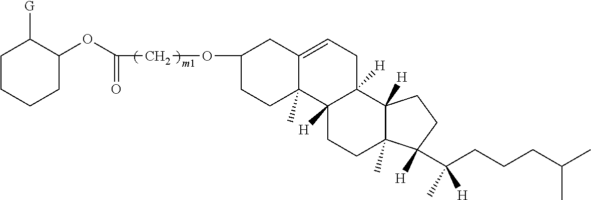 |
| | m1 is 4, 6, or 10 |
| I-26 | 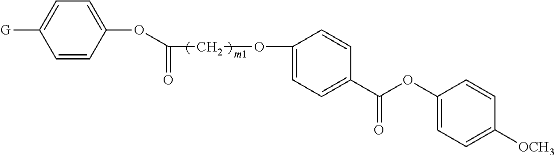 |
| | m1 is 4, 6, or 10 |
| I-27 | 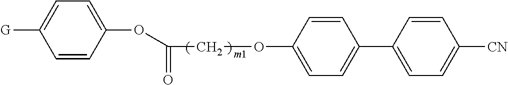 |
| | m1 is 4, 6 or 10 |
| I-28 | 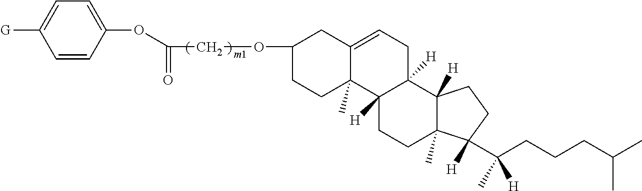 |
| | m1 is 4, 6, or 10 |
| I-29 | 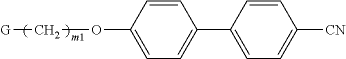 |
| | m1 is 4, 6 or 10 |
| I-30 | 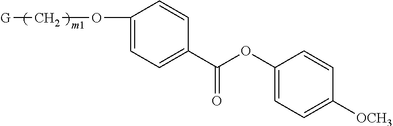 |
| | m1 is 4, 6 or 10 |
| I-31 | 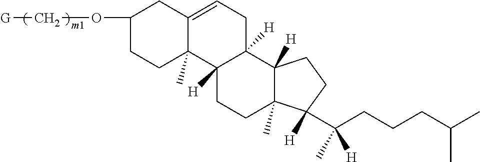 |
| | m1 is 4, 6 or 10 |
| I-32 | 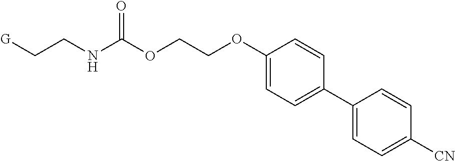 |
| I-33 | 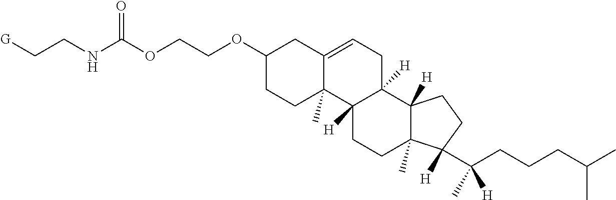 |
| I-34 | 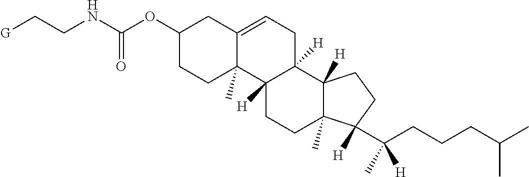 |
| I-35 | 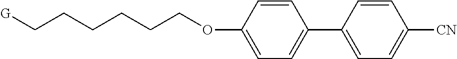 |

As used in Table 1 and throughout the application, G refers to a reactive functional group having one of the following structures:

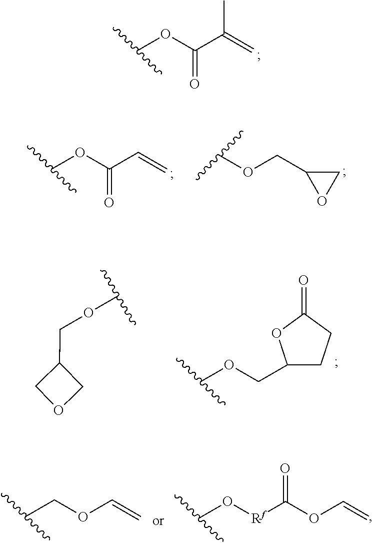

wherein R^{f} is C₁-C₆ alkylene.

It is understood that these reactive groups may connect to the liquid crystalline unit in other ways (esters, ethers, amides, amines, alkyl, etc.) and different spacer lengths or spacer types (ethers, esters, siloxanes, thioethers, etc.), and so the above represents a general guide for designing pendant liquid crystalline units of the present disclosure.

In some embodiments, the polymerizable monofunctional liquid crystalline compounds of structure (I) can be synthesized according to exemplary Scheme 1.

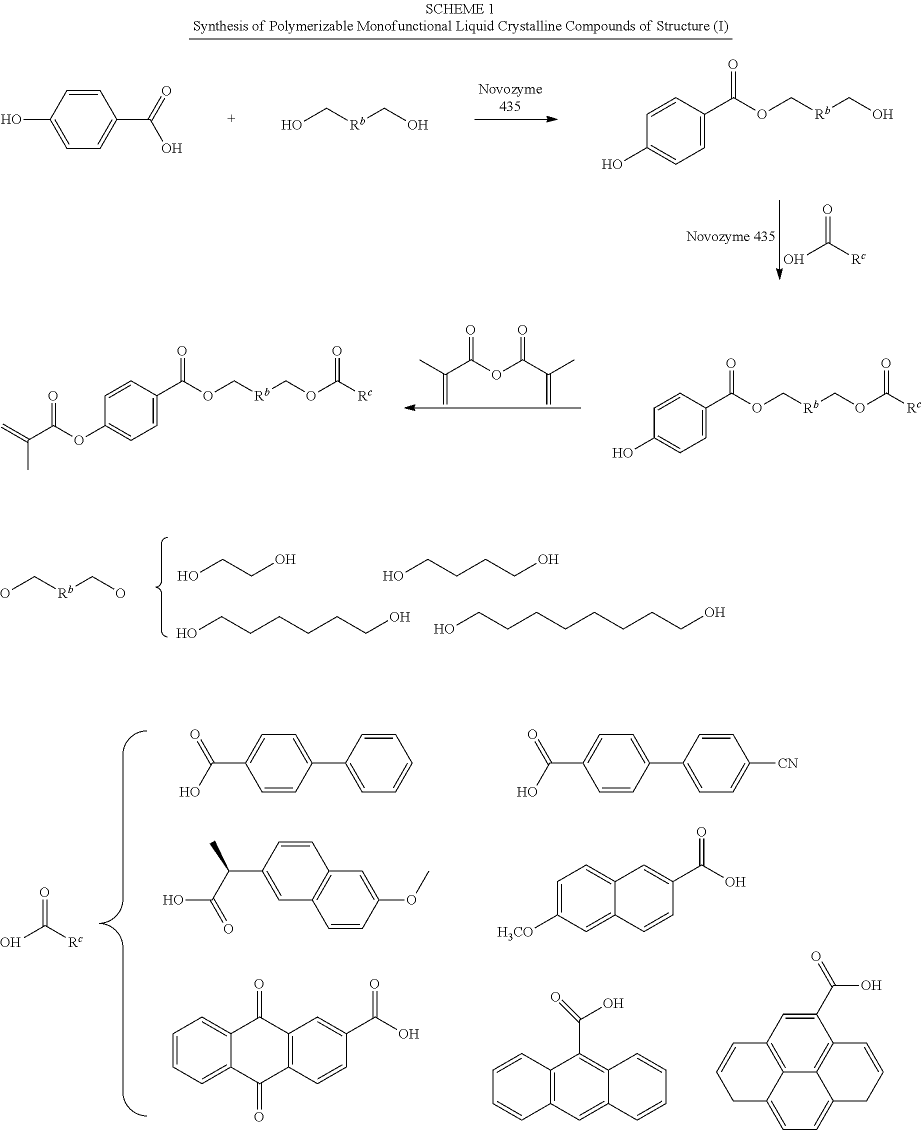

In some embodiments, more than one polymerizable liquid crystalline monomer can be used. Mixing liquid crystalline monomers can help to improve solubility and/or introduce additional features to the curable resins and resulting polymers, such as more than one melting point, more than one type of liquid crystal, and more than one liquid crystal transition temperature. Mixtures of polymerizable liquid crystalline monomers can also affect the crystallinity of the resulting polymers, influencing factors such as the size, shape, type, and number of crystalline domains, and even causing mixed or different liquid crystal formations within the resulting polymers. Variations among one or more additional polymerizable liquid crystalline monomers can be in any portion of the monomer, such as G, A, X, L, Y, or RM. Additionally, the mixture of these monomers may form a eutectic mixture, which helps to lower the melting temperature of the mixture, thereby facilitating the handling, mixing, processing, and printing of the mixture.

In some embodiments, the eutectic mixture comprises one or more additional monomers that contain a different polymerizable group G. A particularly useful feature of combining two or more polymerizable liquid crystalline monomers with different polymerizable groups that form a eutectic mixture is that during polymerization the reactivity ratio of the polymerizable groups can yield a perfectly alternating copolymer, random copolymer, block copolymer, and/or even two separate polymers. The resulting polymer(s) can have different melting points (Tm), crystal structures, crystalline phases, and solubility profiles compared to the monomer mixture. For instance, the resulting polymer may have one or more Tms that are 5° C., 10° C., 15° C., and/or even 20° C. higher than the monomer mixture, which can result in crystallization during polymerization at print or processing temperatures. One example is a eutectic mixture of a methacrylate polymerizable liquid crystalline monomer and a vinyl ester liquid crystalline monomer with similar but different mesogens. Upon polymerization, this mixture can produce two separate polymers, each with Tms corresponding to the two different mesogens, resulting in one or more melting points that are higher than the resin mixture. The use of eutectic mixtures to lower the Tm of a resin mixture is useful in the various different embodiments of this disclosure.

### B) Telechelic Polymers Comprising Liquid Crystalline Side Chains

Polymers with side chains or pendent liquid crystalline units are effective in controlling the overall crystallinity of the polymeric materials derived therefrom. In one aspect, the present disclosure provides telechelic polymers having liquid crystalline side chains. In various instances, provided herein are telechelic polymers (i.e., polymers consisting of a single monomer species A) and telechelic copolymers (i.e., polymers comprising 2, 3, 4, 5, or more different monomer species). In various cases, the telechelic copolymers described herein are telechelic block copolymers in which each monomer species is present in a “block” configuration within the copolymer structure. As further described herein, such block configuration can yield various polymer configurations, e.g., in cases where a telechelic block copolymer comprises 2 different monomer species A and B, block configurations such as AB, ABA, ABAB, AABB, etc. are possible.

Thus, throughout the present disclosure, the term “telechelic polymer” is further defined to include polymers consisting of (i) only one monomer species, and (ii) copolymers comprising 2, 3, 4, 5 or more different species of monomers. Such copolymers can be block copolymers described above or random copolymers. Furthermore, the term “telechelic,” as used herein in the context of polymers and block copolymers, generally refers to a polymer or oligomer capable of undergoing further polymerization through its reactive functional groups at its termini. As used herein, a telechelic polymer is generally characterized by a number-average molecular weight of at most about 50 kDa, 40 kDa, 30 kDa, 25 kDa, 20 kDa, 15 kDa, or 10 kDa. In some embodiments, the telechelics are of lower molecular weight such as 1 kDa to 10 kDa, or 2 kDa to 5 kDa, or 4 kDa to 8 kDa. In some embodiments, a mixture of different molecular weights of telechelics is used. Thus, in various instances, a telechelic polymer of this disclosure is capable of undergoing polymerization with itself, and/or one or more other telechelic polymers, telechelic block copolymers, telechelic oligomers, or monomers (e.g., reactive diluents) via the reactive functional groups at the termini. In some embodiments, a telechelic polymer comprises a reactive moiety enabling further polymerization reactions. Such polymerization reaction of telechelic polymer with other polymers, oligomers and/or monomers can occur during photo-curing, e.g., in instances where these components are part of a photo-curable resin as further described herein.

As further described herein, a telechelic polymer of the present disclosure comprising liquid crystalline side chains can enhance polymerization-induced phase separation (e.g., into one or more crystalline and/or amorphous phases) in a polymeric material into which the telechelic polymer is incorporated during, e.g., photo-curing. Hence, in some embodiments, a telechelic polymer herein can be used to control, at least in part, the number and/or sizes of the phase domains formed in a polymeric material upon photo-curing, and thereby provide materials with certain advantageous properties as described herein. Such phase control can be used to modify the transparency or clarity as well as the physical and mechanical properties of the resulting polymeric material. Furthermore, the chemical structure, monomer block configuration (e.g., AB, ABA, ABAB, etc.), and molecular weight of a telechelic polymer comprising 2 monomer species A and B can allow for controlling the morphology and properties of the resulting polymeric material into which the telechelic polymer is integrated.

A telechelic polymer of the present disclosure can comprise a range of degrees of terminal functionalization. In some cases, a population of telechelic polymers has complete or close to complete terminal substitution (e.g., by an acrylate or methacrylate moiety). For example, the population of telechelic polymers can have greater than 95%, greater than 97.5%, or greater than 99% terminal substitution. In some cases, the population of telechelic polymers has partial terminal substitution, for example between 60% and 95%, between 70% and 90%, between 80% and 95%, between 75% and 85%, or between 60% and 80%. In some cases, the terminal substitution of the population of telechelic polymers is greater than 95%, greater than 90%, greater than 80%, greater than 70%, greater than 60%, or greater than 50%. In some cases in which partial terminal substitution is optimal, the terminal substitution of the population of telechelic polymers is at most 90%, at most 85%, at most 80%, at most 70%, or at most 60%.

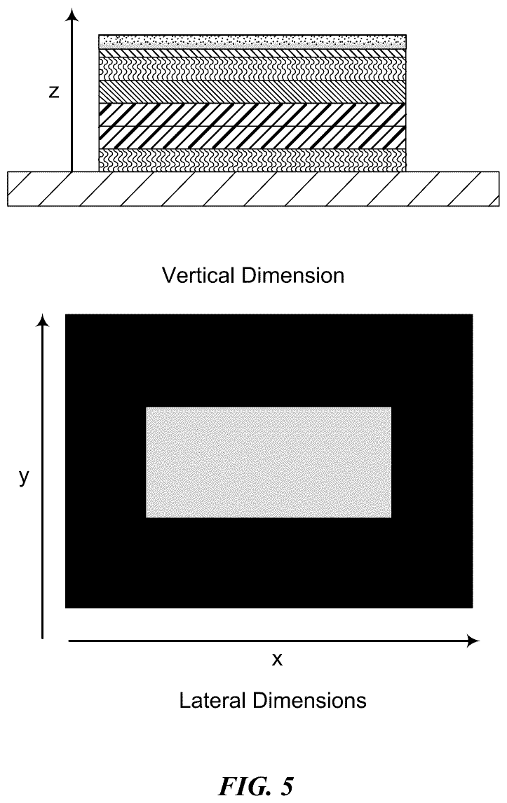

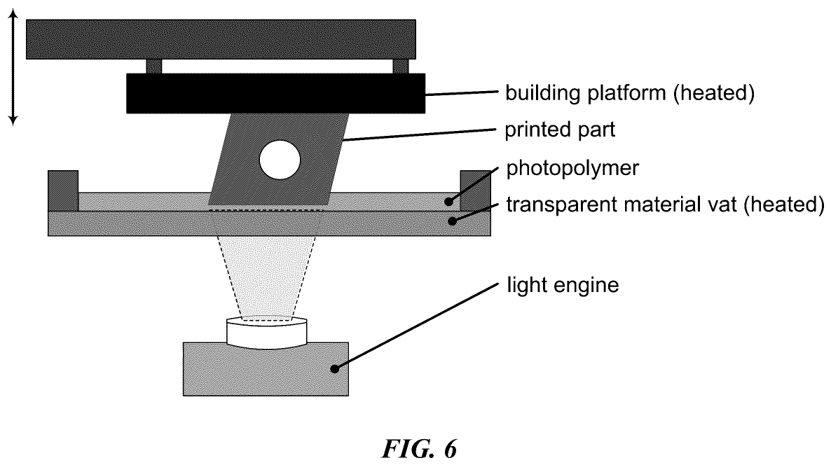

FIG. 7 illustrates a telechelic polymer 700, in accordance with some embodiments. Referring to FIG. 7, the telechelic polymer 700 includes a polymer backbone 710 comprising liquid crystalline side chains 720 attached thereto, and polymerizable groups 730 at termini of the polymer backbone 730. In some embodiments, the telechelic polymer 700 has the following structure (II):

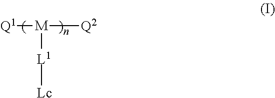

wherein:
M is an atom or group of atoms of the polymer backbone;
LC is a liquid crystalline moiety;
L¹ is an alkylene or heteroalkylene linker covalently bound the liquid crystalline moiety to the polymer backbone;
Q¹ and Q² are each independently an end-capping moiety comprising one or more reactive functional groups; and
n is an integer of one or greater.

The polymer backbone is an organic or an inorganic polymer backbone. In some embodiments, the polymer backbone includes repeating units derived from one or more monomers selected from acrylates, acrylic acids, siloxanes, hydroxystyrenes, methacrylates, vinyl esters, maleic esters, methacrylonitriles, and methacrylamides. In some embodiments, the polymer backbone comprises a polyhydroxystyrene, polyacrylate, polymethacrylate, substituted polyethylene, poly(vinyl ester), or polysiloxane backbone.

In some embodiments, substituted polyethylene is hydroxylated polyethylene.

In some embodiments, L¹ is a heteroalkylene linker.

In some embodiments, L¹ has one of the following structures:

—(CH₂)₃—S— or —CH₂—S—(CH₂)₃—.

In some embodiments, the LC moiety has the following structure:

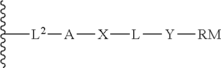

wherein:
L² is an alkylene linker;
A is an optional cycloalkylene or arylene linker;
X is an optional ester group;
L is an optional alkylene or heteroalkylene linker;
Y is an optional amide, ether, ester or urethane group; and
RM is a rigid moiety comprising one or more aromatic and/or cycloaliphatic rings.

In some embodiments, L² is C₁-C₁₂ alkylene. In some embodiments, L² is ethylene, propylene, butylene or hexylene. In some embodiments, L² is ethylene.

In some embodiments, A has one of the following structures:

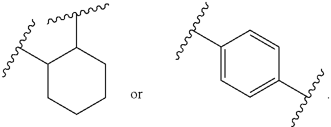 [SMILES: *C1CCCCC1* ; *c1ccc(*)cc1]

In some embodiments, L is C₁-C₁₂ alkylene.

In some embodiments, L is C₃-C₁₂ heteroalkylene. In some embodiments, L has one of the following structures:

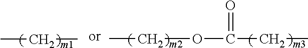

wherein m1, m2 and m3 are each independently an integer from 1 to 12.

In some embodiments, m1 is an integer from 2 to 8. In certain embodiments, m1 is 2, 4, 6, 8 or 10.

In some embodiments, m2 and m3 are each independently 2.

In some embodiments, RM comprises a cycloalkyl, aryl or heteroaryl alkylenearyl group or a combination thereof.

In some embodiments, RM is a divalent group having a C₅-C₁₈ cycloalkyl ring, a C₅-C₁₈ aryl ring, or a C₅-C₁₈ heteroaryl ring. The cycloalkyl, aryl, or heteroaryl ring may be a monocyclic structure or condensed ring structure. The monocyclic structure is preferably a 5- to 7-membered cyclic structure, or more preferably, a 5- or 6-membered cyclic structure. The heteroatom embedded in the hetero ring is preferably one or two selected from nitrogen, oxygen and sulfur atoms. “RM” may have two or more cyclic structures which may be the same or different from each other, and in such a case, the cyclic structures may be linked by a single bond or a divalent group. Examples of the divalent group include ethenyl, carbonyl, ester, acetylene, and azo group.

In some embodiments, RM has one of the following structures:

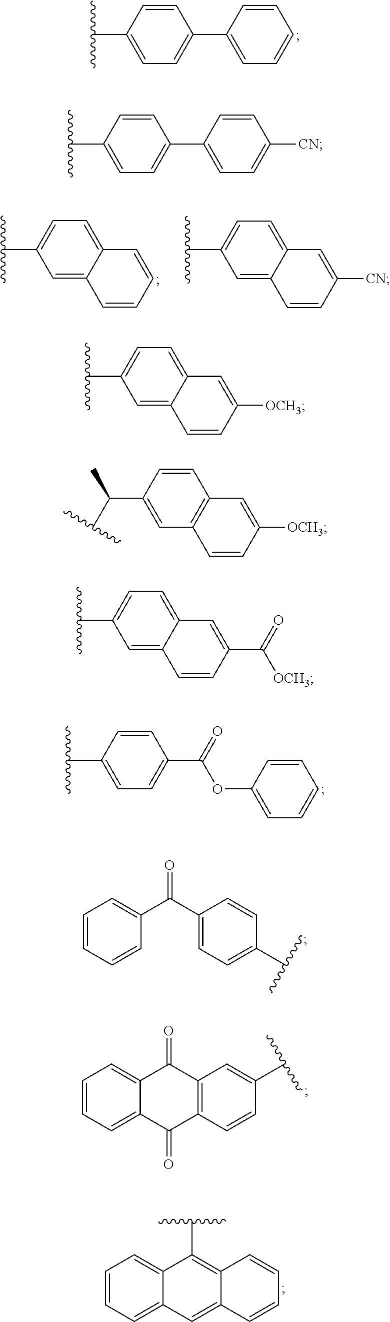

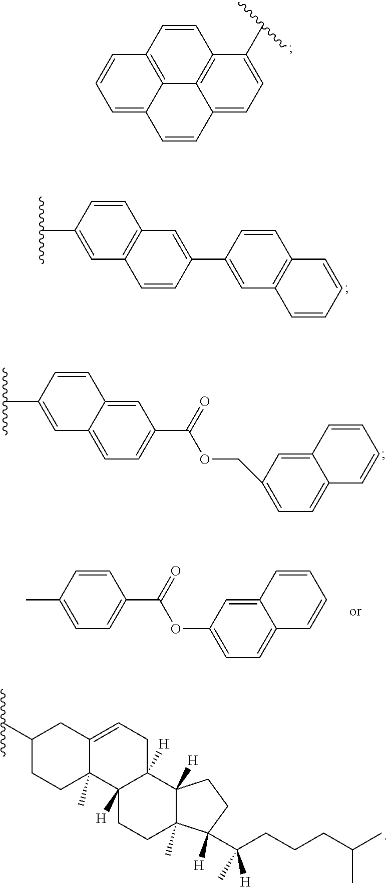 [SMILES: *c1ccc2ccc3cccc4ccc1c2c34 ; *c1ccc2cc(-c3ccc4ccccc4c3)ccc2c1 ; *c1ccc2cc(C(=O)OCc3ccc4ccccc4c3)ccc2c1 ; Cc1ccc(C(=O)Oc2ccc3ccccc3c2)cc1 ; *C1CC[C@@]2(C)C(=CC[C@H]3[C@@H]4CC[C@H]([C@@H](C)CCCC(C)C)[C@@]4(C)CC[C@@H]32)C1]

In various embodiments, a reactive functional group in each of Q¹ and Q² is capable of undergoing a polymerization reaction with a corresponding reactive functional group of another compound, e.g., another polymerizable compound or a polymerizable monomer, such as a reactive diluent. Thus, a reactive functional group herein is capable of undergoing an intermolecular polymerization reaction. The polymerization reaction can be any polymerization reaction known in the art, e.g., an addition polymerization or a condensation polymerization. In various cases, the polymerization reaction can be induced by electromagnetic radiation of appropriate wavelength, e.g., of the UV or visible region of the electromagnetic spectrum, and produce radicals or ions that can then initiate the polymerization reaction. In such cases, the polymerization can be a radically induced polymerization reaction, a cationically induced (e.g., epoxide cationic) polymerization reaction, or an anionically induced polymerization reaction. In some instances, a reactive functional group can be a Diels-Alder reactive group, or a group capable of undergoing a click reaction. In some embodiments, the reactive functional group is photoreactive. In some embodiments, mixtures of different reactive groups are used.

In various embodiments, a reactive functional group herein can comprise an alkene, alkyne, ketone, aldehyde, epoxide, nitrile, imine, amine, thiol, carboxylic acid, a derivative thereof, and/or any combination thereof. In some embodiments, a reactive functional group herein can comprise an acrylate, methacrylate, vinyl acrylate, vinyl methacrylate, allyl ether, silene, alkyne, alkene, vinyl ether, maleimide, fumarate, maleate, itaconate, vinyl ester, vinyl ketone, epoxide, oxetane, cyclic ester, or styrenyl moiety, or a derivative thereof.

In some embodiments, a reactive functional group herein comprises an alkene moiety, such as a vinyl group. In some instances, such reactive functional group can be selected from the group consisting of:

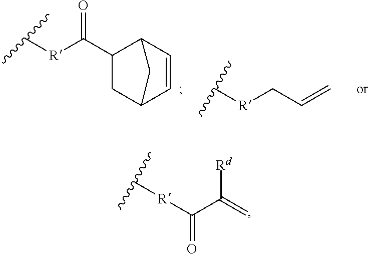

wherein:
R′ is an optional C₁-C₆ heteroalkylene linker; and
R^{d} is H, halogen, or C₁-C₃ alkyl.

In some embodiments, a reactive functional group herein comprises an epoxide moiety. In some embodiments, the reactive functional group comprises alkylene epoxide or cycloalkylene epoxide. In some cases, such reactive functional group can be:

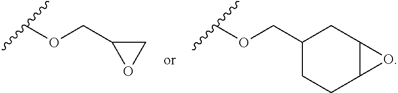 [SMILES: *OCC1CO1 ; *OCC1CCC2OC2C1]

In some embodiments, Q¹ or Q² has one of the following structures:

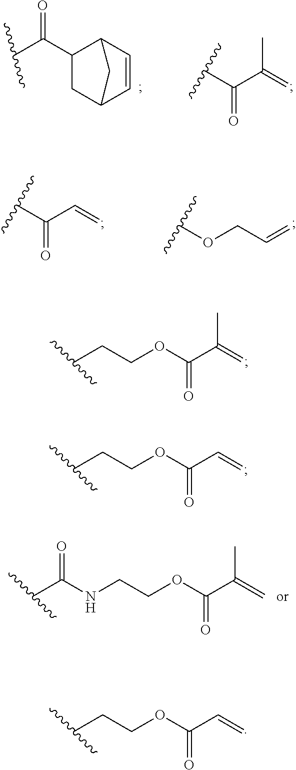

In some embodiments, n is an integer from 1 to 300. In some embodiments, n is an integer from 10 to 300, from 10 to 200, or from 10 to 150.

In some embodiments, the telechelic polymer of structure (II) has the following structure (IIA):

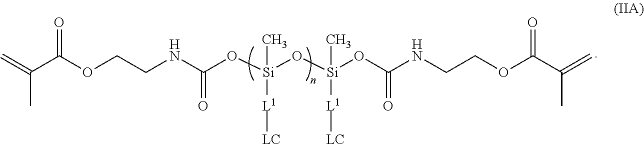

wherein L¹, LC and n are defined above.

In certain more specific embodiments, the telechelic polymer of structure (IIA) is a compound (II-1) having the following structure:

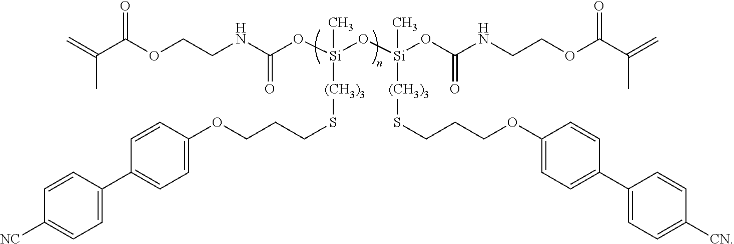

In some embodiments, the telechelic polymer of structure (II) has the following structure (IIB):

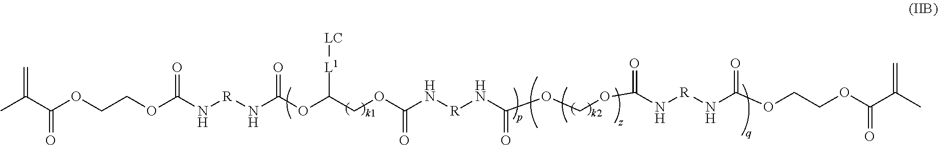

wherein:
R is a cycloalkylene, arylene, cycloalkylenedialkylene or arylenedialkylene group;
k1 and k2 are independently integer from 1 to 6;
p is an integer of one or greater; and
q is an integer of zero or greater.
   The L¹ and LC in the compound of structure (IIB) are defined above.

In some embodiments, R is cyclohexanedimethylene or cyclohexanemethylene.

In some embodiments, R has one of the following structures:

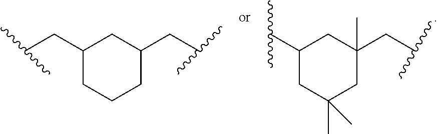 [SMILES: *CC1CCCC(C*)C1 ; *CC1(C)CC(*)CC(C)(C)C1]

In some embodiments, p is an integer from 1 to 300. In some embodiments, p is an integer from 1 to 150. In some embodiments, p is an integer from 1 to 100.

In some embodiments, q is 0. In some embodiments, q is an integer from 1 to 300. In some embodiments, p is an integer from 1 to 150. In some embodiments, p is an integer from 1 to 100.

In some more specific embodiments, the telechelic polymer of structure (IIB) is a compound II-2 having the following structure:

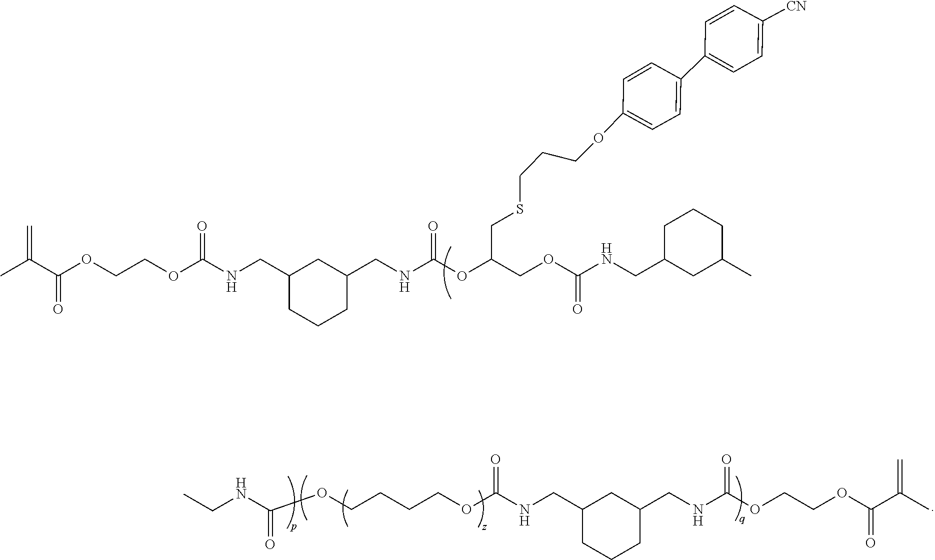

In some embodiments, q is 0. The telechelic polymer of structure (IIB) has the following structure (IIB-1):

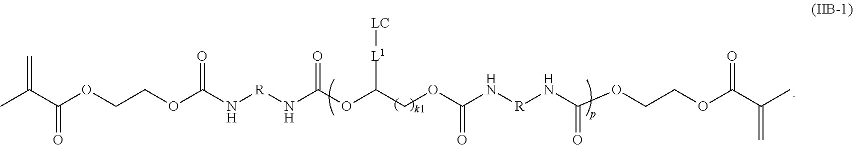

wherein R¹, L¹, LC, and p are defined above.

In certain more specific embodiments, the telechelic polymer of structure (IIB-1) is a compound (II-3) having the following structure:

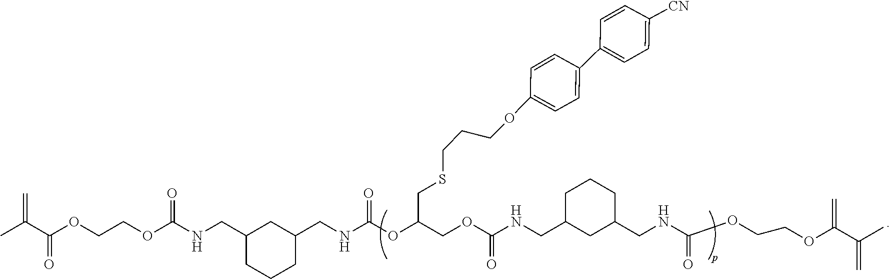

In some embodiments, the compounds of structure (II) can be prepared via a post-polymerization functionalization approach, where a liquid crystalline compound can be attached to a polymer having a complementary reactive group using any number of facile methods know in the art. Exemplary reactions include thiol-ene addition, alkene and azide cycloaddition, alkene and tetrazine inverse-demand Diels-Alder, alkene and tetrazole photoreaction or condensation reaction. For example, in some embodiments, the monofunctional liquid crystalline compound of structure (I) can be attached to a polymer backbone having a pendent thiol terminated group via thiol-ene addition to form the telechelic polymer of structure (II-2) or structure (II-3). In some embodiments, the compounds of structure (II) can be prepared by polymerization of a liquid crystalline-containing monomer with other comonomers. The liquid crystalline-containing monomer may contain thiols, epoxides, isocyanates, acrylates and enes polymerizable group. In some embodiments, the monofunctional liquid crystalline compound of structure (I) can be used as a monomer for forming the telechelic polymers of structure (II).

C) Polymerizable Compounds with Disrupted Crystallinity

Highly crystalline aliphatic-aromatic polyesters (also known as poly(alkylene terephthalates—PATs) such as polyethylene terephthalate (PET) are widely used for production of various light weight molded or extruded articles due to excellent mechanical properties. However, in some applications such as dental and medical applications, aliphatic-aromatic polyesters with reduced crystallinity are desirable due to their enhanced optical clarity, impact resistance and processability over the highly crystalline ones. The term “aliphatic-aromatic polyester”, as used herein, means a polyester comprising a mixture of residues from aliphatic dicarboxylic acids or diols and aromatic dicarboxylic acids or diols.

In embodiments of the present disclosure, the degree of crystallinity of the crystalline aliphatic-aromatic polyesters can be reduced by copolymerization of an aromatic diacid or diester with an additional crystallinity-disruptive diol comonomer that does not fit within a crystalline unit cell of the PAT. By “crystallinity-disruptive”, it is meant that the crystallinity of the polymer is disrupted so that the polymer is made less crystalline. The crystallinity-disruptive diol monomer introduces defects in the PAT crystalline structure. The defects will lower the melting temperature of PAT crystals, decrease the size of the crystals and crystalline domain, and thus make the polymeric material more processable. Suitable crystallinity-disruptive diol monomers are diol compounds including at least one of a branched aliphatic group, an aromatic group substituted with one or more alkyl groups, a sterically hindered group, and a stereoisomer. The crystallinity-disruptive diol monomers can be added at varying amounts, and effects of the crystallinity-disruptive diol monomers on the material properties can be measured using dynamic mechanical analysis (DMA) and DSC (Differential Scanning Calorimetry). Depending on the amount of the crystallinity-disruptive diol monomers added, the degree of crystallinity of the highly crystalline aromatic polyesters can be reduced by at least 5%, for example, from 5% to 90%, or more than 90%.

Accordingly, the present disclosure provides polymerizable aliphatic-aromatic polyester compounds having reduced crystallinity. The polymerizable crystallinity-disrupted compound can be oligomer or a polymer. In some embodiments, the polymerizable crystallinity-disrupted compound has a molecular weight from about 0.5 kDa to about 5 kDa and thus can be described as an oligomer. In other embodiments, the polymerizable crystallinity-disrupted compound has a number average molecular weight from about 5 kDa to about 50 kDa and thus can be described as a polymer. In still other embodiments, the molecular weight of the polymerizable crystallinity-disrupted compound is more than 50 kDa, such as up to 100 kDa, or up to 300 kDa. In some embodiments, the molecular weight distribution of the polymerizable crystallinity-disrupted compound is narrow, with a low PDI (polydispersity index) of 1 to 1.5, or a high PDI greater than 1.5 such as 1.5 to 2 or greater than 2.

In some embodiment, the polymerizable crystallinity-disrupted compound is a copolymer comprising at least two repeating units A and B, where unit A comprises a PAT and unit B comprises a PAT with disrupted crystallinity. The weight content of unit B, with reference to unit A, may be from 5 to 95 wt %, from 10 to 90 wt %, from 20 to 80 wt %, or from 30 to 55 wt %. In some embodiments, the molar ratio of unit A to unit B (A:B) is from (0.1 to 1):100, (1 to 10):100, (10 to 50):100, or (50 to 100):100. The copolymer may be a random copolymer or a block copolymer comprising two blocks A-B. In some embodiments, the copolymer has a viscosity of 100 cP to 1,000,000 cP (or greater, or even a solid) at temperatures used during a 3D printing process, a crystalline melt temperature of Tm in the range from 0° C. to 200° C., for example, from 70° C. to 150° C., and a glass transition temperature Tg in the range from −50° C. to 100° C.

In some embodiments, the polymerizable crystallinity-disrupted compound has the following structure (III):

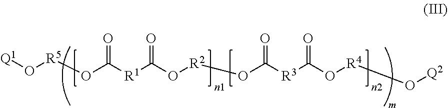

wherein:
R¹ and R³ are, at each occurrence, independently an arylene or heteroarylene group;
R² is, at each occurrence, independently a linear alkylene or arylene bis(alkylene ester) group;
R⁴ is, at each occurrence, independently a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
Q¹ and Q² are each independently an end-capping moiety comprising one or more reactive functional groups;
m is an integer of one or greater; and
n1 and n2 are, at each occurrence, independently an integer of one or greater for each integral value of m,
provided that if R¹ and R³ are the same, then R² and R⁴ are different, and if R² and R⁴ are the same, then R¹ and R³ are different.
R¹ and R³ may be substituted or unsubstituted. In some embodiments, R¹ and R³ are unsubstituted C₆-C₂₀ arylene or C₆-C₂₀ arylene substituted with one or more C₁-C₆ alkyl or C₁-C₆ alkoxy. In some more specific embodiments, R¹ and R³ are 1,4-phenylene, 1,2-phenylene, 1,3-phenylene, biphenylene, or naphthalene, wherein the 1,4-phenylene, 1,2-phenylene, 1,3-phenylene, biphenylene, or naphthalene is optionally substituted with one or more C₁-C₆ alkyl or C₁-C₆ alkoxy.

In some embodiments, R¹ and R³ may comprise a 5-membered heteroarylene structure such as 2,5-furanylene, 2,4-furanylene, 3,4-furanylene, 2,5-thienylene, 2,4-thienylene, or 3,4-thienylene, pyrrolyene, imidazolyene, pyrazolyene, oxazolyene, and thiazolyene. In some embodiments, R¹ and R³ may comprise of fused five membered and 6 membered heteroarylenes, such as benzofuranylene, isobezofuranylene, indolylene, isoindanolylene, benzothienylene, isobenzothienylene, benzeimidazolyene, indazolyene, benzoxazolyene, and benzothiazolyene. In some embodiments, the 2,5-furanylene, 2,4-furanylene, 3,4-furanylene, 2,5-thienylene, 2,4-thienylene, or 3,4-thienylene is optionally substituted with one or more C₁-C₆ alkyl or C₁-C₆ alkoxy.

In some embodiments, R¹ and R³ independently have one of the following structures:

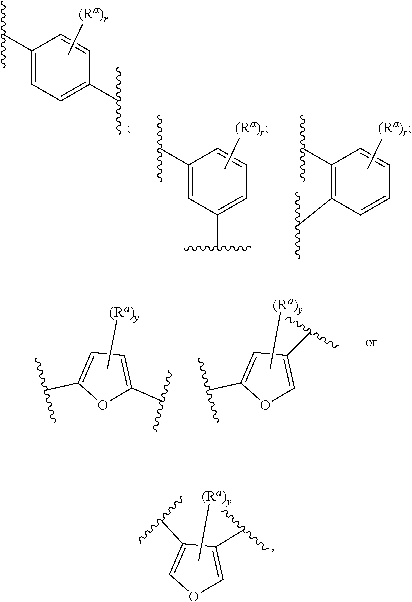

wherein:
R^{a} is, at each occurrence, C₁-C₆ alkyl or C₁-C₆ alkoxy;
r is an integer from 0 to 4; and
y is an integer from 0 to 2.

In some embodiments, R¹ and R³ have one of the following structures:

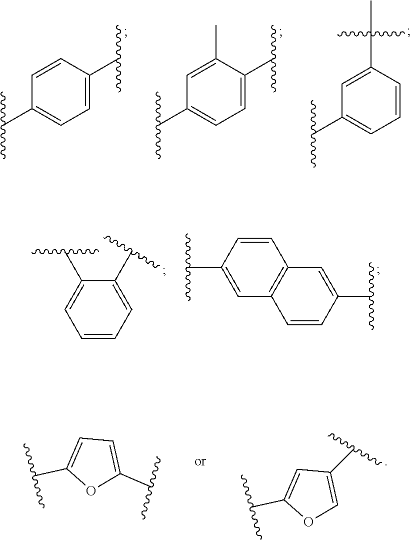

In some embodiments, R² is a linear C₁-C₁₂ alkylene. In some more specific embodiments, R² is ethylene, propylene, butylene, pentylene, hexylene, octylene, nonylene, decylene, or dodecylene.

In some embodiments, R⁴ is a linear or branched C₁-C₁₂ alkylene. In some more specific embodiments, R⁴ is ethylene, propylene, isopropylene, n-butylene, isobutylene, tert-butylene, pentylene, isopentylene, 3-methylpentylene, hexylene, octylene, nonylene, decylene, or dodecylene.

In some embodiments, R⁴ is a linear or branched C₂-C₁₂ heteroalkylene. In some more specific embodiments, R⁴ is alkylene oxide. In some embodiments, R⁴ is polyalkylene glycol (PAG). For example, in some embodiments, R⁴ is polyethylene glycol

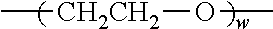

or polytetramethylene glycol

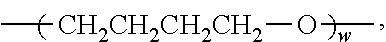

with w being an integer from 1 to 500. In some embodiments, w is an integer from 10 to 300, from 10 to 200, from 10 to 100 or from 1-40. The PAG has a number-average molecular weight ranging from 0.1 to 5 kDa, for example from 0.1 kDa to 4 kDa, from 0.1 kDa to 3 kDa, or from 0.1 kDa to 2 kDa.

In some more specific embodiments, R⁴ has one of the following structures:

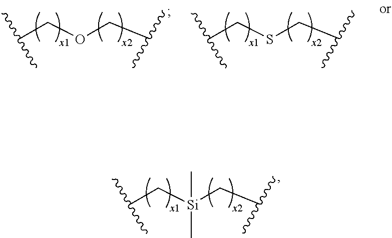

wherein x1 and x2 are independently an integer from 1 to 6.

In some embodiments, R⁴ is

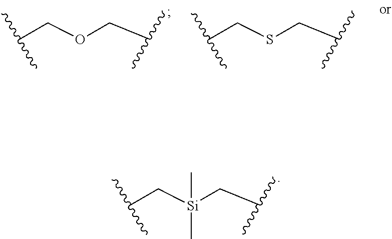

In some embodiments, R⁴ is arylene bis(alkylene ester). In some embodiments, R⁴ is phenylene bis(butylene ester), phenylene bis(pentalene ester), or phenylene bis(hexylene ester).

In some embodiments, R⁴ is bis(carboxyarylene) alkylene. In some embodiments, R⁴ is bis(carboxyphenylene) ethylene.

In some embodiments, R⁴ is bis(carboxycycloalkylene) alkylene. In some embodiments, R⁴ is bis(carboxycyclohexylene) ethylene.

In some embodiments, R⁵ is the same as R². In some embodiments, R⁵ is the same as R⁴. In some embodiments, R⁵ is C₁-C₁₂ alkylene. In some more specific embodiments, R⁵ is ethylene, propylene, butylene, pentylene, hexylene, octylene, nonylene, decylene, or dodecylene. In some embodiments, R⁵ is a branched C₂-C₁₂ alkylene. In some more specific embodiments, R⁵ is 3-methylpentylene.

In some embodiments, a reactive functional group in each of Q¹ and Q² is capable of undergoing a polymerization reaction (e.g. homopolymerization and/or copolymerization). In various embodiments, a reactive functional group in each of Q¹ and Q² is capable of undergoing a polymerization reaction with a corresponding reactive functional group of another compound, e.g., another polymerizable compound or a polymerizable monomer, such as a reactive diluent. Thus, a reactive functional group herein is capable of undergoing an intermolecular polymerization reaction. The polymerization reaction can be any polymerization reaction known in the art, e.g., an addition polymerization or a condensation polymerization. In various cases, the polymerization reaction can be induced by electromagnetic radiation of appropriate wavelength, e.g., of the UV or visible region of the electromagnetic spectrum, and produce radicals or ions that can then initiate the polymerization reaction. In such cases, the polymerization can be a radically induced polymerization reaction, a cationically induced (e.g., epoxide cationic) polymerization reaction, or an anionically induced polymerization reaction. In some instances, a reactive functional group can be a Diels-Alder reactive group, or a group capable of undergoing a click reaction.

In various embodiments, Q¹ and Q² can comprise an alkene, alkyne, ketone, aldehyde, epoxide, nitrile, imine, amine, carboxylic acid, thiol, alcohol, furan, diene (suitable in Diels-Alder), a derivative thereof, and/or any combination thereof. In some embodiments, Q¹ and Q² can comprise an acrylate, methacrylate, vinyl acrylate, vinyl methacrylate, allyl ether, silene, alkyne, alkene, vinyl ether, maleimide, fumarate, maleate, itaconate, vinyl ester, vinyl ketone, or styrenyl moiety, a derivative thereof, and/or any combination thereof.

In some embodiments, Q¹ and Q² comprise an alkene moiety, such as a vinyl group. In some instances, such reactive functional group can be selected from the group consisting of:

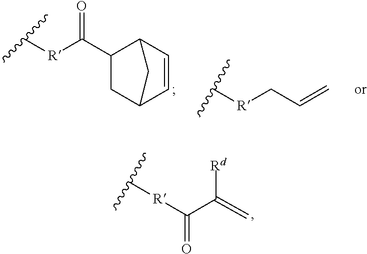

wherein:
R′ is an optional C₁-C₆ heteroalkylene linker; and
R^{d} is H, halogen, or C₁-C₃ alkyl.

In some embodiments, Q¹ and Q² herein comprise an epoxide moiety. In some embodiments, Q¹ and Q² comprise alkylene epoxide or cycloalkylene epoxide. In some cases, Q¹ and Q² can be:

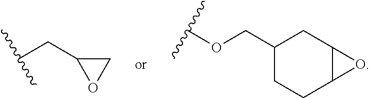 [SMILES: *CC1CO1 ; *OCC1CCC2OC2C1]

In some embodiments, Q¹ and Q² have one of the following structures:

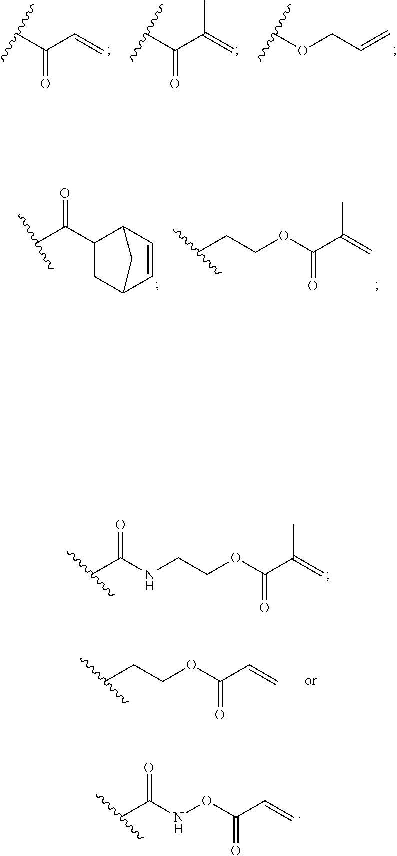

In some embodiments, Q¹ and Q² comprises a photoreactive functional group (e.g., maleimides).

In some embodiments, n1 and n2 are each independently an integer from 1 to 10, 10 to 20, 20 to 30, 30 to 40, 40 to 50, 50 to 100, 100 to 200, 200 to 300, 300 to 400, or 400 to 500. In some embodiments, n1 and n2 are each independently an integer from 10 to 500. In other embodiments, n1 and n2 are each independently an integer from 10 to 100, from 10 to 200, from 10 to 300, or from to 400.

In some embodiments, m is an integer from 1-50. In some embodiments, m is an integer from 1-20. In some embodiments, n is an integer from 1-10. In some embodiments, n is an integer from 1-5.

In some embodiments, R¹ is at each occurrence, independently an unsubstituted 1,4-phenylene group, R² is at each occurrence, independently linear alkylene, R³ is at each occurrence, independently a substituted or unsubstituted phenylene group or an isomer of an unsubstituted or substituted 1,4-phenylene group, and R⁴ is at each occurrence, independently a linear or branched alkylene or linear or branched heteroalkene group, and the polymerizable crystallinity-disrupted compound has the following structure (IIIA):

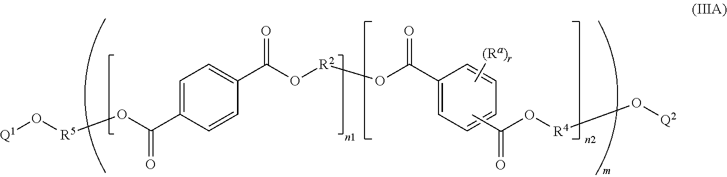

wherein:
R² is, at each occurrence, a linear C₁-C₁₂ alkylene group;
R⁴ is, at each occurrence, a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R^{a} is, at each occurrence, C₁-C₆ alkyl or C₁-C₆ alkoxy;
r is, at each occurrence, an integer from 0 to 4;
Q¹ and Q² are each independently an end-capping moiety comprising one or more reactive functional groups;
m is an integer of one or greater; and
n1 and n2 are, at each occurrence, independently an integer of one or greater for each integral value of m,
provided that if r is 0 and both phenylenes are unsubstituted 1,4-phenylene groups, then R² and R⁴ are different.

In some embodiments, R^{a} is C₁-C₆ alkyl at each occurrence. In some embodiments, R^{a} is methyl at each occurrence.

In some embodiments, r is 1 at each occurrence.

In some embodiments, (R^{a})ᵣ substitute phenylene is covalently bound to ester groups at para-positions, and the polymerizable crystallinity-disrupted compound of (IIIA) has the following structure (IIIB):

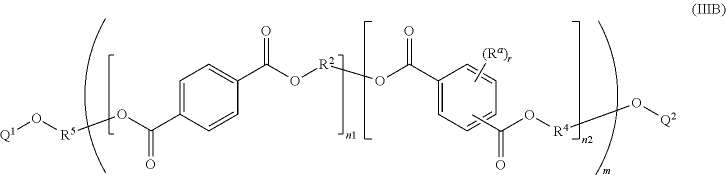

wherein:
R^{a} is, at each occurrence, C₁-C₆ alkyl or C₁-C₆ alkoxy;
r is, at each occurrence, an integer from 0 to 4.
R² is, at each occurrence, a linear C₁-C₁₂ alkylene group;
R⁴ is, at each occurrence, a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
Q¹ and Q² are each independently an end-capping moiety comprising one or more reactive functional groups;
m is an integer of one or greater; and
n1 and n2 are, at each occurrence, independently an integer of one or greater for each integral value of m,
provided that if r is 0, R² and R⁴ are different.

In some embodiments, (R^{a})ᵣ substitute phenylene is covalently bound to ester groups at ortho- or meta-positions, and the polymerizable crystallinity-disrupted compound of (IIIA) has the following structure (IIIC) or (IIID):

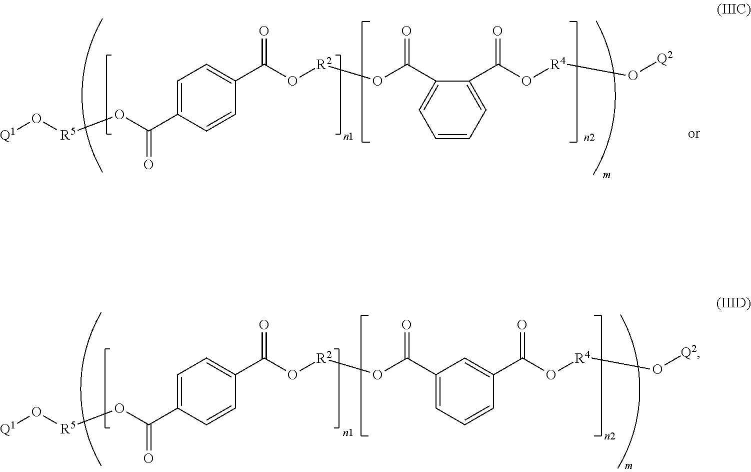

wherein:
R² is, at each occurrence, a linear C₁-C₁₂ alkylene group;
R⁴ is, at each occurrence, a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
Q¹ and Q² are each independently an end-capping moiety comprising one or more reactive functional groups;
m is an integer of one or greater; and
n1 and n2 are, at each occurrence, independently an integer of one or greater for each integral value of m.

In some embodiments, R⁴ is a phenylene bis(alkylene ester) or phenylene bis(heteroalkylene ester)group, and the polymerizable crystallinity-disrupted compound of structure (IIIA) has the following structure (IIIE):

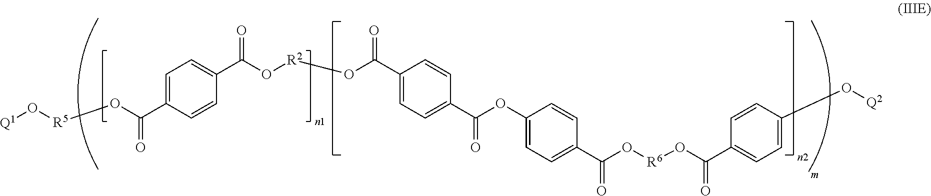

wherein:
R² is, at each occurrence, a linear C₁-C₁₂ alkylene group;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R⁶ is, at each occurrence, a linear or branched C₁-C₁₂ alkylene, C₃-C₁₈ cycloalkylene or linear or branched C₂-C₁₂ heteroalkylene group;
Q¹ and Q² are each independently an end-capping moiety comprising one or more reactive functional groups;
m is an integer of one or greater; and
n1 and n2 are, at each occurrence, independently an integer of one or greater for each integral value of m.

In some embodiments, R⁴ is a bis(carboxycyclohexylene) alkylene or bis(carboxycyclohexylene) heteroalkene group, and the polymerizable crystallinity-disrupted compound of structure (IIIA) has the following structure (IIIF):

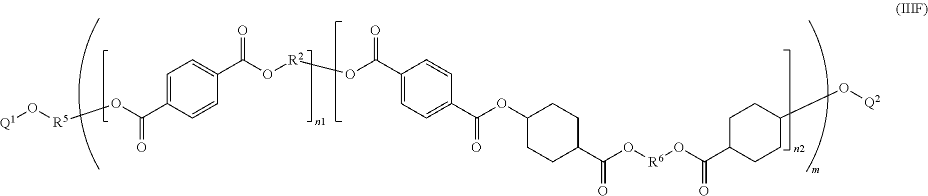

wherein:
R² is, at each occurrence, a linear C₁-C₁₂ alkylene group;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R⁶ is, at each occurrence, a linear or branched C₁-C₁₂ alkylene, C₃-C₁₈ cycloalkylene, or linear or branched C₂-C₁₂ heteroalkylene group;
Q¹ and Q² are each independently an end-capping moiety comprising one or more reactive functional groups;
m is an integer of one or greater; and
n1 and n2 are, at each occurrence, independently an integer of one or greater for each integral value of m.

In some embodiments, R⁶ has one of the following structures:

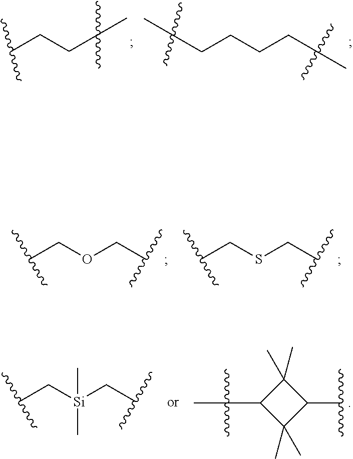

In some embodiments, two different crystallinity-disruptive monomers may be used, and the polymerizable crystallinity-disrupted compound of structure (IIIA) has the following structure:

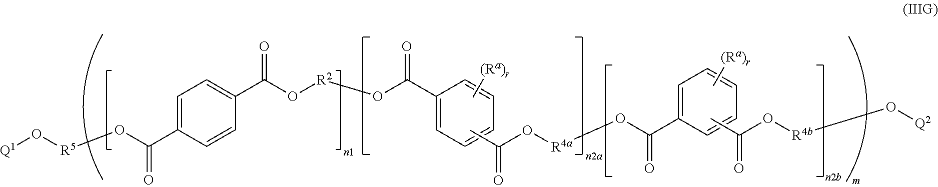

wherein:
R² is, at each occurrence, a linear C₁-C₁₂ alkylene group;
R^{4a} and R^{4b}, at each occurrence, a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group, provided that R^{4a} and R^{4b} are different;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R^{a} is, at each occurrence, C₁-C₆ alkyl or C₁-C₆ alkoxy;
r is, at each occurrence, an integer from 0 to 4;
Q¹ and Q² are each independently an end-capping moiety comprising one or more reactive functional groups;
m is an integer of one or greater; and
n1, n2a and n2b are, at each occurrence, independently an integer of one or greater for each integral value of m.
R^{4a} or R^{4b} is the same as R⁴. In some embodiments, R^{4a} or R^{4b} is a linear or branched C₁-C₁₂ alkylene. In some more specific embodiments, R^{4a} or R^{4b} is ethylene, propylene, isopropylene, n-butylene, isobutylene, tert-butylene, pentylene, isopentylene, 3-methylpentylene, hexylene, octylene, nonylene, decylene, or dodecylene.

In some embodiments, R^{4a} or R^{4b} is a linear or branched C₂-C₁₂ heteroalkylene. In some more specific embodiments, R^{4a} or R^{4b} is alkylene oxide. In some embodiments, R^{4a} or R^{4b} is polyalkylene glycol (PAG). For example, in some embodiments, R^{4a} or R^{4b} is polyethylene glycol

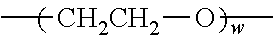

or polytetramethylene glycol

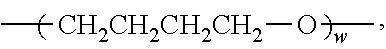

with w being an integer from 1 to 500. In some embodiments, w is an integer from 10 to 300, from 10 to 200, from 10 to 100 or from 1-40. The PAG has a number-average molecular weight ranging from 0.1 to 5 kDa, for example from 0.1 kDa to 4 kDa, from 0.1 kDa to 3 kDa, or from 0.1 kDa to 2 kDa.

In some more specific embodiments, R^{4a} or R^{4b} has one of the following structures:

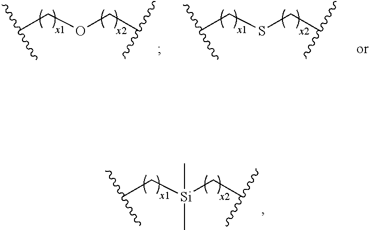

wherein x1 and x2 are independently an integer from 1 to 12.

In some embodiments, R^{4a} or R^{4b} is

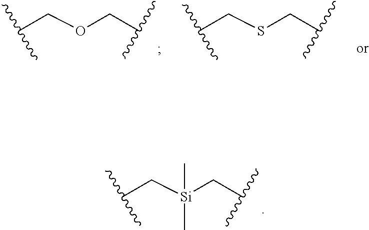

In some embodiments, R^{4a} or R^{4b} is arylene bis(alkylene ester). In some embodiments, R⁴ is phenylene bis(butylene ester), phenylene bis(pentalene ester), or phenylene bis(hexylene ester).

In some embodiments, R^{4a} or R^{4b} is bis(carboxyarylene) alkylene. In some embodiments, R⁴ is bis(carboxyphenylene) ethylene.

In some embodiments, R^{4a} or R^{4b} is bis(carboxycycloalkylene) alkylene. In some embodiments, R⁴ is bis(carboxycyclohexylene) ethylene.

In some embodiments, n1, n2a, and n2b are each independently an integer from 1 to 5, 5 to 10, 10 to 20, 20 to 30, 30 to 40, 40 to 50, 50 to 100, 100 to 200, 200 to 300, 300 to 400, or 400 to 500. In some embodiments, n1, n2a, and n2b are each independently an integer from 5 to 500. In other embodiments, n1, n2a, and n2b are each independently an integer from 5 to 100, from 10 to 200, from 10 to 300, or from 10 to 400.

In some embodiments, the polymerizable crystallinity-disrupted compound of structure (III) is a compound selected from Table 2.

TABLE 2
Exemplary Polymerizable Crystallinity-Disrupted Compounds of Structure (III)

| # | Structure |
|---|---|
| III-1 | 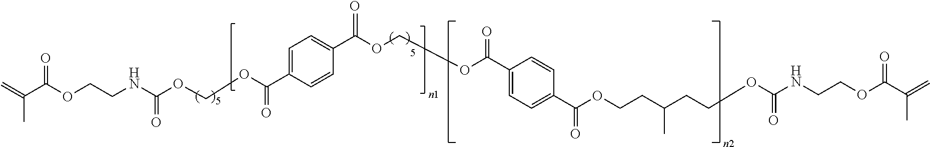 |
| III-2 | 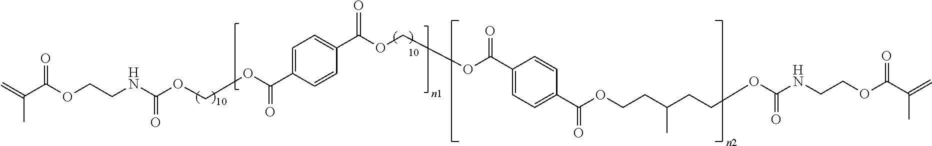 |
| III-3 | 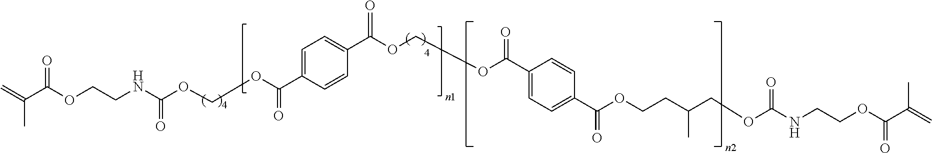 |
| III-4 | 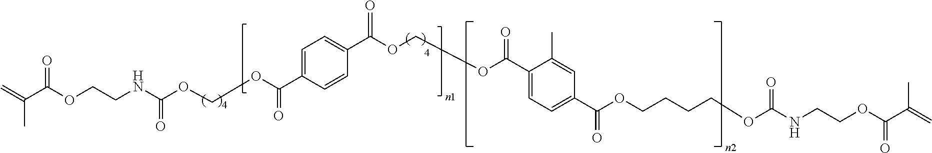 |
| III-5 | 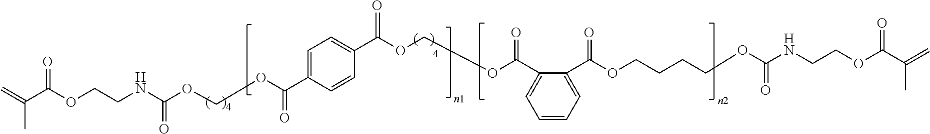 |
| III-6 | 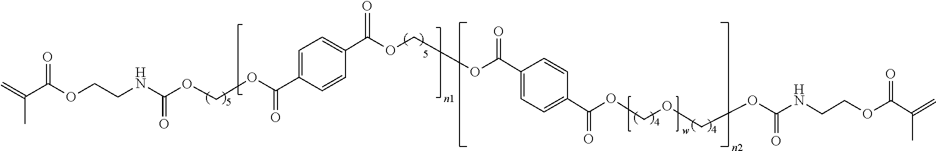 |
| | w = 10-40 |
| III-7 | 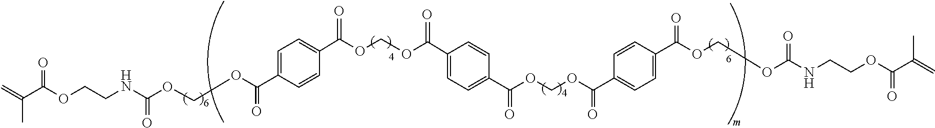 |
| III-8 | 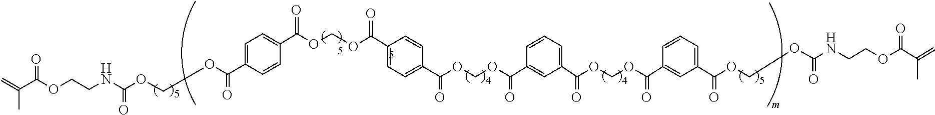 |
| III-9 | 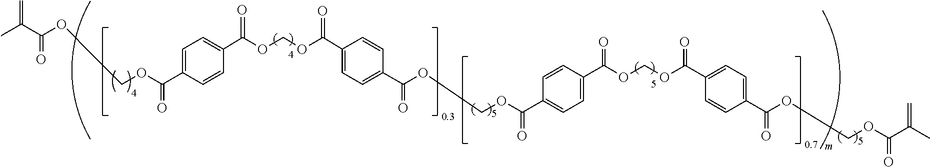 |
| III-10 | 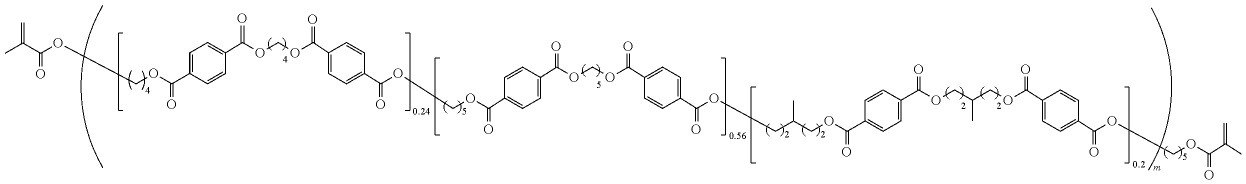 |
| III-11 | 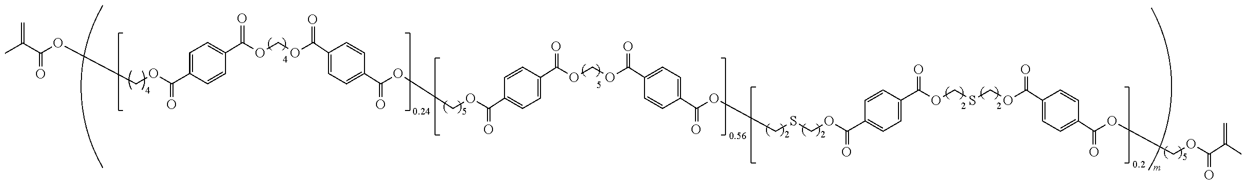 |
| III-12 | 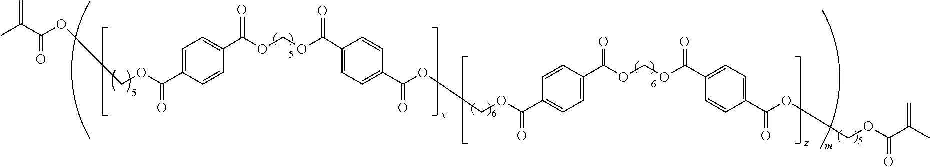 |
| | x:z = 0.7:0.3; |
| | x:z = 0.6:0.4; |
| | x:z = 0.5:0.5; |
| | x:z = 0.6:0.6; |
| | x:z = 0.3:0.7 |

Aliphatic dicarboxylic acids or diols and aromatic dicarboxylic acids or diols can be used for preparation of polymerizable crystallinity-disrupted compounds of structure (III). In some embodiments, the preparation of these polymerizable crystallinity-disrupted compounds of structure (III) can be carried out by polycondensation of an unsubstituted aromatic dicarboxylic acid with two or more diols, at least one of which is a crystallinity-disruptive diol.

As used herein, the term “dicarboxylic acid” is intended to include the acid itself and residues thereof as well as any derivative of the acid, including its associated acid halides, esters, half-esters, salts, half-salts, anhydrides, mixed anhydrides, or mixtures thereof or residues thereof useful in a reaction process with a diol to make polyester.

Examples of unsubstituted aromatic dicarboxylic acids include, but are not limited to, 1,4-terephthalic acid, biphenyl dicarboxylic acid, 2,6-naphthalene dicarboxylic acid, and thiophenedicarboxylic acid.

Examples of substituted aromatic diesters include, but are not limited to, di-tert-butyl terephthalate and diethyl terephthalate.

Examples of linear aliphatic dicarboxylic acids, include, but are not limited to, malonic acid (HO₂CCH₂COOH), succinic acid (HO₂C(CH₂)₂COOH), glutaric acid (HO₂C(CH₂)₃COOH), adipic acid (HO₂C(CH₂)₄COOH), pimelic acid (HO₂C(CH₂)₅COOH), suberic acid (HO₂C(CH₂)₆COOH), azelaic acid (HO₂C(CH₂)₇COOH), and sebacic acid (HO₂C(CH₂)₈COOH).

Examples of unsubstituted aromatic diols include, but are not limited to, 1,4-hydroquinone, 4,4′-biphenol, and 2,6-naphthalenediol.

Examples of linear aliphatic diols include, but are not limited to ethylene glycol, propylene glycol, 1,4-butanediol, 1,5-pentanediol, 1,6-hexanediol, 1,8-octanediol, and 1,10-decanediol.

Examples of crystallinity-disruptive dicarboxylic acid monomer include, but are not limited to, 1,2-terephthalic acid, isophthalic acid, 2-methylterephthalic acid, 2-(tert-butyl)terephthalic acid, 1,4-Cyclohexanedicarboxylic acid, 2,4-furandicarboxylic acid, and 2,5-furandicarboxylic acid.

Examples of crystallinity-disruptive diol monomers include, but are not limited to, 2-methyl-butanediol. 2,2,4-trimethyl-1,3-pentanediol, 2-methyl-1,3-pentanediol, 2-ethyl-1,3-hexanediol, 2-methyl-1,3-propanediol, 2,2-dimethyl-1,3-propanediol, dibutyl 1,3-propanediol, 3-methyl pentanediol, polyalkylene glycols such as poly(ethylene glycol), and poly(tetrahydrofuran), 1,4-cyclohexanedimethanol, 2,2,4,4-tetramethyl-1,3-cyclobutanediol, 2,2′-thiodiethanol, and 2-methylhydroquinone.

Examples of arylene bis(alkylene ester) monomers include, but are not limited to, bis(4-hydroxy butyl) terephthalate, bis(5-hydroxy pentyl) terephthalate, and bis(6-hydroxy hexyl terephthalate.

Examples of arylene bis(heteroalkylene ester) monomers include, but are not limited to, di[2-(2-hydroxyethylthio)ethyl]terephthalate.

In some embodiments, the diol monomer that can be used as a crystallinity-disruptive unit for preparing compounds of structure (IIIE) or (IIIF) has the following structure (IV):

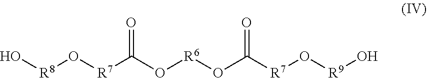

wherein:
R⁶ is a linear or branched alkylene, a substituted or unsubstituted cycloalkylene or linear or branched heteroalkylene group;
R⁷ is, at each occurrence, independently a substituted or unsubstituted arylene or substituted or unsubstituted cycloalkylene group; and
R⁸ and R⁹ are independently a linear or branched C₁-C₁₂ alkylene group. In some embodiments, the crystallinity-disruptive diol monomer of structure (IV) has the following structure (IVA) or (IVB):

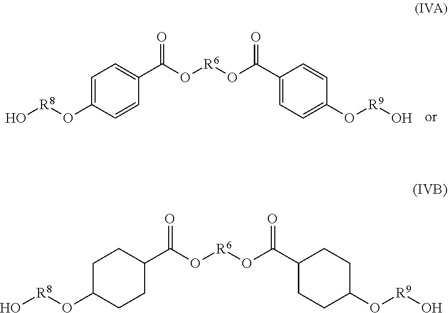

wherein:
R⁶ is a linear or branched C₁-C₁₂ alkylene, substituted or unsubstituted C₃-C₁₈ cycloalkylene, or linear or branched C₂-C₁₂ heteroalkylene group; and
R⁸ and R⁹ are independently a linear or branched C₁-C₁₂ alkylene group.

The crystallinity-disruptive diol monomer of structure (IV) can exist as a mixture of crystalline units and crystallinity-disruptive units because the arylene or cycloalkylene ring, such as phenylene or cyclohexylene ring can provide chair, boat, and twist conformations as well as different isomers depending on the substitution. The presence of different isomers limits the degree of crystallinity in the compounds of structure (III). By controlling the conformation of the ring structure, the degree of crystallinity in the compounds of structure (III) can be regulated.

In some embodiments, a stoichiometric excess of diol is used relative to the dicarboxylic acid to yield a hydroxy terminated polyester, which can be further reacted with a polymerizable compound comprising at least one reactive functional group to afford the polymerizable crystallinity-disrupted ester compound of the present disclosure. In some embodiments, the hydroxy terminal groups are reacted with a polymerizable end-capping compound, for example, methacrylic acid, acryloyl chloride, allyl chloroformate, 2-mercaptopropionic acid, or 2-isocyanatoethyl methacrylate to yield the compounds of structure (III).

In some embodiments, a stoichiometric excess of dicarboxylic acid is used relative to the diol to yield a carboxylic acid (or ester) terminated polyester, which can be further reacted with a polymerizable compound, comprising at least one reactive functional group to afford the polymerizable crystallinity-disrupted ester compound of the present disclosure. In some embodiments, the carboxyl terminal groups are reacted with a polymerizable end-capping compound, for example, 2-hydroxyethylmethacrylate, 5-norbornene-2-methanol, epichlorohydrin, or allyl alcohol to yield the polymerizable crystallinity-disrupted compounds of structure (III).

In some embodiments, polymerizable crystallinity-disrupted compounds of the present disclosure are mixed with oligomers and/or polymers of similar structure to that found in the repeating unit n1 or n2. For example, polybutylene terephthalate homopolymer of 50 kDa can be mixed with a polymerizable crystallinity-disrupted compound of structure (III) with n1 repeat units of butylene terephthalate and n2 of a moiety such as decane terephthalate that disrupts the crystallinity. The amount of polymerizable crystallinity-disrupted compound of structure (III) that can be mixed into a crystalline polymer is determined by the degree of crystallinity desired but can range from 5 to 95 wt %, 25 to 75 wt %, 30 to 70 wt %, 40 to 60 wt %, or 50 to 95 wt %.

The ratio (n1:n2) of crystalline unit n1 to crystallinity-disruptive unit n2 in polymerizable crystallinity-disrupted compounds of structure III are adjusted to meet the desired property needs, and may range from 1:1 to 5:1, 1:1 to 10:1, 1:1 to 100:1, 5:1 to 30:1, 10:1 to 50:1, or from 0.5:1 to 1:1. In some embodiments, when polymerizable crystallinity-disrupted compounds of structure (III) are mixed with homopolymers and/or oligomers having the repeating units of n1, the ratio (n1:n2) can range from 0:1 to 10:1, 0:1 to 2:1, or 0:1 to 1:1.

### Curable Composition

In some embodiments, curable compositions provided herein possess low viscosity, which allows for ease of dispensing and application during the additive manufacturing process. Accordingly, different additive manufacturing techniques such as materials jetting, vat photopolymerization, binder jetting, etc., can be used.

In some embodiments, the present disclosure provides curable compositions (i.e., curable resins) comprising one or more polymerizable compounds of structure (I), (II) or (III). A curable composition herein can be a photo-curable composition, a thermo-curable composition, or a combination thereof.

In some cases, the curable composition comprises 10 to 70 wt %, 10 to 60 wt %, 10 to 50 wt %, 10 to 40 wt %, 10 to 30 wt %, 10 to 25 wt %, 20 to 60 wt %, 20 to 50 wt %, 20 to 40 wt %, 20 to 35 wt %, 20 to 30 wt %, 25 to 60 wt %, 25 to 50 wt %, 25 to 45 wt %, 25 to 40 wt %, or 25 to 35 wt %, based on the total weight of the composition, of a polymerizable compound of structure (I), (II) or (III). In certain embodiments, the curable composition may comprise 25 to 35 wt %, based on the total weight of the composition, of a polymerizable compound of structure (I), (II) or (III). In certain embodiments, the curable composition may comprise 20 to 40 wt %, based on the total weight of the composition, of a polymerizable compound of structure (I), (II) or (III).

In other embodiments, the polymerizable compounds of structure (I), (II) or (III) are used at higher weight percents, such as greater than 70%, greater than 80%, greater than 90%, or greater than 95%, or are used without other comonomers. In some embodiments, mixtures of different monomers of structure (I) are used without other comonomers. In some embodiments, telechelic compounds of structure (II) are used without other comonomers. In some embodiments, polymerizable crystallinity-disrupted compounds of structure (III) are used without other comonomers. In some embodiments, mixtures of structures (I), (II), and/or (III) are used without comonomers that do not fit structures (I), (II), or (III).

In various cases, the terminal reactive functional groups of polymerizable compounds of structure (I), (II) or (III) enable photo-polymerization reactions. Such photo-polymerization reaction of polymerizable compounds of structure (I), (II) or (III) can occur during photo-curing.

In some embodiments, the curable composition further comprises an initiator. In some embodiments, the initiator is a photoinitiator. In some embodiments, photoinitiators may be useful for various purposes, including for curing polymers, including those that can be activated with light and initiate polymerization of the polymerizable components of the formulation. In some embodiments, the photoinitiator is a radical photoinitiator and/or a photoacid initiator. In some embodiments, the initiator comprises a photo base generator.

In some embodiments, the photoinitiator is a free radical photoinitiator. In certain embodiments, the free radical photoinitiator comprises an alpha hydroxy ketone moiety (e.g., 2-hydroxy-2-methylpropiophenone or 1-hydroxycyclohexyl phenyl ketone), an alpha-amino ketone (e.g., 2-benzyl-2-(dimethylamino)-4′-morpholinobutyrophenone or 2-methyl-1-[4-(methylthio)phenyl]-2-morpholinopropan-1-one), 4-methyl benzophenone, an azo compound (e.g., 4,4′-Azobis(4-cyanovaleric acid), 1,1′-Azobis(cyclohexanecarbonitrile, Azobisisobutyronitrile, 2,2′-Azobis(2-methylpropionitrile), or 2,2′-Azobis(2-methylpropionitrile)), an inorganic peroxide, an organic peroxide, or any combination thereof. In certain embodiments, the free radical photoinitiator is selected from a benzophenone, a mixture of benzophenone and a tertiary amine containing a carbonyl group which is directly bonded to at least one aromatic ring, and an Irgacure (e.g., Irgacure 907 (2-methyl-1-[4-(methylthio)-phenyl]-2-morpholino-propanone-1) or Irgacure 651 (2,2-dimethoxy-1,2-diphenylethan-1-one). In certain embodiments, the free radical photoinitiator comprises an acetophenone photoinitiator (e.g., 4′-hydroxyacetophenone, 4′-phenoxyacetophenone, 4′-ethoxyaceto-phenone), a benzoin, a benzoin derivative, a benzil, a benzil derivative, a benzophenone (e.g., 4-benzoylbiphenyl, 3,4-(dimethylamino)benzophenone, 2-methylbenzophenone), a cationic photoinitiator (e.g., diphenyliodonium nitrate, (4-iodophenyl)diphenylsulfonium triflate, triphenylsulfonium triflate), an anthraquinone, a quinone (e.g., camphorquinone), a phosphine oxide (e.g., 2,4,6-trimethylbenzoyl diphenyl phosphine oxide (TPO), bis(2,4,6-trimethylbenzoyl)phenyl phosphine oxide (BAPO), ethyl(2,4,6-trimethylbenzoyl)phenyl phosphinate (TPO-L), and bis(2,6-dimethoxybenzoyl)-(2,4,4-trimethylpentyl) phosphine oxide)), a phosphinate, 9,10-phenanthrenequinone, a thioxanthone, any combination thereof, or any derivative thereof. In some embodiments, the composition comprises a photoinitiator comprising SpeedCure TPO-L. In some embodiments, the composition comprises a photoinitiator comprising TPO.

In some embodiment, the photoinitiator is a photoacid initiator such as, for example, aryldiazonium, diaryliodonium, and triarylsulfonium salts.

In some embodiments, the photoinitiator is a photobase generator that generates a base upon exposure to a radiation. In some embodiments, the photobase generator includes photolatent primary, secondary or tertiary amine compound that generates amine upon irradiation. Examples of photolatent primary amines and secondary amines include, but are not limited to, orthonitrobenzylurethane, dimethoxybenzylurethane, benzoins carbamates, O-acyloximes, O-carbamoyl oximes; N-hydroxyimide carbamates; formanilide derivatives; aromatic sulfonamides; cobalt amine complexes and the like. Examples of photolatent tertiary amines include, but are not limited to, α-aminoketone derivatives, α-ammonium ketone derivatives, benzylamine derivatives, benzylammonium salt derivatives, α-aminoalkene derivatives, α-ammonium alkene derivatives. Benzyl ammonium salt derivatives, benzyl substituted amine derivatives, α-amino ketone.

In certain embodiments, the photobase generator comprises 2-(2-nitrophenyl) propyloxycarbonyl-1,1,3,3-tetramethylguanidine (NPPOC-TMG), 2-(2-nitrophenyl)propyl oxycarbonyl-hexylamine (NPPOC-HA), 1-benzyloctahydropyrrolo[1,2-a]pyrimidine, 1-(1-phenylethyl)octahydropyrrolo[1,2-a]pyrimidine, 1-(1-phenylpropyl)octahydropyrrolo[1,2-a]pyrimidine, 1-(1-(o-tolypethyl)octahydropyrrolo[1,2-a]pyrimidine, or 1-(1-(p-tolyl)ethyl)octahydropyrrolo[1,2-a]pyrimidine, or combinations thereof.

In some embodiments, the photoinitiator can have a maximum wavelength absorbance between 200 and 300 nm, between 300 and 400 nm, between 400 and 500 nm, between 500 and 600 nm, between 600 and 700 nm, between 700 and 800 nm, between 800 and 900 nm, between 150 and 200 nm, between 200 and 250 nm, between 250 and 300 nm, between 300 and 350 nm, between 350 and 400 nm, between 400 and 450 nm, between 450 and 500 nm, between 500 and 550 nm, between 550 and 600 nm, between 600 and 650 nm, between 650 and 700 nm, or between 700 and 750 nm. In some embodiments, the photoinitiator has a maximum wavelength absorbance between 300 to 500 nm.

In some embodiments, the initiator further comprises a thermal initiator. In some embodiments, the thermal initiator comprises an azo compound, an inorganic peroxide, an organic peroxide, or any combination thereof. In some embodiments, the thermal initiator is selected from the group consisting of tert-amyl peroxybenzoate, 4,4-azobis(4-cyanovaleric acid), 1,1′-azobis (cyclohexanecarbonitrile), 2,2′-azobisisobutyronitrile (AIBN), benzoyl peroxide, 2,2-bis(tert-butylperoxy)butane, 1,1-bis(tert-butylperoxy)cyclohexane, 2,5-bis(tert-butylperoxy)2,5-dimethylhexane, 2,5-bis(tert-butylperoxy)-2,5-dimethyl-3-hexyne, bis(1-(tert-butylperoxy)-3,3,5-trimethylcyclohexane, tert-butyl hydroxyperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, a derivative thereof, and a combination thereof. In preferred embodiments, the thermal initiator comprises azobisisobutyronitrile, 2,2′-azodi(2-methylbutyronitrile), or combinations thereof. In some embodiments, the thermal initiator is a thermal acid generator or a thermal base generator. Many sulfonium salts can be used as thermal acid generators.

In some embodiments, the curable composition comprises 0.01-10 wt %, 0.02-5 wt %, 0.05-4 wt %, 0.1-3 wt %, 0.1-2 wt %, or 0.1-1 wt %, based on the total weight of the composition, of the initiator. In preferred embodiments, the curable composition comprises 0.1-2 wt %, based on the total weight of the composition, of the initiator. In some embodiments, the curable composition comprises 0.05 to 1 wt %, 0.05 to 2 wt %, 0.05 to 3 wt %, 0.05 to 4 wt %, 0.05 to 5 wt %, 0.1 to 1 wt %, 0.1 to 2 wt %, 0.1 to 3 wt %, 0.1 to 4 wt %, 0.1 to 5 wt %, 0.1 to 6 wt %, 0.1 to 7 wt %, 0.1 to 8 wt %, 0.1 to 9 wt %, or 0.1 to 10 wt %, based on the total weight of the composition, of the photoinitiator. In preferred embodiments, the curable composition comprises 0.1-2 wt % of the photoinitiator. In some embodiments, the curable composition comprises from 0 to 10 wt %, from 0 to 9 wt %, from 0 to 8 wt %, from 0 to 7 wt %, from 0 to 6 wt %, from 0 to 5 wt %, from 0 to 4 wt %, from 0 to 3 wt %, from 0 to 2 wt %, from 0 to 1 wt %, or from 0 to 0.5 wt %, based on the total weight of the composition, of the thermal initiator. In preferred embodiments, the curable composition comprises from 0 to 0.5 wt %, based on the total weight of the composition, of the thermal initiator.

In some embodiments, the curable composition of the present disclosure can comprise one or more polymerizable components in addition to the one or more polymerizable compounds of structure (I), (II) or (III) provided herein. Such one or more polymerizable components can include one or more telechelic oligomers, one or more telechelic polymers, or a combination thereof. In such instances, a telechelic oligomer can have a number-average molecular weight of greater than 500 Da (0.5 kDa) but less than 5 kDa. A telechelic polymer can have a number-average molecular weight of greater than 10 kDa but less than 50 kDa. A telechelic polymer can have a number-average molecular weight of greater than 5 kDa but less than 50 kDa. A telechelic polymer can have a number-average molecular weight of greater than 5 kDa but less than 300 kDa. The telechelic oligomer(s) and/or polymer(s) can comprise reactive functional groups at their termini. In some cases, the reactive functional group can be an acrylate, methacrylate, vinyl acrylate, vinyl methacrylate, allyl ether, silene, alkyne, alkene, vinyl ether, maleimide, fumarate, maleate, itaconate, or styrenyl moiety. In some cases, the reactive functional group can be an acrylate or a methacrylate. A telechelic polymer herein can include polyurethanes, polyesters, block copolymers or any polymers with reactive (e.g., photoreactive and/or polymerizable) end groups. Thus, in various instances, a telechelic block copolymer suitable for the present disclosure is capable of undergoing polymerization and/or photopolymerization with one or more other telechelic polymers, telechelic block copolymers, telechelic oligomers, or telechelic polymers of structure (II) and/or structure (III) provided herein via its terminal monomers. In various cases, the terminal monomers comprise a polymerizable moiety enabling further polymerization reactions. Such polymerization reaction of a telechelic block copolymer with other polymers, oligomers and/or monomers can occur during photo-curing, e.g., in instances where these components are part of a curable composition. In some instances, a telechelic polymer can have one or more glass transition temperatures, wherein at least one glass transition temperature is at 30° C. or lower; preferably 0° C. or lower, even more preferably −20° C. or lower.

In some embodiments, the telechelic oligomer(s) and/or polymer(s) of structure (II), (III) or others, comprise reactive moieties at their termini that comprise photo-reactive moieties. Photoreactive moieties include compounds that photocleave, photocyclize, photodimerize, photoisomerize, photoenolize, photooxidize, photoreduce, photodegrade, photosensitize and other photon assisted reactions. Non-limiting example photoreactive moieties are photoinitiators (see earlier description); photodimering compounds such as coumarins, maleimides, anthracenes, uracils, thymines, and acenapthylenes; and photoisomerization moieties such as azobenzenes, cyclic azobenzenes and azoheteroarenes and derivatives thereof; spiropyran and derivatives thereof; triphenyl methane and derivatives thereof; 4,5-epoxy-2-cyclopentene and derivatives thereof; fulgide and derivatives thereof; thioindigo and derivatives thereof; diarylethene and derivatives thereof; diallylethene and derivatives thereof; and overcrowded alkenes and derivatives thereof.

In some embodiments, the curable composition of the present disclosure can comprise a reactive diluent, a crosslinking modifier, a solvent, a glass transition temperature modifier, a polymerization catalyst, a polymerization inhibitor, a light blocker, a plasticizer, a surface energy modifier, a pigment, a dye, a filler, a biologically significant chemical, or a combination thereof.

In some embodiments, the curable composition of the present disclosure can comprise a reactive diluent homogenously or heterogenously dispersed or patterned therethrough. The degree of heterogenous partitioning (e.g., emulsification) or homogeneity can be controlled with a method or device disclosed herein, for example through agitation prior to printing. In some cases, the degree of heterogeneity in a curable composition can be controlled through addition of solvents or reactive diluents. In various cases, a reactive diluent can comprise an acrylate or methacrylate moiety for incorporation into an oligomeric or polymeric backbone, coupled to a linear or cyclic (e.g., mono-, bi-, or tricyclic) side-chain moiety. Generally, any aliphatic, cycloaliphatic or aromatic molecule with a mono-functional polymerizable reactive functional group can be used (also includes liquid crystalline monomers). In some instances, the polymerizable reactive functional groups are acrylate or methacrylate groups. In some instances, a reactive diluent is a polymerizable compound of structure (I). In some instances, a reactive diluent is a syringol, guaiacol, or vanillin derivative, for example homosalic methacrylate (HSMA), syringyl methacrylate (SMA), isobornyl methacrylate (IBOMA), or isobornyl acrylate (IBOA). In some cases, the reactive diluent used herein can have a low vapor pressure, low viscosity, or a combination thereof. In some embodiments, however, low amounts (e.g., 5% w/w or less) of a reactive diluent may be used. In some embodiments, no reactive diluent is used.

In some embodiments, the curable composition of the present disclosure can comprise a crosslinking modifier. A “crosslinking modifier” as used herein refers to a substance which bonds one oligomer or polymer chain to another oligomer or polymer chain, thereby forming a crosslink. A crosslinking modifier may become part of another substance, such as a crosslink in a polymer material obtained by a polymerization process. In some embodiments, a crosslinking modifier is a curable unit which, when mixed with a curable composition, is incorporated as a crosslink into the polymeric material that results from polymerization of the formulation. In certain embodiments, the curable composition comprises 0-25 wt %, based on the total weight of the composition, of the crosslinking modifier, the crosslinking modifier having a number average molecular weight equal to or less than 3 kDa, equal to or less than 2.5 kDa, equal to or less than 2 kDa, equal to or less than 1.5 kDa, equal to or less than 1.25 kDa, equal to or less than 1 kDa, equal to or less than 800 Da, equal to or less than 600 Da, or equal to or less than 400 Da. In some embodiments, the crosslinking modifier can have a high glass transition temperature (Tg), which leads to a high heat deflection temperature. In some embodiments, the crosslinking modifier has a glass transition temperature greater than −10° C., greater than −5° C., greater than 0° C., greater than 5° C., greater than 10° C., greater than 15° C., greater than 20° C., or greater than 25° C. In some specific embodiments, the curable composition comprises 0-25 wt %, based on the total weight of the composition, of the crosslinking modifier, the crosslinking modifier having a number-average molecular weight equal to or less than 1.5 kDa. In some embodiments, the crosslinking modifier comprises a (meth)acrylate-terminated polyester, a tricyclodecanediol di(meth)acrylate, a vinyl ester-terminated polyester, a tricyclodecanediol vinyl ester, a derivative thereof, or a combination thereof.

In some embodiments, the curable composition of the present disclosure can comprise a solvent. In some embodiments, the solvent comprises a nonpolar solvent. In certain embodiments, the nonpolar solvent comprises pentane, cyclopentane, hexane, cyclohexane, benzene, toluene, 1,4-dioxane, chloroform, diethyl ether, dichloromethane, a derivative thereof, or a combination thereof. In some embodiments, the solvent comprises a polar aprotic solvent. In certain embodiments, the polar aprotic solvent comprises tetrahydrofuran, ethyl acetate, acetone, dimethylformamide, acetonitrile, DMSO, propylene carbonate, a derivative thereof, or a combination thereof. In some embodiments, the solvent comprises a polar protic solvent. In certain embodiments, the polar protic solvent comprises formic acid, n-butanol, isopropyl alcohol, n-propanol, t-butanol, ethanol, methanol, acetic acid, water, a derivative thereof, or a combination thereof. In some embodiments, the curable composition comprises less than 90 wt %, less than 80 wt %, less than 70 wt %, less than 60 wt %, less than 50 wt %, less than 40 wt %, less than 30 wt %, less than 20 et %, less than 15 wt %, less than 10 wt %, less than 5 wt %, less than 3 wt %, less than 2 wt %, or less than 1 wt %, based on the total weight of the composition, of the solvent. In some cases, the solvent is configured to evaporate or separate from the curable reins following curing.

In some embodiments, the curable composition of the present disclosure can comprise a component in addition to a polymerizable compounds of structure (I), (II) or (III) described herein that can alter the glass transition temperature of the cured polymeric material. In such instances, a glass transition temperature modifier (also referred to herein as a Tg modifier or a glass transition modifier) can be present in a curable composition from about 0 to 50 wt %, based on the total weight of the composition. The Tg modifier can have a high glass transition temperature, which leads to a high heat deflection temperature, which can be necessary to use a material at elevated temperatures. In some embodiments, the curable composition comprises 0 to 80 wt %, 0 to 75 wt %, 0 to 70 wt %, 0 to 65 wt %, 0 to 60 wt %, 0 to 55 wt %, 0 to 50 wt %, 1 to 50 wt %, 2 to 50 wt %, 3 to 50 wt %, 4 to 50 wt %, 5 to 50 wt %, 10 to 50 wt %, 15 to 50 wt %, 20 to 50 wt %, 25 to 50 wt %, 30 to 50 wt %, 35 to 50 wt %, 0 to 40 wt %, 1 to 40 wt %, 2 to 40 wt %, 3 to 40 wt %, 4 to 40 wt %, 5 to 40 wt %, 10 to 40 wt %, to 40 wt %, or 20 to 40 wt %, based on the total weight of the composition, of a Tg modifier. In certain embodiments, the curable composition comprises 0-50 wt % of a glass transition modifier. In some instances, the number average molecular weight of the Tg modifier is 0.4 to 5 kDa. In some embodiments, the number average molecular weight of the Tg modifier is from 0.1 to 5 kDa, from 0.2 to 5 kDa, from 0.3 to 5 kDa, from 0.4 to 5 kDa, from 0.5 to 5 kDa, from 0.6 to 5 kDa, from 0.7 to kDa, from 0.8 to 5 kDa, from 0.9 to 5 kDa, from 1.0 to 5 kDa, from 0.1 to 4 kDa, from 0.2 to 4 kDa, from 0.3 to 4 kDa, from 0.4 to 4 kDa, from 0.5 to 4 kDa, from 0.6 to 4 kDa, from 0.7 to 4 kDa, from 0.8 to 4 kDa, from 0.9 to 4 kDa, from 1 to 4 kDa, from 0.1 to 3 kDa, from 0.2 to 3 kDa, from 0.3 to 3 kDa, from 0.4 to 3 kDa, from 0.5 to 3 kDa, from 0.6 to 3 kDa, from 0.7 to 3 kDa, from 0.8 to 3 kDa, from 0.9 to 3 kDa, or from 1 to 3 kDa. A polymerizable compounds of structure (I), (II) or (III) of the present disclosure (which can act by itself as a Tg modifier) and a separate Tg modifier compound can be miscible and compatible in the methods described herein. When used in the subject compositions, the Tg modifier may provide for high Tg and strength values, sometimes at the expense of elongation at break. When such Tg modifiers are used, at least one Tg of the final cured composition can be greater than 40° C., greater than 60° C., greater than 80° C., greater than 100° C., greater than 120° C., greater than 150° C., greater than 200° C., greater than 250° C., or from 40° C. to 100° C., or from 100° C. to 200° C.

In some embodiments, the curable composition of the present disclosure can comprise a polymerization catalyst. In some embodiments, the polymerization catalyst comprises a tin catalyst, a platinum catalyst, a rhodium catalyst, a titanium catalyst, a silicon catalyst, a palladium catalyst, a metal triflate catalyst, a boron catalyst, a bismuth catalyst, a metathesis catalyst (Grubbs catalyst), or combinations thereof. Non-limiting examples of a titanium catalyst include di-n-butylbutoxychlorotin, di-n-butyldiacetoxytin, di-n-butyldilauryltin, dimethyldineodecanoatetin, dioctyldilauryltin, tetramethyltin, and dioctylbis(2-ethylhexylmaleate) tin. Non-limiting examples of a platinum catalyst include platinum-divinyltetramethyl-disiloxane complex, platinum-cyclovinylmethyl-siloxane complex, platinum-octanal complex, and platinum carbonyl cyclovinylmethylsiloxane complex. A non-limiting example of a rhodium catalyst includes tris(dibutylsulfide) rhodium trichloride. Non-limiting examples of a titanium catalyst includes titanium isopropoxide, titanium 2-ethyl-hexoxide, titanium chloride triisopropoxide, titanium ethoxide, and titanium diisopropoxide bis(ethylacetoacetate). Non-limiting examples of a silicon catalyst include tetramethylammonium siloxanolate and tetramethylsilylmethyl-trifluoromethane sulfonate. A non-limiting example of a palladium catalyst includes tetrakis(triphenylphosphine) palladium (0). Non-limiting examples of a metal triflate catalyst include scandium trifluoromethane sulfonate, lanthanum trifluoromethane sulfonate, and ytterbium trifluoromethane sulfonate. A non-limiting example of a boron catalyst includes tris(pentafluorophenyl) boron. Non-limiting examples of a bismuth catalyst include bismuth-zinc neodecanoate, bismuth 2-ethylhexanoate, a metal carboxylate of bismuth and zinc, and a metal carboxylate of bismuth and zirconium. Non-limited examples of a metathesis catalyst include stabilized, late transition metal carbene complex catalysts such as Group VIII transition metal carbene catalysts that include Ru and Os metal carbene catalysts. Examples of Ru carbene catalysts include, but are not limited to, Cp*RuCl(PPh₃)₂ or [Cp*RuCl]₄.

In some embodiments, the curable composition of the present disclosure can comprise a polymerization inhibitor in order to stabilize the composition and prevent premature polymerization. In some embodiments, the polymerization inhibitor is oxygen or oxygen in combination with other compounds such as phenol compounds. In some embodiments, the polymerization inhibitor is a phenolic compound (e.g., butylated hydroxytoluene (BHT)). In some embodiments, the polymerization inhibitor is a stable radical (e.g., 2,2,4,4-tetramethylpiperidinyl-1-oxy radical, 2,2-diphenyl-1-picrylhydrazyl radical, galvinoxyl radical, or triphenylmethyl radical). In some embodiments, more than one polymerization inhibitor is present in the curable composition. In some embodiments, the polymerization inhibitor polymerization inhibitor is an antioxidant, a hindered amine light stabilizer (HAL), a hindered phenol, or a deactivated radical (e.g., a peroxy compound). In some embodiments, the polymerization inhibitor is selected from the group consisting of 4-tert-butylpyrocatechol, tert-butylhydroquinone, 1,4-benzoquinone, 6-tert-butyl-2,4-xylenol, 2-tertbutyl-1,4-benzoquinone, 2,6-di-tert-butyl-p-cresol, 2,6-ditert-butylphenol, 1,1-diphenyl-2-picrylhydrazyl free radical, hydroquinone, 4-methoxyphenol, phenothiazine, derivative thereof, and any combination thereof.

In some embodiments, the curable composition of the present disclosure can comprise a light blocker in order to dissipate UV radiation. In some embodiments, the light blocker absorbs a specific UV energy value and/or range. In some embodiments, the light blocker is a UV light absorber, a pigment, a color concentrate, or an IR light absorber. In some embodiments, the light blocker comprises a benzotriazole (e.g., 2-(2′-hydroxy-phenyl benzotriazole), 2,2-dihydroxy-4-methoxy benzophenone, 9,10-diethoxyanthracene, a hydroxyphenyl triazine, an oxanilide, a benzophenone, or a combination thereof. In some embodiments, the curable composition comprises from 0 to 10 wt %, from 0 to 9 wt %, from 0 to 8 wt %, from 0 to 7 wt %, from 0 to 6 wt %, from 0 to 5 wt %, from 0 to 4 wt %, from 0 to 3 wt %, from 0 to 2 wt %, from 0 to 1 wt %, or from 0 to 0.5 wt %, based on the total weight of the composition, of the light blocker. In more specific embodiments, the curable composition comprises from 0 to 0.5 wt % of the light blocker.

In some embodiments, the curable composition of the present disclosure can comprise a filler. In some embodiments, the filler comprises calcium carbonate (i.e., chalk), kaolin, metakolinite, a kaolinite derivative, magnesium hydroxide (i.e., talc), calcium silicate (i.e., wollastonite), a glass filler (e.g., glass beads, short glass fibers, or long glass fibers), a nanofiller (e.g., nanoplates, nanofibers, or nanoparticles), a silica filler (e.g., a mica, silica gel, fumed silica, or precipitated silica), carbon black, dolomite, barium sulfate, Al(OH)₃, Mg(OH)₂, diatomaceous earth, magnetite, halloysite, zinc oxide, titanium dioxide, cellulose, lignin, a carbon filler (e.g., chopped carbon fiber or carbon fiber), a derivative thereof, or a combination thereof. The filler can be a minor constituent of a curable composition, for example accounting for less than 5 wt %, or can account for a majority of the weight of the curable composition. In some embodiments, the filler is present as at least 0.05 wt %, at least 0.5 wt %, at least 1 wt %, at least 2 wt %, at least 3 wt %, at least 5 wt %, at least 8 wt %, at least 10 wt %, at least 12 wt %, at least 15 wt %, at least 20 wt %, at least 25 wt %, at least 30 wt %, at least 40 wt %, at least 50 wt %, at least 60 wt %, at least 70 wt %, at least 75 wt %, or at least 80 wt % of the curable composition. In some embodiments, the filler is present as at most 80 wt %, at most 75 wt %, at most 70 wt %, at most 60 wt %, at most 50 wt %, at most 40 wt %, at most 30 wt %, at most 25 wt %, at most 20 wt %, at most 15 wt %, at most 10 wt %, at most 8 wt %, at most 5 wt %, at most 3 wt %, at most 2 wt %, at most 1 wt %, or at most 0.5 wt % of the curable composition. In some embodiments, the filler is present between 0.05 and 60 wt %, between 1 and 5 wt %, between 1 and 10 wt %, between 1 and 20 wt %, between 2 and 5 wt %, between 2 and 10 wt %, between 2 and 20 wt %, between 3 and 6 wt %, between 3 and 10 wt %, between 3 and 20 wt %, between 5 and 10 wt %, between 5 and 25 wt %, between 8 and 20 wt %, between 10 and 60 wt %, between 12 and 25 wt %, between 15 and 30 wt %, between 15 and 40 wt %, between 20 and 35 wt %, between 25 and 50 wt %, between 30 and 50 wt %, between 35 and 65 wt %, between 40 and 65 wt %, between 40 and 80 wt %, between 50 and 75 wt %, or between 60 and 80 wt % of the curable composition. In some embodiments, the filler is present between 10 and 60 wt % of the curable composition. In some embodiments, the filler is present between 20 and 60 wt % of the curable composition. In some embodiments, the filler is present between 20 and 40 wt % of the curable composition. In some embodiments, the filler is present between 30 and 50 wt % of the curable composition.

In some embodiments, the curable composition of the present disclosure can comprise a pigment, a dye, or a combination thereof. A pigment is typically a suspended solid that may be insoluble in the resin. A dye is typically dissolved in the curable composition. In some embodiments, the pigment comprises an inorganic pigment. In some embodiments, the inorganic pigment comprises an iron oxide, barium sulfide, zinc oxide, antimony trioxide, a yellow iron oxide, a red iron oxide, ferric ammonium ferrocyanide, chrome yellow, carbon black, or aluminum flake. In some embodiments, the pigment comprises an organic pigment. In some embodiments, the organic pigment comprises an azo pigment, an anthraquinone pigment, a copper phthalocyanine (CPC) pigment (e.g., phthalo blue or phthalo green) or a combination thereof. In some embodiments, the dye comprises an azo dye (e.g., a diarylide or Sudan stain), an anthraquinone (e.g., Oil Blue A or Disperse Red 11), or a combination thereof. In some embodiments, the curable composition comprises from about 0.001 to about 3 wt %, based on the total weight of the composition, of the pigment. In some embodiments, the curable composition comprises from about 0.005 to about 2 wt %, based on the total weight of the composition, of the pigment. In some cases, the curable composition comprises from about 0.005 to about 0.5 wt %, based on the total weight of the composition, of the pigment. In some embodiments, the curable composition comprises from about 0.01 to about 0.3 wt %, based on the total weight of the composition, of the pigment. In some embodiments, the curable composition comprises from about 0.005 to about 0.1 wt %, based on the total weight of the composition, of the pigment.

In some embodiments, the curable composition of the present disclosure can comprise a surface energy modifier. In some embodiments, the surface energy modifier can aid the process of releasing a polymer from a mold. In some embodiments, the surface energy modifier can act as an antifoaming agent. In some embodiments, the surface energy modifier comprises a defoaming agent, a deairation agent, a hydrophobization agent, a leveling agent, a wetting agent, or an agent to adjust the flow properties of the curable composition. In some embodiments, the surface energy modifier comprises an alkoxylated surfactant, a silicone surfactant, a sulfosuccinate, a fluorinated polyacrylate, a fluoropolymer, a silicone, a star-shaped polymer, an organomodified silicone, or any combination thereof. In some embodiments, the curable composition comprises from between about 0.01 to about 3 wt % of the surface energy modifier. In some embodiments, the curable composition comprises from about 0.05 to about 1.5 wt %, from about 0.1 to about 1.5 wt %, from about 0.3 to about 1.5 wt %, from about 0.1 to about 1 wt %, from about 0.1 to about 0.5 wt %, from about 0.2 to about 1 wt %, from about 0.3 to about 0.7 wt %, or from about 0.4 to about 1 wt %, based on the total weight of the composition, of the surface energy modifier.

In some embodiments, the curable composition of the present disclosure can comprise a plasticizer. A plasticizer can be a nonvolatile material that can reduce interactions between polymer chains, which can decrease glass transition temperature, melt viscosity, and elastic modulus. In some embodiments, the plasticizer comprises a dicarboxylic ester plasticizer, a tricarboxylic ester plasticizer, a trimellitate, an adipate, a sebacate, a maleate, or a bio-based plasticizer. In some embodiments, the plasticizer comprises a dicarboxylic ester or a tricarboxylic ester comprising a dibasic ester, a phthalate, bis(2-ethylhexyl) phthalate (DEHP), bis(2-propylheptyl) phthalate (DPHP), diisononyl phthalate (DINP), di-n-butyl phthalate (DBP), butyl benzyl phthalate (BBZP), diisodecyl phthalate (DIDP), dioctyl phthalate (DOP), diisooctyl phthalate (DIOP), diethyl phthalate (DEP), diisobutyl phthalate (DIBP), di-n-hexyl phthalate, a derivative thereof, or a combination thereof. In some embodiments, the plasticizer comprises a trimellitate comprising trimethyl trimellitate (TMTM), tri-(2-ethylhexyl) trimellitate (TEHTM), tri-(n-octyl, n-decyl) trimellitate (ATM), tri(heptyl, nonyl) trimellitate (LTM), n-octyl trimellitate (OTM), trioctyl trimellitate, a derivative thereof, or a combination thereof. In some embodiments, the plasticizer comprises an adipate comprising bis(2-ethylhexyl) adipate (DEHA), dimethyl adipate (DMAD), monomethyl adipate (MMAD), dioctyl adipate (DOA), Bis[2-(2-butoxyethoxy) ethyl] adipate, dibutyl adipate, diisobutyl adipate, diisodecyl adipate, a derivative thereof, or a combination thereof. In some embodiments, the plasticizer comprises a sebacate comprising dibutyl sebacate (DBS), Bis(2-ethylhexyl) sebacate, diethyl sebacate, dimethyl sebacate, a derivative thereof, or a combination thereof. In some embodiments, the plasticizer comprises a maleate comprises Bis(2-ethyl-hexyl) maleate, dibutyl maleate, diisobutyl maleate, a derivative thereof, or a combination thereof. In some embodiments, the plasticizer comprises a bio-based plasticizer comprising an acetylated monoglyceride, an alkylcitrate, a methyl ricinoleate, or a green plasticizer. In some embodiments, the alkyl citrate is selected from the group consisting of triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, a derivative thereof, or a combination thereof. In some embodiments, the green plasticizer is selected from the group consisting of epoxidized soybean oil, epoxidized vegetable oil, epoxidized esters of soybean oil, a derivative thereof, or a combination thereof. In some embodiments, the plasticizer comprises an azelate, a benzoate (e.g., sucrose benzoate), a terephthalate (e.g., dioctyl terephthalate), 1, 2-cyclohexane dicarboxylic acid diisononyl ester, alkyl sulphonic acid phenyl ester, a sulfonamide (e.g., N-ethyl toluene sulfonamide, N-(2-hydroxy propyl) benzene sulfonamide, N-(n-butyl) benzene sulfonamaide), an organophosphate (e.g., tricresyl phosphate or tributyl phosphate), a glycol (e.g., triethylene glycol dihexanoate or tetraethylene glycol diheptanoate), a polyether, polybutene, a derivative thereof, or a combination thereof.

In some embodiments, the curable composition of the present disclosure can comprise a biologically significant chemical. In some embodiments, the biologically significant chemical comprises a hormone, an enzyme, an active pharmaceutical ingredient, an antibody, a protein, a drug, or any combination thereof. In some embodiments, the biologically significant chemical comprises a pharmaceutical composition, a chemical, a gene, a polypeptide, an enzyme, a biomarker, a dye, a compliance indicator, an antibiotic, an analgesic, a medical grade drug, a chemical agent, a bioactive agent, an antibacterial, an antibiotic, an anti-inflammatory agent, an immune-suppressive agent, an immune-stimulatory agent, a dentinal desensitizer, an odor masking agent, an immune reagent, an anesthetic, a nutritional agent, an antioxidant, a lipopolysaccharide complexing agent or a peroxide.

In some embodiments, the added component (e.g., a crosslinking modifier, a glass transition temperature modifier, a toughness modifier, a polymerization catalyst, a polymerization inhibitor, a light blocker, a plasticizer, a solvent, a surface energy modifier, a pigment, a dye, a filler, or a biologically significant chemical) is functionalized so that it can be incorporated into the polymeric material so that it cannot readily be extracted from the final cured material. In certain embodiments, the polymerization catalyst, polymerization inhibitor, light blocker, plasticizer, surface energy modifier, pigment, dye, and/or filler, are functionalized to facilitate their incorporation into the cured polymeric material.

### Curable Composition Properties

Curable (e.g., photo-curable) compositions herein can be characterized by having one or more properties. In some embodiments, a polymerizable compound having any one of structures (I), (II) or (III) can reduce a viscosity of the curable composition by at least about 5% compared to a curable composition that does not comprise such polymerizable compounds, thereby providing improved printing conditions compared to existing resins used in additive manufacturing. In some instances, the viscosity of the curable composition of the present disclosure can be reduced by at least about 10%, 20%, 30%, 40%, or 50%. In some instances, a curable composition of the present disclosure can have a viscosity from about 30 cP to about 50,000 cP at a printing temperature. In some embodiments, the curable composition has a viscosity less than or equal to 30,000 cP, less than or equal to 25,000 cP, less than or equal to 20,000 cP, less than or equal to 19,000 cP, less than or equal to 18,000 cP, less than or equal to 17,000 cP, less than or equal to 16,000 cP, less than or equal to 15,000 cP, less than or equal to 14,000 cP, less than or equal to 13,000 cP, less than or equal to 12,000 cP, less than or equal to 11,000 cP, less than or equal to 10,000 cP, less than or equal to 9,000 cP, less than or equal to 8,000 cP, less than or equal to 7,000 cP, less than or equal to 6,000 cP, or less than or equal to 5,000 cP at 25° C. In some embodiments, the curable composition has a viscosity less than 15,000 cP at 25° C. In some embodiments, the photo-curable resin has a viscosity less than or equal to 100,000 cP, less than or equal to 90,000 cP, less than or equal to 80,000 cP, less than or equal to 70,000 cP, less than or equal to 60,000 cP, less than or equal to 50,000 cP, less than or equal to 40,000 cP, less than or equal to 35,000 cP, less than or equal to 30,000 cP, less than or equal to 25,000 cP, less than or equal to 20,000 cP, less than or equal to 15,000 cP, less than or equal to 10,000 cP, less than or equal to 5,000 cP, less than or equal to 4,000 cP, less than or equal to 3,000 cP, less than or equal to 2,000 cP, less than or equal to 1,000 cP, less than or equal to 750 cP, less than or equal to 500 cP, less than or equal to 250 cP, less than or equal to 100 cP, less than or equal to 90 cP, less than or equal to 80 cP, less than or equal to 70 cP, less than or equal to 60 cP, less than or equal to 50 cP, less than or equal to 40 cP, less than or equal to 30 cP, less than or equal to 20 cP, or less than or equal to 10 cP at a printing temperature. In some embodiments, the curable composition has a viscosity from 50,000 cP to 30 cP, from 40,000 cP to 30 cP, from 30,000 cP to 30 cP, from 20,000 cP to 30 cP, from 10,000 cP to 30 cP, or from 5,000 cP to 30 cP at a printing temperature. In some embodiments, the printing temperature is from 0° C. to 25° C., from 25° C. to 40° C., from 40° C. to 100° C., or from 20° C. to 150° C. In some embodiments, the curable composition has a viscosity from 30 cP to 50,000 cP at a printing temperature, wherein the printing temperature is from 20° C. to 150° C. In yet other embodiments, the curable composition has a viscosity less than 20,000 cP at a print temperature. In some embodiments, the printing temperature is at least about 20° C., 25° C., 30° C., 40° C., 50° C., 60° C., 80° C., or 100° C. In some embodiments, the print temperature is from 25° C. to 150° C., from 25° C. to 120° C., from 25° C. to 115° C., or from 30° C. to 100° C. In preferred embodiments, the print temperature is from 25° C. to 120° C. In some embodiments, the curable composition is a solid at room temperature (meaning the curable composition does not flow in any time periods, such as days, months, or years).

In some embodiments, the curable composition herein has a melting temperature greater than room temperature. In some embodiments, the curable composition has a melting temperature greater than 20° C., greater than 25° C., greater than 30° C., greater than 35° C., greater than 40° C., greater than 45° C. greater than 50° C., greater than 55° C., greater than 60° C., greater than 65° C., greater than 70° C., greater than 75° C., or greater than 80° C. In some embodiments, the curable composition has a melting temperature from 20° C. to 250° C., from 30° C. to 180° C., from 40° C. to 160° C., or from 50° C. to 140° C. In some embodiments, the curable composition has a melting temperature greater than 60° C. In other embodiments, the curable composition has a melting temperature from 80° C. to 110° C. In some instances, a curable composition can have a melting temperature of about 80° C. before polymerization, and after polymerization, the resulting polymeric material can have a melting temperature of about 100° C.

In certain instances, it may be advantageous that a curable composition is in a liquid phase at an elevated temperature. As an example, a conventional curable composition can comprise polymerizable components that may be viscous at a process temperature, and thus can be difficult to use in the fabrication of objects (e.g., using 3D printing). As a solution for that technical problem, the present disclosure provides curable compositions comprising polymerizable compounds of any one of structures (I), (II) or (III) described herein that can melt at an elevated temperature, e.g., at a temperature of fabrication (e.g., during 3D printing), and can have a decreased viscosity at the elevated temperature, which can make such curable composition more applicable and usable for uses such as 3D printing. Hence, in some embodiments, provided herein are curable compositions that are a liquid at an elevated temperature. In some embodiments, the elevated temperature is at or above the melting temperature (Tₘ) of the curable composition. In certain embodiments, the elevated temperature is a temperature in the range from about 40° C. to about 100° C., from about 60° C. to about 100° C., from about 80° C. to about 100° C., or from about 40° C. to about 120° C. In some embodiments, the elevated temperature is a temperature above about 40° C., above about 60° C., above about 80° C., or above about 100° C. In some embodiments, a curable composition herein is a liquid at an elevated temperature with a viscosity less than about 50 Pa·s, less than about 20 Pa·s, less than about 10 Pa·s, less than about 5 Pa·s, or less than about 1 Pa·s. In some embodiments, a curable resin herein is a liquid at an elevated temperature of above about 40° C. with a viscosity less than about 20 Pa·s. In yet other embodiments, a photo-curable resin herein is a liquid at an elevated temperature of above about 40° C. with a viscosity less than about 1 Pa·s.

In some embodiments, at least a portion of a curable composition herein has a melting temperature below about 100° C., below about 90° C., below about 80° C., below about 70° C., or below about 60° C. In some embodiments, at least a portion of a curable composition herein melts at an elevated temperature between about 100° C. and about 20° C., between about 90° C. and about 20° C., between about 80° C. and about 20° C., between about 70° C. and about 20° C., between about 60° C. and about 20° C., between about 60° C. and about 10° C., or between about 60° C. and about 0° C.

The curable composition can, in some embodiments, be characterized by a low crystalline content when the curable composition is at an elevated temperature (e.g., during the 3D printing process). The low crystalline content can be due, e.g., to the elevated temperature being above the melting temperature of the crystalline phases. In some embodiments, the curable composition has less than 60% crystalline content, less than 50% crystalline content, less than 50% crystalline content, less than 40% crystalline content, less than 20% crystalline content, less than 10% crystalline content, or less than 5% crystalline content at the print temperature, as measured by X-ray diffraction. The print temperature can be a temperature from 20-120° C. In some embodiments, at least 90% of the polymerizable compounds of structure (I), (II) or (III) herein is in a liquid phase at 90° C. In some embodiments, the curable composition is a liquid with no crystallinity at the printing temperature and before curing, but may become crystalline during or after curing, and/or when cooling from the cure temperature. When the curable composition has no crystallinity, the curable composition can be less viscous. In some embodiments, it is preferred to have the viscosity as low as possible. In other embodiments, it is advantageous to have a desired degree of crystallinity present at the printing temperature. For example, a small amount of crystallinity can facilitate the crystallization process either during printing, or upon cooling down (e.g., they can act as crystallization seeds) and/or improve the strength of the partially cured composition.

The curable composition of the present disclosure can comprise less than about 20 wt % or less than about 10 wt % hydrogen bonding units. In some embodiments, a curable composition herein comprises less than about 15 wt %, less than about 10 wt %, less than about 9 wt %, less than about 8 wt %, less than about 7 wt %, less than about 6 wt %, less than about 5 wt %, less than about 4 wt %, less than about 3 wt %, less than about 2 wt %, or less than about 1 wt % hydrogen bonding units, wherein wt % is the weight percent of species, including monomeric units in polymerized, oligomerized, and monomeric form, capable of forming at least one hydrogen bond.

### Polymeric Materials

The present disclosure provides polymeric materials generated by curing the curable composition described herein (also referred herein as “printed polymeric materials” and “cured polymeric materials”). The cured polymeric materials comprise a crystalline domain (also referred to herein as a “crystalline phase”) and an amorphous domain (also referred to herein as an “amorphous phase”).

In some embodiments, the polymeric material has a melting temperature (Tm) above 20° C., above 30° C., above 40° C., above 50° C., above 60° C., or above 70° C., as measured by DSC. In some embodiments, the use temperature is different from temperatures near standard room temperatures, and the polymeric material has a melting temperature greater than or equal to 10° C., greater than or equal to 30° C., greater than or equal to 60° C., greater or equal to 80° C., greater than or equal to 100° C., or greater than or equal to 150° C. above the use temperature. In preferred embodiments, the polymeric material has a melting temperature greater than 60° C. In some embodiments, the polymeric material has a melting temperature between 60° C. and 180° C., between 60° C. and 120° C., or between 70° C. and 100° C.

In some embodiments, the polymeric material has a glass transition temperature (Tg) less than 80° C., less than 70° C., less than 60° C., less than 50° C., less than 40° C., less than 30° C., less than 20° C., less than 10° C., less than 0° C., less than −10° C., less than 15° C., less than −20° C., less than −40° C., as measured by DSC. In some embodiments, the polymeric material may have more than one glass transition temperature. For example, in some embodiments, the polymeric material has a first glass transition temperature less than 40° C. and a second glass transition temperature greater than 60° C. In preferred embodiments, the polymeric material has an onset temperature at or below the use temperature.

In some embodiments, the polymeric material has a glass transition temperature, a melting temperature, and a crystallization temperature. In some embodiments, the polymeric material has a glass transition temperature below 40° C., below 0° C., below −15° C., or below −40° C., and a melting temperature greater than 40° C., greater than 80° C., greater than 100° C., greater than 180° C., and greater than 200° C.

In some embodiments, the polymeric material comprises at least one crystalline domain and an amorphous domain. In some embodiments, the crystalline domain may be a liquid crystalline domain. The combination of these two domains can create a polymeric material that has a high modulus phase and a low modulus phase. By having these two phases, the polymeric material can have high modulus and high elongation, as well as high stress remaining following stress relaxation.

### Phase Separation in Polymeric Materials

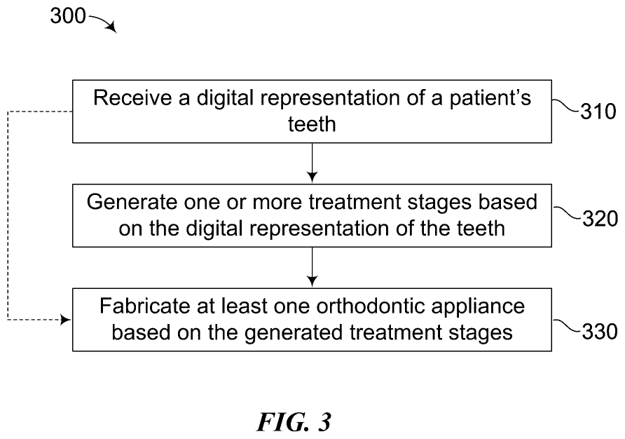

In some embodiments herein, the curable composition herein can be cured by exposing such composition to electromagnetic radiation of appropriate wavelength. Such curing or polymerization can induce phase separation in the forming of polymeric material. Such polymerization-induced phase separation can occur along one or more lateral and vertical direction(s) (see, e.g., FIG. 5). Polymerization-induced phase separation can generate one or more polymeric phases in the resulting polymeric material. A curable composition undergoing polymerization and polymerization-induced phase separation can comprise one or more polymerizable compounds or monomers of the present disclosure. Thus, in some cases, at least one polymeric phase of the one or more polymeric phases generated during curing and present in the resulting polymeric material can comprise, in a polymerized form, at least one of the one or more polymerizable compounds of the present disclosure. In an example, a photo-curable composition comprising a polymerizable compound of structure (I), (II) or (III) is cured by exposure to electromagnetic radiation of appropriate wavelength.

A polymeric phase of a polymeric material of the present disclosure can have a certain size or volume. In some embodiments, a polymeric phase is 3-dimensional, and can have at least one dimension with less than 1000 μm, less than 500 μm, less than 250 μm, less than 200 μm, less than 150 μm, less than 100 μm, less than 90 μm, less than 80 μm, less than 70 μm, less than 60 μm, less than 50 μm, less than 40 μm, less than 30 μm, less than 20 μm, or less than 10 μm. In certain embodiments, the polymeric phase can have at least two dimensions with less than 1000 μm, less than 500 μm, less than 250 μm, less than 200 μm, less than 150 μm, less than 100 μm, less than 90 μm, less than 80 μm, less than 70 μm, less than 60 μm, less than 50 μm, less than 40 μm, less than 30 μm, less than 20 μm, or less than 10 μm. In certain embodiments, the polymeric phase can have three dimensions with less than 1000 μm, less than 500 μm, less than 250 μm, less than 200 μm, less than 150 μm, less than 100 μm, less than 90 μm, less than 80 μm, less than 70 μm, less than 60 μm, less than 50 μm, less than 40 μm, less than 30 μm, less than 20 μm, or less than 10 μm. In some embodiments, a polymeric material comprises an average polymeric phase size of less than about 5 μm in at least one spatial dimension.

In various aspects, the present disclosure provides a polymeric material that can comprise one or more polymeric phases, wherein at least one polymeric phase of the one or more polymeric phases is a crystalline phase. In various aspects, the present disclosure provides a polymeric material that can comprise one or more polymeric phases, wherein at least one polymeric phase of the one or more polymeric phases is an amorphous phase. In some instances, provided herein is a polymeric material that can comprise two or more polymeric phases, wherein at least one polymeric phase of the one or more polymeric phases is a crystalline phase, and at least one polymeric phase of the one or more polymeric phases an amorphous phase.

Hence, in some instance, provided herein is a polymeric material comprising: (i) at least one crystalline phase comprising at least one polymer crystal having a melting temperature above 20° C.; and (ii) at least one amorphous phase comprising at least one amorphous polymer having a glass transition temperature greater than 40° C. In some cases, the at least one crystalline phase can comprise, in a polymerized form, one or more polymerizable compounds of the present disclosure. In some aspects, the at least one polymer crystal has a melting temperature above 30° C., 40° C., 50° C., 60° C., or above 70° C. In some cases, the at least one amorphous phase can comprise, in a polymerized form, one or more polymerizable compounds of the present disclosure. In some aspects, such amorphous phase has a glass transition temperature greater than 50° C., 60° C., 70° C., 80° C., 90° C., 100° C. or greater than 110° C.

### Amorphous Polymeric Phases

The present disclosure provides polymeric materials comprising one or more amorphous phases, e.g., generated by polymerization-induced phase separation. Such polymeric materials, or regions of such material that contain polymeric phases, can provide fast response times to external stimuli, which can confer favorable properties to the polymeric material comprising the crystalline phase and/or the amorphous phase, e.g., for using the polymeric material in a medical device (e.g., an orthodontic appliance). In some cases, a polymeric material comprising one or more amorphous polymeric phases can, for example, provide flexibility to the cured polymeric material, which can increase its durability (e.g., the material can be stretched or bent while retaining its structure, while a similar material without amorphous phases can crack). In certain embodiments, amorphous phases can be characterized by randomly oriented polymer chains (e.g., not stacked in parallel or in crystalline structures). In some embodiments, such amorphous phase of a polymeric material can have a glass transition temperature of greater than about 10° C., 20° C., 30° C., 40° C., 50° C., 60° C., 70° C., 80° C., 90° C., 100° C., or greater than about 110° C. In some embodiments, an amorphous phase can have a glass transition temperature from about 40° C. to about 60° C., from about 50° C. to about 70° C., from about 60° C. to about 80° C., or from about 80° C. to about 110° C. In some embodiments, the amorphous phase has a glass transition temperature less than 10° C., 0° C., −10° C., −30° C., −50° C. In some preferred aspects, one or more amorphous phases will have a glass transition temperature less than 0° C. In some embodiments, two or more amorphous phases have glass transition temperatures above 60° C. and below 10° C.

In some embodiments, an amorphous phase herein (also referred to herein as an amorphous domain) can comprise at least about 5%, 10%, 20%, 30%, 40%, 50%, 60%, 70%, 80% or at least about 90% amorphous polymeric material in an amorphous state. The percentage of amorphous polymeric material in an amorphous phase generally refers to total volume percent.

In some embodiments, an amorphous polymeric phase can comprise one or more polymer types that may have formed, during curing, from polymerizable compounds of structure (I), (II) or (III), and any other polymerizable components that may have been present in the curable composition used to produce the polymeric material that contains the amorphous polymeric phase.

In some instances, polymerizable components of a curable composition that can form a crystalline material, can form an amorphous phase instead when exposed to conditions that prevent their crystallization. Hence, in some cases, materials that may conventionally be considered crystalline can be used as amorphous material. As a non-limiting example, polycaprolactone can be a crystalline polymer, but when mixed with other polymerizable monomers and telechelic polymers, crystal formation may be prevented and an amorphous phase can form.

### Crystalline Polymeric Phases

As further described herein, a polymeric material of the present disclosure can comprise one or more crystalline phases, e.g., generated by polymerization-induced phase separation during curing. As described herein, a crystalline phase is a polymeric phase of a cured polymeric material that comprises at least one polymer crystal. As disclosed herein, a crystalline phase may consist of a single polymeric crystal, or may comprise a plurality of polymeric crystals.

In some embodiments, a crystalline polymeric phase can have a melting temperature equal to or greater than about 20° C., 30° C., 40° C., 50° C., 60° C., 70° C., 80° C., 90° C., 100° C., 120° C., or equal to or greater than about 150° C. In some cases, at least two crystalline phases of a plurality of crystalline phases can have a different melting temperature due to, e.g., differences in crystalline phase sizes, impurities, degree of cross-linking, chain lengths, thermal history, rates at which polymerization occurred, degree of phase separation, or any combination thereof. In some embodiments, at least two crystalline phases of a polymeric material can each have a polymer crystal melting temperature within about 5° C. of each other. In some instances, such melting temperature difference can be less than about 5° C. In other instances, such melting temperature difference can be greater than about 5° C. In some embodiments, each of the polymer crystal melting temperatures of a polymeric material can be from about 40° C. to about 120° C. In some embodiments, at least about 80% of the crystalline domains of a polymeric material can comprise a polymer crystal having a melting temperature between about 40° C. and about 120° C.

In some embodiments, at least 80% of the crystalline phases have a crystal melting point at a temperature between 0° C. and 120° C. In some embodiments, at least 80% of the crystalline phases have a crystal melting point at a temperature between 40° C. and 60° C., between 40° C. and 80° C., between 40° C. and 120° C., between 60° C. and 80° C., between 60° C. and 120° C., between 80° C. and 120° C., or greater than 120° C. In some embodiments, at least 90% of the crystalline phases have a crystal melting point at a temperature between 0° C. and 120° C. In some embodiments, at least 90% of the crystalline phases have a crystal melting point at a temperature between 40° C. and 60° C., between 40° C. and 80° C., between 40° C. and 100° C., between 60° C. and 80° C., between 60° C. and 120° C., between 80° C. and 120° C., or greater than 120° C. In some embodiments, at least 95% of the crystalline phases have a crystal melting point at a temperature between 0° C. and 120° C. In some embodiments, at least 95% of the crystalline phases have a crystal melting point at a temperature between 40° C. and 60° C., between 40° C. and 80° C., between 40° C. and 120° C., between 60° C. and 80° C., between 60° C. and 120° C., between 80° C. and 120° C., or greater than 120° C.

In certain embodiments, the temperature at which a crystalline phase of a cured polymeric material melts can be controlled, e.g., by using different amounts and types of polymerizable components in the curable resin.

In some embodiments, the curing of a resin can occur at an elevated temperature (e.g., at about 90° C.), and as the cured polymeric material cools to room temperature (e.g., 25° C.), the cooling can trigger the formation and/or growth of polymeric crystals in the polymeric material. In some instances, a polymeric material can be a solid at room temperature and can be crystalline-free, but can form crystalline phase over time. In such cases, a crystalline phase can form within 1 hour, within 2 hours, within 4 hours, within 8 hours, within 12 hours, within 18 hours, within 1 day, within 2 days, within 3 days, within 4 days, within 5 days, within 6 days, or within 7 days after cooling. In some embodiments, a crystalline phase can form while the cured polymeric material is in a cooled environment, e.g., an environment having a temperature from about 40° C. to about 30° C., from about 30° C. to about 20° C., from about 20° C. to about 10° C., from about 10° C. to about 0° C., from about 0° C. to about −10° C., from about −10° C. to about −20° C., from about −20° C. to about −30° C., or below about −30° C. In some instances, a polymeric material can be heated to an elevated temperature in order to induce crystallization or formation of crystalline phases. As a non-limiting example, a polymeric material that is near its glass transition temperature can comprise polymer chains that may not be mobile enough to organize into crystals, and thus further heating the material can increase chain mobility and induce formation of crystals.

In some embodiments, the generation, formation, and/or growth of a polymeric phase is spontaneous. In some embodiments, the generation, formation, and/or growth of a polymer crystal is facilitated by a trigger. In some embodiments, the trigger comprises the addition of a seeding particle (also referred to herein as a “seed”), which can induce crystallization. Such seeds can include, for example, finely ground solid material that has at least some properties similar to the forming crystals. In some embodiments, the trigger comprises a reduction of temperature. In certain embodiments, the reduction of temperature can include cooling the cured material to a temperature from 40° C. to 30° C., from 30° C. to 20° C., from 20° C. to 10° C., from 10° C. to 0° C., from 0° C. to −10° C., from −10° C. to −20° C., from −20° C. to −30° C., or below −30° C. In some embodiments, the trigger can comprise an increase in temperature. In certain embodiments, the increase of temperature can include heating the polymeric cured material to a temperature from 20° C. to 40° C., from 40° C. to 60° C., from 60° C. to 80° C., from 80° C. to 100° C., or above 100° C. In some embodiments, the trigger comprises a force placed on the cured polymeric material. In certain embodiments, the force includes squeezing, compacting, pulling, twisting, or providing any other physical force to the material. In some embodiments, the trigger comprises an electrical charge and/or electrical field applied to the material. In some embodiments, formation of one or more crystalline phases may be induced by more than one trigger (i.e., more than one type of trigger can facilitate the generation, formation, and/or growth of crystals). In some embodiments, the polymeric material comprises a plurality of crystalline phases, and at least two of the crystalline phases may be induced by different triggers.

In some embodiments, a polymeric material herein comprises a crystalline phase that has discontinuous phase transitions (e.g., first-order phase transitions). In some cases, a polymeric material has discontinuous phase transitions, due at least in part to the presence of one or more crystalline domains. As a non-limiting example, a cured polymeric material comprising one or more crystalline domains can, when heated to an elevated temperature, have one or more portions that melt at such elevated temperature, as well as one or more portions that remain solid.

In some embodiments, a cured polymeric material comprises crystalline phases that are continuous and/or discontinuous phases. A continuous phase can be a phase that can be traced or is connected from one side of a polymeric material to another side of the material; for instance, a closed-cell foam has material comprising the foam that can be traced across the sample, whereas the closed cells (bubbles) represent a discontinuous phase of air pockets. In some embodiments, the at least one crystalline phase forms a continuous phase while the at least one amorphous phase is discontinuous across the material. In another embodiment, the at least one crystalline phase is discontinuous and the at least one amorphous phase is continuous across the material. In another embodiment, both the at least one crystalline and the at least one amorphous phases are continuous across the material. In some embodiments, a polymeric material comprises a plurality of crystalline phases, wherein one or more crystalline phases of the plurality of crystalline phases have a high melting point (e.g., at least about 50° C., 70° C., or 90° C.) and are in a discontinuous phase, while another one or more crystalline phases of the plurality of crystalline phases have a low melting point (e.g., at less than about 50° C., 70° C., or 90° C.) and are in a continuous phase. In some embodiments, two continuous amorphous phases are present. In other embodiments, one continuous and one discontinuous amorphous phase is present

In some embodiments, a polymeric material comprises an average crystalline phase size of less than about 100 μm, 50 μm, 20 μm, 10 μm, or less then about 5 μm in at least one spatial dimension.

In some embodiments, a polymer crystal of a crystalline phase can comprise greater than about 40 wt %, greater than about 50 wt %, greater than about 60 wt %, greater than about 70 wt %, greater than about 80 wt %, or greater than about 90 wt % of linear polymers and/or linear oligomers.

In some embodiments, the polymeric material has a crystalline content (i.e., the volume percentage of polymer crystals) from 20% to 60% by volume. In some embodiments, the crystalline content is between 30% and 50%, or between 50% and 80%. The crystalline content can be measured by X-ray diffraction. In some embodiments, a polymeric material herein can comprise a weight ratio of crystalline phases to amorphous phases from about 1:99 to about 99:1.

In some embodiments, a cured polymer such as a crosslinked polymer, can be characterized by a tensile stress-strain curve that displays a yield point after which the test specimen continues to elongate, but there is no (detectable) or only a very low increase in stress. Such yield point behavior can occur “near” the glass transition temperature, where the material is between the glassy and rubbery regimes and may be characterized as having viscoelastic behavior. In some embodiments, viscoelastic behavior is observed in the temperature range from about 20° C. to about 40° C. The yield stress is determined at the yield point. In some embodiments, the modulus is determined from the initial slope of the stress-strain curve or as the secant modulus at 1% strain (e.g., when there is no linear portion of the stress-strain curve). The elongation at yield is determined from the strain at the yield point. When the yield point occurs at a maximum in the stress, the ultimate tensile strength is less than the yield strength. For a tensile test specimen, the strain is defined by ln (l/l0), which may be approximated by (l−l0)/l0 at small strains (e.g., less than approximately 10%) and the elongation is l/l0, where l is the gauge length after some deformation has occurred and l0 is the initial gauge length. The mechanical properties can depend on the temperature at which they are measured. The test temperature may be below the expected use temperature for an orthodontic appliance such as 35° C. to 40° C. In embodiments, the test temperature is 23±2° C.

As provided further herein, the polymeric material comprising a crystalline phase (can also referred to herein as a crystalline domain) and an amorphous phase (can also referred to herein as an amorphous domain) can have improved characteristics, such as the ability to act quickly (e.g., vibrate quickly and react upon application of strain, from the elastic characteristics of the amorphous domain) and also provide strong modulus (e.g., are stiff and provide strength, from the crystalline domain). The polymer crystals disclosed herein can comprise closely stacked and/or packed polymer chains. In some embodiments, the polymer crystals comprise long oligomer or long polymer chains that are stacked in an organized fashion, overlapping in parallel. The polymer crystals can in some cases be pulled out of a crystalline phase, resulting in an elongation as the polymer chains of the polymer crystal are pulled (e.g., application of a force can pull the long polymer chain of the polymer crystal, thus introducing disorder to the stacked chains, pulling at least a portion out of its crystalline state without breaking the polymer chain). This is in contrast with fillers that are traditionally used in the formation of resins for materials with high flexural modulus, which can simply slip through the amorphous phase as forces are applied to the polymeric material or when the fillers are covalently bonded to the polymers causing a reduction in the elongation to break for the material. The use of polymer crystals in the resulting polymeric material can thus provide a less brittle product that can retain more of the original physical properties following use (i.e., are more durable), and retains elastic characteristics through the combination of amorphous and crystalline phases.

In some embodiments, the polymer crystals comprise liquid crystalline. The liquid crystalline domains may form different morphologies depending on the temperature. The liquid crystalline moieties are selected so that the liquid crystalline domain in the resulting polymeric material can comprise an ordered lattice (e.g., a liquid crystal) when at a temperature from 20° C. to 250° C., from 30° C. to 180° C., from 40° C. to 160° C., or from 50° C. to 140° C., or comprise a disordered (e.g., not crystalline) morphology when at a temperature greater than 50° C., greater than 60° C., greater than 70° C., or greater than 80° C. In some embodiments, the liquid crystalline domain comprises an ordered lattice at a use temperature, and a disordered morphology when at a temperature greater than the use temperature. As a non-limiting example, material having a use temperature of about 37° C. can have a liquid crystalline domain comprising an ordered lattice at 37° C., and a disordered morphology when warmed to greater than 37° C.

In some embodiments, a polymeric material herein comprises a ratio of crystalline polymeric phases to amorphous polymeric phases (wt/wt) of greater than about 1:10, greater than about 1:9, greater than about 1:8, greater than about 1:7, greater than about 1:6, greater than about 1:5, greater than about 1:4, greater than about 1:3, greater than about 1:2, greater than about 1:1, greater than about 2:1, greater than about 3:1, greater than about 4:1, greater than about 5:1, greater than about 6:1, greater than about 7:1, greater than about 8:1, greater than about 9:1, greater than about 10:1, greater than about 20:1, greater than about 30:1, greater than about 40:1, greater than about 50:1, or greater than about 99:1. In some embodiments, the polymeric material comprises a ratio of the crystallizable polymeric material to the amorphous polymeric material (wt/wt) of at least 1:10, at least 1:9, at least 1:8, at least 1:7, at least 1:6, at least 1:5, at least 1:4, at least 1:3, at least 1:2, at least 1:1, at least 2:1, at least 3:1, at least 4:1, at least 5:1, at least 6:1, at least 7:1, at least 8:1, at least 9:1, at least 10:1, at least 20:1, at least 30:1, at least 40:1, at least 50:1, or at least 99:1. In certain embodiments, the polymeric material comprises a ratio of crystalline polymeric phases to amorphous polymeric phases (wt/wt) of between 1:9 and 99:1, between 1:9 and 9:1, between 1:4 and 4:1, between 1:4 and 1:1, between 3:5 and 1:1, between 1:1 and 5:3, or between 1:1 and 4:1.

In some embodiments, a polymeric material of this disclosure comprises a ratio of crystalline polymeric phases to amorphous polymeric phases (vol/vol) of greater than about 1:10, greater than about 1:9, greater than about 1:8, greater than about 1:7, greater than about 1:6, greater than about 1:5, greater than about 1:4, greater than about 1:3, greater than about 1:2, greater than about 1:1, greater than about 2:1, greater than about 3:1, greater than about 4:1, greater than about 5:1, greater than about 6:1, greater than about 7:1, greater than about 8:1, greater than about 9:1, greater than about 10:1, greater than about 20:1, greater than about 30:1, greater than about 40:1, greater than about 50:1, or greater than about 99:1. In some embodiments, the polymeric material comprises a ratio of crystalline polymeric phases to amorphous polymeric phases (vol/vol) of at least 1:10, at least 1:9, at least 1:8, at least 1:7, at least 1:6, at least 1:5, at least 1:4, at least 1:3, at least 1:2, at least 1:1, at least 2:1, at least 3:1, at least 4:1, at least 5:1, at least 6:1, at least 7:1, at least 8:1, at least 9:1, at least 10:1, at least 20:1, at least 30:1, at least 40:1, at least 50:1, or at least 99:1. In certain embodiments, the polymeric material comprises a ratio of crystalline polymeric phases to amorphous polymeric phases (vol/vol) of between 1:9 and 99:1, between 1:9 and 9:1, between 1:4 and 4:1, between 1:4 and 1:1, between 3:5 and 1:1, between 1:1 and 5:3, or between 1:1 and 4:1.

### Properties of Polymeric Materials

A polymeric material of this disclosure formed from the polymerization of a curable composition disclosed herein can provide advantageous characteristics compared to conventional polymeric materials. In some instances, and as described herein, a polymeric material can contain some percentage of crystallinity, which can impart an increased toughness and high modulus to the polymeric material, while in some circumstances being a 3D printable material. Furthermore, a polymeric material herein can further comprise one or more amorphous phases that can provide increased durability, prevention of crack formation, as well as the prevention of crack propagation. In some instances, a polymeric material can also have low amounts of water uptake, and can be solvent resistant. In some cases, a polymeric material can be characterized by one or more of the properties selected from the group consisting of elongation at break, storage modulus, tensile modulus, flexural stress remaining, glass transition temperature, water uptake, hardness, color, transparency, hydrophobicity, lubricity, surface texture, percent crystallinity, phase composition ratio, phase domain size, and phase domain size and morphology. Further, as described herein, the polymeric materials provided herein can be used for a multitude of applications, including 3D printing, to form materials having favorable properties of both elasticity and stiffness. Specifically, a polymeric material of this disclosure can provide excellent flexural modulus, elastic modulus, elongation at break, or a combination thereof.

In various embodiments, a polymeric material herein can comprise or consist of a high toughness, e.g., through a tough polymer matrix, and the difference (or delta) between the elastic modulus measured at different strain rates (e.g., at 1.7 mm/min and 510 mm/min) can be low, e.g., lower than 80%, 70%, 60%, 50%, 40%, or lower than 30%, which can be an indication for a polymeric phase separation within the material.

In some embodiments, a polymeric material of the present disclosure can have one or more of the following characteristics: (A) a storage modulus greater than or equal to 200 MPa; (B) a flexural stress and/or flexural stress and/or flexural modulus of greater than or equal to 1.5 MPa remaining after 24 hours in a wet environment at 37° C.; (C) an elongation at break greater than or equal to 5% before and after 24 hours in a wet environment at 37° C.; (D) a water uptake of less than 25 wt % when measured after 24 hours in a wet environment at 37° C.; (E) transmission of at least 30% of visible light through the polymeric material after 24 hours in a wet environment at 37° C.; and (F) comprises a plurality of polymeric phases, wherein at least one polymeric phase of the one or more polymeric phases has a Tg of at least 60° C., 80° C., 90° C., 100° C., or at least 110° C. In some instances, a polymeric material herein has at least two, three, four, five, or all characteristics of (A), (B), (C), (D), (E) and (F).

In some instances, the polymeric material can be characterized by a storage modulus of 0.1 MPa to 4000 MPa, a storage modulus of 300 MPa to 3000 MPa, or a storage modulus of 750 MPa to 3000 MPa after 24 hours in a wet environment at 37° C. In some instances, the polymeric material is characterized by a flexural stress and/or flexural modulus of greater than or equal to 5 MPa, greater than or equal to 10 MPa, greater than or equal to 20 MPa, greater than or equal to 30 MPa, greater than or equal to 40 MPa, greater than or equal to 50 MPa, greater than or equal to 60 MPa, greater than or equal to 80 MPa, or greater than or equal to 100 MPa remaining after 24 hours in a wet environment at 37° C.

In some instances, the polymeric material herein can have a flexural stress and/or flexural modulus of 400 MPa or more, 300 MPa or more, 200 MPa or more, 180 MPa or more, 160 MPa or more, 120 MPa or more, 100 MPa or more, 80 MPa or more, 70 MPa or more, 60 MPa or more, after 24 hours in a wet environment at 37° C.

In some instances, the polymeric material can be characterized by an elongation at break greater than 10%, an elongation at break greater than 20%, an elongation at break greater than 30%, an elongation at break of 5% to 250%, an elongation at break of 20% to 250%, or an elongation at break value between 40% and 250% before and after 24 hours in a wet environment at 37° C.

A polymeric material can be characterized by a water uptake of less than 20 wt %, less than 15 wt %, less than 10 wt %, less than 5 wt %, less than 4 wt %, less than 3 wt %, less than 2 wt %, less than 1 wt %, less than 0.5 wt %, less than 0.25 wt %, or less than 0.1 wt % when measured after 24 hours in a wet environment at 37° C. In some cases, a polymeric material can have greater than 50%, 60%, or 70% conversion of double bonds to single bonds compared to the curable composition, as measured by FTIR.

In some embodiments, a polymeric material can have an ultimate tensile strength from 10 MPa to 100 MPa, from 15 MPa to 80 MPa, from 20 MPa to 60 MPa, from 10 MPa to 50 MPa, from 10 MPa to 45 MPa, from 25 MPa to 40 MPa, from 30 MPa to 45 MPa, or from 30 MPa to 40 MPa after 24 hours in a wet environment at 37° C.

In some embodiments, a polymeric material can have a low amount of hydrogen bonding which can facilitate a decreased uptake of water in comparison with conventional polymeric materials having greater amounts of hydrogen bonding. Thus, in some instances, a polymeric material herein can comprise less than about 10 wt %, less than about 9 wt %, less than about 8 wt %, less than about 7 wt %, less than about 6 wt %, less than about 5 wt %, less than about 4 wt %, less than about 3 wt %, less than about 2 wt %, less than about 1 wt %, or less than about 0.5 wt % water when fully saturated fat use temperature (e.g., about 20° C., 25° C., 30° C., or 35° C.). In some instances, the use temperature can include the temperature of a human mouth (e.g., approximately 35-40° C.). The use temperature can be a temperature selected from −100-250° C., 0-90° C., 0-80° C., 0-70° C., 0-60° C., 0-50° C., 0-40° C., 0-30° C., 0-20° C., 0-10° C., 20-90° C., 20-80° C., 20-70° C., 20-60° C., 20-50° C., 20-40° C., 20-30° C., or below 0° C.

In some embodiments, a polymeric material herein comprises at least one crystalline phase and at least one amorphous phase, wherein the at least one crystalline phase contains rigid segments, for example, liquid crystalline segments, and the at least one amorphous phase contains flexible segments of polymerizable compounds of the present disclosure. In some instance, a combination of these two types of phases or domains can create a polymeric material that has a high modulus phase (e.g., the crystalline polymeric material can provide a high modulus) and a low modulus phase (e.g., provided by the presence of the amorphous polymeric material). By having these two phases, the polymeric material can have a high modulus and a high elongation, as well as high flexural stress remaining following stress relaxation.

In various instances, the one or more amorphous phases of the polymeric material can have a glass transition temperature of at least about 30° C., 40° C., 50° C., 60° C., 70° C., 80° C., 90° C., 100° C., or at least about 110° C. In such cases, at least one amorphous phase of the one or more amorphous phases having a glass transition temperature of at least about 50° C. comprises, integrated in its polymeric structure, flexible segments of a polymerizable compounds of the present disclosure.

In some embodiments, a polymeric material herein can comprise crystalline and/or amorphous phases having a smaller size (e.g., less than about 5 μm). Smaller polymeric phases in a polymeric material can facilitate light passage and provide a polymeric material that appears clear. In contrast, larger polymeric phases (e.g., those larger than about 1 μm) can scatter light, for example when the refractive index of the polymer crystal is different from the refractive index of the amorphous phase adjacent to the polymer crystal (e.g., the amorphous material). In some cases, at least 40%, 50%, 60%, or 70% of visible light passes through the polymeric material after 24 hours in a wet environment at 37° C.

Thus, in some cases, it may be advantageous to have a polymeric material that comprises small polymeric phases such as crystalline or amorphous phases, e.g., as measured by the longest length of the phases. In some embodiments, such polymeric material comprises an average polymeric phase size that is less than 5 μm. In some cases, the maximum polymeric phase size of the polymeric materials can be about 5 μm. In some embodiments, at least about 50%, at least about 60%, at least about 70%, at least about 80%, at least about 90%, at least about 95%, or at least about 99% of the polymeric phases of the polymeric material have a size of less than about 5 μm. In yet other embodiments, a polymeric material comprises an average polymeric phase size that is less than about 1 μm. In some embodiments, the maximum polymer polymeric phase size of the cured polymeric materials is 1 μm. In some embodiments, at least about 50%, at least about 60%, at least about 70%, at least about 80%, at least about 90%, at least about 95%, or at least about 99% of the polymeric phases of the polymeric material have a size less than about 1 μm. In yet other embodiments, the polymeric material comprises an average polymeric phase size that is less than about 500 nm. In some embodiments, the maximum polymeric phase size of the cured polymeric materials is about 500 nm. In some embodiments, at least about 50%, at least about 60%, at least about 70%, at least about 80%, at least about 90%, at least about 95%, or at least about 99% of the polymeric phases of the polymeric material have a size less than 500 nm.

In some embodiments, the size of at least one or more of the polymeric phases (e.g., crystalline phases and amorphous phases) of a polymeric material can be controlled. Non-limiting examples of ways in which the size of the polymeric phases can be controlled includes: rapidly cooling the cured polymeric material, annealing the cured polymeric material at an elevated temperature (i.e., above room temperature), annealing the cured polymeric material at a temperature below room temperature, controlling the rate of polymerization, controlling the intensity of light during the curing step using light, controlling and/or adjusting polymerization temperature, exposing the cured polymeric material to sonic vibrations, and/or controlling the presence and amounts of impurities, and in particular for crystalline phases, adding crystallization-inducing chemicals or particles (e.g., crystallization seeds).

In some embodiments, the refractive index of the one or more crystalline phases and/or one or more amorphous phases of a polymeric material herein can be controlled. A reduction in difference of refractive index between different phases (e.g., reduction in the difference of refractive index between the crystalline polymer and the amorphous polymer) can increase clarity of the cured polymeric material, providing a clear or nearly clear material. Light scatter can be decreased by minimizing polymer crystal size, as well as by reducing the difference of refractive index across an interface between an amorphous polymeric phase and a crystalline phase. In some embodiments, the difference of refractive index between a given polymeric phase and a neighboring phase (e.g., crystalline and a neighboring amorphous phase) can be less than about 0.1, less than about 0.01, or less than about 0.001.

Further provided herein are polymeric films comprising a polymeric material of the present disclosure. In some cases, such polymeric film can have a thickness of at least about 50 μm, 100 μm, 250 μm, 500 μm, 1 mm, 2 mm and not more than 3 mm.

### Polymeric Materials in Medical Devices

The present disclosure provides devices that comprise a polymeric material of the present disclosure. As described herein, such polymeric material can comprise, incorporated in its polymeric structure, one or more polymerizable components of this disclosure. In various cases, the device can be a medical device. The medical device can be an orthodontic appliance. The orthodontic appliance can be a dental aligner, a dental expander or a dental spacer.

### Methods of Use

The present disclosure provides methods of using compositions comprising polymerizable compounds herein, as well as methods for using the compositions in devices such as orthodontic devices.

### Methods of Forming Polymeric Materials

In some embodiments, the present disclosure provides methods of producing the polymeric materials from the curable compositions described herein. In various embodiments, the method comprises the steps of: (i) providing a curable composition of the present disclosure; (ii) exposing the curable composition to a light source; and curing the curable composition, thereby forming the polymeric material.

In some embodiments, the photo-curing comprises a single curing step. In some embodiments, the photo-curing comprises a plurality of curing steps. In yet other embodiments, the photo-curing comprises at least one curing step which exposes the curable composition to light. Exposing the curable composition to light can initiate and/or facilitate photo-polymerization. In some instances, a photoinitiator can be used as part of the curable composition to accelerate and/or initiate photo-polymerization. In some embodiments, the curable composition is exposed to UV (ultraviolet) light, visible light, IR (infrared) light, or any combination thereof. In some embodiments, the cured polymeric material is formed from the curable composition using at least one step comprising exposure to a light source, wherein the light source comprises UV light, visible light, and/or IR light. In some embodiments, the light source comprises a wavelength from 10 nm to 200 nm, from 200 nm to 350 nm, from 350 nm to 450 nm, from 450 nm to 550 nm, from 550 nm to 650 nm, from 650 nm to 750 nm, from 750 nm to 850 nm, from 850 nm to 1000 nm, or from 1000 nm to 1500 nm.

In some embodiments, a method of forming a polymeric material from a curable composition described herein can further comprise inducing phase separation in the forming the polymeric material (i.e., during photo-curing), wherein such phase separation can be polymerization-induced. The polymerization-induced phase separation can comprise generating one or more polymeric phases in the polymeric material during photo-curing. In some cases, at least one polymeric phase of the one or more polymeric phases is an amorphous polymeric phase. Such at least one amorphous polymeric phase can have a glass transition temperature (Tg) of at least about 40° C., 50° C., 60° C., 80° C., 90° C., 100° C., 110° C. or at least about 120° C. In some cases, at least 25%, 50%, or 75% of polymeric phases generated during photo-curing have a glass transition temperature (Tg) of at least about 40° C., 50° C., 60° C., 80° C., 90° C., 100° C., 110° C. or at least about 120° C. In various cases, at least one polymeric phase of the one or more polymeric phases generated during photo-curing comprises a crystalline polymeric material. Hence, in some cases, at least one polymeric phase of the one or more polymeric phases is a crystalline polymeric phase. The crystalline polymeric material (e.g., as part of a crystalline phase) can have a melting point of at least about 40° C., 50° C., 60° C., 80° C., 90° C., 100° C., 110° C. or at least about 120° C.

In some embodiments, a method of forming a polymeric material from a photo-polymerizable composition described herein can further comprise initiating and/or enhancing formation of crystalline phases in the forming polymeric material. In certain embodiments, the curable composition consists substantially of an amorphous phase prior to curing, and following the curing into the cured polymeric material, there exists a percentage of crystalline domains comprising crystals. As a non-limiting example, the curable composition can be a solid at room temperature, then heated into a liquid state, then cured (e.g., the curable composition can be irradiated with light, causing polymerization to occur) in which the material becomes a solid. The curing step may optionally comprise more than one step; for example, the cured material from the previous sentence can be heated (e.g., placed in an oven), and a second polymerization may occur which further polymerizes material. In some embodiments, the polymeric material is crystal free immediately following and/or shortly after the curing step. In some embodiments, the curing of the curable composition is at an elevated temperature, and as the cured polymeric material cools to room temperature (i.e., 25° C.), the cooling can trigger the formation and/or growth of crystals. In some embodiments, the crystallization may occur at some time after curing, such as 5 minutes after, 30 minutes after, 1 hour after, or longer. Also, in some embodiments, the crystallization does not occur until the material is annealed at a temperature that facilitates the crystallization process. Delayed crystallization (for 3D printing that involves layers) is particularly advantageous as it allows for isotropic shrinkage to occur if the crystallization across the whole printed part occurs at all at one time, preventing shrinkage stress induced warping of the part.

In certain embodiments, the triggering of crystallization comprises cooling the cured material, adding seeding particles to the resin, providing a force to the cured material, providing an electrical charge to the resin, or any combination thereof. In some cases, polymer crystals can yield upon application of a strain (e.g., a physical strain, such as twisting or stretching a material). The yielding may include unraveling, unwinding, disentangling, dislocation, coarse slips, and/or fine slips in the crystallized polymer. In some embodiments, the methods disclosed herein further comprise the step of growing polymer crystals. As described further herein, polymer crystals comprise the crystallizable polymeric material.

Thus, in various embodiments, a method of forming a polymeric material from a curable composition described herein can comprise inducing phase separation in the forming polymeric material (i.e., during photo-curing), wherein such phase separation can yield polymeric materials that comprise one or more amorphous phases, one or more crystalline phases, or both one or more amorphous phases and one or more crystalline phases.

As described herein, a polymeric material produced by the methods provided herein can be characterized by one or more of: (i) a storage modulus greater than or equal to 200 MPa; (ii) a flexural stress and/or flexural modulus of greater than or equal to 1.5 MPa remaining after 24 hours in a wet environment at 37° C.; (iii) an elongation at break greater than or equal to 5% before and after 24 hours in a wet environment at 37° C.; (iv) a water uptake of less than 25 wt % when measured after 24 hours in a wet environment at 37° C.; and (v) transmission of at least 30% of visible light through the polymeric material after 24 hours in a wet environment at 37° C. In various cases, such polymeric material can be characterized by at least 2, 3, 4, or all of these properties.

### Fabrication and Use of Orthodontic Appliances

Provided herein are methods for using the polymerizable compounds of structures (I), (II) or (III) and curable (i.e., polymerizable) compositions comprising such polymerizable compounds, as well as polymeric materials produced from such compositions for the fabrication of a medical device, such as an orthodontic appliance (e.g., a dental aligner, a dental expander or a dental spacer).

Thus, in some embodiments, a method herein further comprises the step of fabricating a device or an object using an additive manufacturing device, wherein the additive manufacturing device facilitates the curing. In some embodiments, the curing of a polymerizable composition produces the cured polymeric material. In certain embodiments, a polymerizable composition is cured using an additive manufacturing device to produce the cured polymeric material. In some embodiments, the method further comprises the step of cleaning the cured polymeric material. In certain embodiments, the cleaning of the cured polymeric material includes washing and/or rinsing the cured polymeric material with a solvent, which can remove uncured resin and undesired impurities from the cured polymeric material.

In some embodiments, a polymerizable composition herein can be curable and have melting points <100° C. in order to be liquid and, thus, processable at the temperatures usually employed in currently available additive manufacturing techniques. As described herein, the polymerizable compounds of structure (I), (II) or (III) of the present disclosure that are used as components in the curable compositions can have a low viscosity at an elevated temperature. Such low viscosity of the polymerizable compounds described herein can be particularly advantageous for use of such component in the curable (e.g., photocurable) compositions and additive manufacturing where elevated temperatures (e.g., 60° C., 80° C., 90° C., or higher) may be used. In various instances, various polymerizable compounds can have a vapor pressure of at most about 12 Pa at 60° C., or lower, as further described herein.

In some embodiments, a curable composition herein can comprise at least one photo-polymerization initiator (i.e., a photoinitiator) and may be heated to a predefined elevated process temperature ranging from about 50° C. to about 120° C., such as from about 90° C. to about 120° C., before becoming irradiated with light of a suitable wavelength to be absorbed by the photoinitiator, thereby causing activation of the photoinitiator to induce polymerization of the curable composition to obtain a cured polymeric material, which an optionally be cross-linked. In some embodiments, the curable composition can comprise at least one multivalent polymerizable monomer that can provide a cross-linked polymer.

In some embodiments, the methods disclosed herein for forming a polymeric material are part of a high temperature lithography-based photo-polymerization process, wherein a curable composition (e.g., a photo-curable curable composition) that can comprise at least one photo-polymerization initiator is heated to an elevated process temperature (e.g., from about 50° C. to about 120° C., such as from about 90° C. to about 120° C.). Thus, a method for forming a polymeric material according to the present disclosure can offer the possibility of quickly and facilely producing devices, such as orthodontic appliances, by additive manufacturing such as 3D printing using curable compositions as disclosed herein. In various embodiments, such curable composition comprising one or more polymerizable compounds of structure (I), (II) or (III) of the present disclosure.

Photo-polymerization can occur when a curable composition herein is exposed to radiation (e.g., UV or visible light) of a wavelength sufficient to initiate polymerization. The wavelengths of radiation useful to initiate polymerization may depend on the photoinitiator used. “Light” as used herein includes any wavelength and power capable of initiating polymerization. Some wavelengths of light include ultraviolet (UV) or visible. UV light sources include UVA (wavelength about 400 nanometers (nm) to about 320 nm), UVB (about 320 nm to about 290 nm) or UVC (about 290 nm to about 100 nm). Any suitable source may be used, including laser sources. The source may be broadband or narrowband, or a combination thereof. The light source may provide continuous or pulsed light during the process. Both the length of time the system is exposed to UV light and the intensity of the UV light can be varied to determine the ideal reaction conditions.

In some embodiments, the methods disclosed herein include the use of additive manufacturing to produce a device comprising the cured polymeric material. Such device can be an orthodontic appliance. The orthodontic appliance can be a dental aligner, a dental expander or a dental spacer. In certain embodiments, the methods disclosed herein use additive manufacturing to produce a device comprising, consisting essentially of, or consisting of the cured polymeric material. Additive manufacturing includes a variety of technologies which fabricate three-dimensional objects directly from digital models through an additive process. In some embodiments, successive layers of material are deposited and “cured in place”. A variety of techniques are known to the art for additive manufacturing, including selective laser sintering (SLS), fused deposition modeling (FDM) and jetting or extrusion. In many embodiments, selective laser sintering involves using a laser beam to selectively melt and fuse a layer of powdered material according to a desired cross-sectional shape in order to build up the object geometry. In many embodiments, fused deposition modeling involves melting and selectively depositing a thin filament of thermoplastic polymer in a layer-by-layer manner in order to form an object. In yet another example, 3D printing can be used to fabricate an orthodontic appliance herein. In many embodiments, 3D printing involves jetting or extruding one or more materials (e.g., the crystallizable resins disclosed herein) onto a build surface in order to form successive layers of the object geometry. In some embodiments, a curable composition described herein can be used in inkjet or coating applications. Cured polymeric materials may also be fabricated by “vat” processes in which light is used to selectively cure a vat or reservoir of the curable resin. Each layer of curable resin may be selectively exposed to light in a single exposure or by scanning a beam of light across the layer. Specific techniques that can be used herein can include stereolithography (SLA), Digital Light Processing (DLP) and two photon-induced photo-polymerization (TPIP).

In some embodiments, the methods disclosed herein use continuous direct fabrication to produce a device comprising the cured polymeric material. Such device can be an orthodontic appliance as described herein. In certain embodiments, the methods disclosed herein can comprise the use of continuous direct fabrication to produce a device (e.g., an orthodontic appliance) comprising, consisting essentially of, or consisting of the cured polymeric material. A non-limiting exemplary direct fabrication process can achieve continuous build-up of an object geometry by continuous movement of a build platform (e.g., along the vertical or Z-direction) during an irradiation phase, such that the hardening depth of the irradiated photo-polymer (e.g., an irradiated curable composition, hardening during the formation of a cured polymeric material) is controlled by the movement speed. Accordingly, continuous polymerization of material (e.g., polymerization of a curable composition into a cured polymeric material) on the build surface can be achieved. Such methods are described in U.S. Pat. No. 7,892,474, the disclosure of which is incorporated herein by reference in its entirety. In yet another example, a continuous direct fabrication method utilizes a “heliolithography” approach in which a liquid resin (e.g., a curable composition) is cured with focused radiation while the build platform is continuously rotated and raised. Accordingly, the object geometry can be continuously built up along a spiral build path. Such methods are described in U.S. Patent Publication No. 2014/0265034, the disclosure of which is incorporated herein by reference in its entirety. Continuous liquid interface production of 3D objects has also been reported (J. Tumbleston et al., Science, 2015, 347 (6228), pp 1349-1352), which reference is hereby incorporated by reference in its entirety for description of the process. Another example of continuous direct fabrication method can involve extruding a material composed of a curable liquid material or resin surrounding a solid strand. The material can be extruded along a continuous three-dimensional path in order to form the object. Such methods are described in U.S. Patent Publication No. 2014/0061974, the disclosure of which is incorporated herein by reference in its entirety.

In some embodiments, the methods disclosed herein can comprise the use of high temperature lithography to produce a device comprising the cured polymeric material. Such device can be an orthodontic appliance as described herein. In certain embodiments, the methods disclosed herein use high temperature lithography to produce a device comprising, consisting essentially of, or consisting of the cured polymeric material. “High temperature lithography,” as used herein, may refer to any lithography-based photo-polymerization processes that involve heating photo-polymerizable material(s) (e.g., a curable composition disclosed herein). The heating may lower the viscosity of the curable composition before and/or during curing. Non-limiting examples of high-temperature lithography processes include those processes described in WO 2015/075094, WO 2016/078838 and WO 2018/032022. In some implementations, high-temperature lithography may involve applying heat to material to temperatures from about 50° C. to about 120° C., such as from about 90° C. to about 120° C., from about 100° C. to about 120° C., from about 105° C. to about 115° C., from about 108° C. to about 110° C., etc. The material may be heated to temperatures greater than about 120° C. It is noted other temperature ranges may be used without departing from the scope and substance of the inventive concepts described herein.

Since, in some cases, the polymerizable compounds of the present disclosure can, as part of a curable composition, become co-polymerized in the polymerization process of a method according to the present disclosure, the result can be an optionally cross-linked polymer comprising moieties of one or more species of the polymerizable compounds as repeating units. In some cases, such polymer is a cross-linked polymer which, typically, can be suitable and useful for applications in orthodontic appliances.

In further embodiments, a method herein can comprise polymerizing a curable composition which comprises at least one multivalent monomer, which, upon polymerization, can furnish a cross-linked polymer which can comprise moieties originating from the polymerizable compounds of the present disclosure as repeating units. In order to obtain cross-linked polymers which can be particularly suitable as orthodontic appliances, the at least one polymerizable species used in the method according to the present disclosure can be selected with regard to several thermomechanical properties of the resulting polymers. In some instances, a curable resin of the present disclosure can comprise one or more species of multivalent polymerizable monomers.

### Orthodontic Appliances and Uses Thereof

The polymerizable compounds/monomers of the present disclosure can be used as components for viscous or highly viscous curable composition s and can result in polymeric materials that can have favorable thermomechanical properties as described herein (e.g., stiffness, flexural stress remaining, etc.) for use in orthodontic appliances, for example, for moving one or more teeth of a patient.

As described herein, the present disclosure provides a method of repositioning a patient's teeth, the method comprising: (i) generating a treatment plan for the patient, the plan comprising a plurality of intermediate tooth arrangements for moving teeth along a treatment path from an initial tooth arrangement toward a final tooth arrangement; (ii) producing an orthodontic appliance comprising a polymeric material described herein, e.g., a polymeric material that comprises a polymerizable compound of the present disclosure; and moving on-track, with the orthodontic appliance, at least one of the patient's teeth toward an intermediate tooth arrangement or the final tooth arrangement. Such orthodontic appliance can be produced using processes that include 3D printing, as further described herein. The method of repositioning a patient's teeth can further comprise tracking progression of the patient's teeth along the treatment path after administration of the orthodontic appliance to the patient, the tracking comprising comparing a current arrangement of the patient's teeth to a planned arrangement of the patient's teeth. In such instances, greater than 60% of the patient's teeth can be on track with the treatment plan after 2 weeks of treatment. In some instances, the orthodontic appliance has a retained repositioning force to the at least one of the patient's teeth after 2 days that is at least 10%, at least 20%, at least 30%, at least 40%, at least 50%, at least 60%, or at least 70% of repositioning force initially provided to the at least one of the patient's teeth.

As used herein, the terms “rigidity” and “stiffness” can be used interchangeably, as are the corresponding terms “rigid” and “stiff.” As used herein a “plurality of teeth” encompasses two or more teeth.

In many embodiments, one or more posterior teeth comprises one or more of a molar, a premolar or a canine, and one or more anterior teeth comprising one or more of a central incisor, a lateral incisor, a cuspid, a first bicuspid or a second bicuspid.

In some embodiments, the compositions and methods described herein can be used to couple groups of one or more teeth to each other. The groups of one or more teeth may comprise a first group of one or more anterior teeth and a second group of one or more posterior teeth. The first group of teeth can be coupled to the second group of teeth with the polymeric shell appliances as disclosed herein.

The embodiments disclosed herein are well suited for moving one or more teeth of the first group of one or more teeth or moving one or more of the second group of one or more teeth, and combinations thereof.

The embodiments disclosed herein are well suited for combination with one or more known commercially available tooth moving components such as attachments and polymeric shell appliances. In many embodiments, the appliance and one or more attachments are configured to move one or more teeth along a tooth movement vector comprising six degrees of freedom, in which three degrees of freedom are rotational and three degrees of freedom are translation.

The present disclosure provides orthodontic systems and related methods for designing and providing improved or more effective tooth moving systems for eliciting a desired tooth movement and/or repositioning teeth into a desired arrangement.

Although reference is made to an appliance comprising a polymeric shell appliance, the embodiments disclosed herein are well suited for use with many appliances that receive teeth, for example appliances without one or more of polymers or shells. The appliance can be fabricated with one or more of many materials such as metal, glass, reinforced fibers, carbon fiber, composites, reinforced composites, aluminum, biological materials, and combinations thereof, for example. In some cases, the reinforced composites can comprise a polymer matrix reinforced with ceramic or metallic particles, for example. The appliance can be shaped in many ways, such as with thermoforming or direct fabrication as described herein, for example. Alternatively, or in combination, the appliance can be fabricated with machining such as an appliance fabricated from a block of material with computer numeric control machining. In some cases, the appliance is fabricated using a polymerizable compound according to the present disclosure, for example, using the polymerizable compounds as reactive diluents or as toughness modifiers for curable resins.

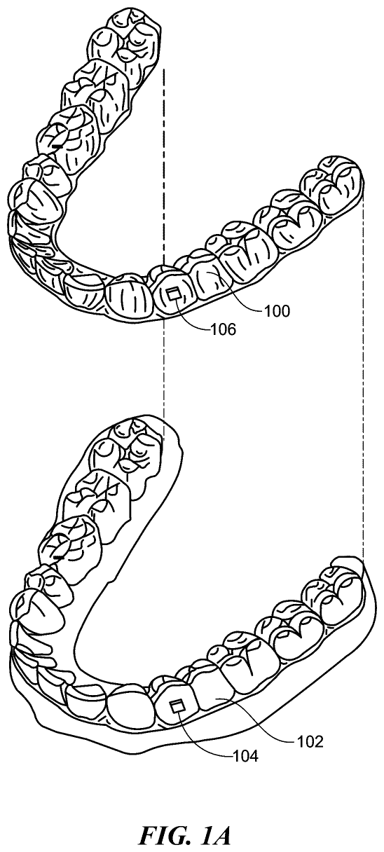

Turning now to the drawings, in which like numbers designate like elements in the various figures, FIG. 1A illustrates an exemplary tooth repositioning appliance or aligner 100 that can be worn by a patient in order to achieve an incremental repositioning of individual teeth 102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using rapid prototyping fabrication techniques, from a digital model of an appliance. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some, most, or even all of the teeth will be repositioned at some point during treatment. Teeth that are moved can also serve as a base or anchor for holding the appliance as it is worn by the patient. Typically, no wires or other means will be provided for holding an appliance in place over the teeth. In some cases, however, it may be desirable or necessary to provide individual attachments or other anchoring elements 104 on teeth 102 with corresponding receptacles or apertures 106 in the appliance 100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 1B illustrates a tooth repositioning system 110 including a plurality of appliances 112, 114, 116. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 110 can include a first appliance 112 corresponding to an initial tooth arrangement, one or more intermediate appliances 114 corresponding to one or more intermediate arrangements, and a final appliance 116 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

FIG. 1C illustrates a method 150 of orthodontic treatment using a plurality of appliances, in accordance with embodiments. The method 150 can be practiced using any of the appliances or appliance sets described herein. In step 160, a first orthodontic appliance is applied to a patient's teeth in order to reposition the teeth from a first tooth arrangement to a second tooth arrangement. In step 170, a second orthodontic appliance is applied to the patient's teeth in order to reposition the teeth from the second tooth arrangement to a third tooth arrangement. The method 150 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

The various embodiments of the orthodontic appliances presented herein can be fabricated in a wide variety of ways. In some embodiments, the orthodontic appliances herein (or portions thereof) can be produced using direct fabrication, such as additive manufacturing techniques (also referred to herein as “3D printing”) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photo-polymerization (e.g., stereolithography), in which an object is constructed layer by layer from a vat of liquid photo-polymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive resin (e.g., a photo-polymer) according to a desired cross-sectional shape using light (e.g., ultraviolet light). The object geometry can be built up in a layer-by-layer fashion by sequentially polymerizing a plurality of object cross-sections. As another example, the appliances herein can be directly fabricated using selective laser sintering. In some embodiments, selective laser sintering involves using a laser beam to selectively melt and fuse a layer of powdered material according to a desired cross-sectional shape in order to build up the object geometry. As yet another example, the appliances herein can be directly fabricated by fused deposition modeling. In some embodiments, fused deposition modeling involves melting and selectively depositing a thin filament of thermoplastic polymer in a layer-by-layer manner in order to form an object. In yet another example, material jetting can be used to directly fabricate the appliances herein. In some embodiments, material jetting involves jetting or extruding one or more materials onto a build surface in order to form successive layers of the object geometry.

Alternatively, or in combination, some embodiments of the appliances herein (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

In some embodiments, the direct fabrication methods provided herein build up the object geometry in a layer-by-layer fashion, with successive layers being formed in discrete build steps. Alternatively, or in combination, direct fabrication methods that allow for continuous build-up of an object geometry can be used, referred to herein as “continuous direct fabrication.” Diverse types of continuous direct fabrication methods can be used. As an example, in some embodiments, the appliances herein are fabricated using “continuous liquid interphase printing,” in which an object is continuously built up from a reservoir of photo-polymerizable resin by forming a gradient of partially cured resin between the building surface of the object and a polymerization-inhibited “dead zone.” In some embodiments, a semi-permeable membrane is used to control transport of a photo-polymerization inhibitor (e.g., oxygen) into the dead zone in order to form the polymerization gradient. Continuous liquid interphase printing can achieve fabrication speeds about 25 times to about 100 times faster than other direct fabrication methods, and speeds about 1000 times faster can be achieved with the incorporation of cooling systems. Continuous liquid interphase printing is described in U.S. Patent Publication Nos. 2015/0097315, 2015/0097316, and 2015/0102532, the disclosures of each of which are incorporated herein by reference in their entirety.

As another example, a continuous direct fabrication method can achieve continuous build-up of an object geometry by continuous movement of the build platform (e.g., along the vertical or Z-direction) during the irradiation phase, such that the hardening depth of the irradiated photo-polymer is controlled by the movement speed. Accordingly, continuous polymerization of material on the build surface can be achieved. Such methods are described in U.S. Pat. No. 7,892,474, the disclosure of which is incorporated herein by reference in its entirety.

In another example, a continuous direct fabrication method can involve extruding a composite material composed of a curable liquid material surrounding a solid strand. The composite material can be extruded along a continuous three-dimensional path in order to form the object. Such methods are described in U.S. Patent Publication No. 2014/0061974, the disclosure of which is incorporated herein by reference in its entirety.

In yet another example, a continuous direct fabrication method utilizes a “heliolithography” approach in which the liquid photo-polymer is cured with focused radiation while the build platform is continuously rotated and raised. Accordingly, the object geometry can be continuously built up along a spiral build path. Such methods are described in U.S. Patent Publication No. 2014/0265034, the disclosure of which is incorporated herein by reference in its entirety.

The direct fabrication approaches provided herein are compatible with a wide variety of materials, including but not limited to one or more of the following: a polyester, a co-polyester, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermoplastic elastomer (TPE), a thermoplastic vulcanizate (TPV) elastomer, a polyurethane elastomer, a block copolymer elastomer, a polyolefin blend elastomer, a thermoplastic co-polyester elastomer, a thermoplastic polyamide elastomer, a thermoset material, or combinations thereof. The materials used for direct fabrication can be provided in an uncured form (e.g., as a liquid, resin, powder, etc.) and can be cured (e.g., by photo-polymerization, light curing, gas curing, laser curing, cross-linking, etc.) in order to form an orthodontic appliance or a portion thereof. The properties of the material before curing may differ from the properties of the material after curing. Once cured, the materials herein can exhibit sufficient strength, stiffness, durability, biocompatibility, etc. for use in an orthodontic appliance. The post-curing properties of the materials used can be selected according to the desired properties for the corresponding portions of the appliance.

In some embodiments, relatively rigid portions of the orthodontic appliance can be formed via direct fabrication using one or more of the following materials: a polyester, a co-polyester, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, and/or a polytrimethylene terephthalate.

In some embodiments, relatively elastic portions of the orthodontic appliance can be formed via direct fabrication using one or more of the following materials: a styrenic block copolymer (SBC), a silicone rubber, an elastomeric alloy, a thermoplastic elastomer (TPE), a thermoplastic vulcanizate (TPV) elastomer, a polyurethane elastomer, a block copolymer elastomer, a polyolefin blend elastomer, a thermoplastic co-polyester elastomer, and/or a thermoplastic polyamide elastomer.

Machine parameters can include curing parameters. For digital light processing (DLP)-based curing systems, curing parameters can include power, curing time, and/or grayscale of the full image. For laser-based curing systems, curing parameters can include power, speed, beam size, beam shape and/or power distribution of the beam. For printing systems, curing parameters can include material drop size, viscosity, and/or curing power. These machine parameters can be monitored and adjusted on a regular basis (e.g., some parameters at every 1-x layers and some parameters after each build) as part of the process control on the fabrication machine. Process control can be achieved by including a sensor on the machine that measures power and other beam parameters every layer or every few seconds and automatically adjusts them with a feedback loop. For DLP machines, gray scale can be measured and calibrated before, during, and/or at the end of each build, and/or at predetermined time intervals (e.g., every nth build, once per hour, once per day, once per week, etc.), depending on the stability of the system. In addition, material properties and/or photo-characteristics can be provided to the fabrication machine, and a machine process control module can use these parameters to adjust machine parameters (e.g., power, time, gray scale, etc.) to compensate for variability in material properties. By implementing process controls for the fabrication machine, reduced variability in appliance accuracy and residual stress can be achieved.

Optionally, the direct fabrication methods described herein allow for fabrication of an appliance including multiple materials, referred to herein as “multi-material direct fabrication.” In some embodiments, a multi-material direct fabrication method involves concurrently forming an object from multiple materials in a single manufacturing step. For instance, a multi-tip extrusion apparatus can be used to selectively dispense multiple types of materials from distinct material supply sources in order to fabricate an object from a plurality of unconventional materials. Such methods are described in U.S. Pat. No. 6,749,414, the disclosure of which is incorporated herein by reference in its entirety. Alternatively, or in combination, a multi-material direct fabrication method can involve forming an object from multiple materials in a plurality of sequential manufacturing steps. For instance, a first portion of the object can be formed from a first material in accordance with any of the direct fabrication methods herein, then a second portion of the object can be formed from a second material in accordance with methods herein, and so on, until the entirety of the object has been formed.

Direct fabrication can provide various advantages compared to other manufacturing approaches. For instance, in contrast to indirect fabrication, direct fabrication permits production of an orthodontic appliance without utilizing any molds or templates for shaping the appliance, thus reducing the number of manufacturing steps involved and improving the resolution and accuracy of the final appliance geometry. Additionally, direct fabrication permits precise control over the three-dimensional geometry of the appliance, such as the appliance thickness. Complex structures and/or auxiliary components can be formed integrally as a single piece with the appliance shell in a single manufacturing step, rather than being added to the shell in a separate manufacturing step. In some embodiments, direct fabrication is used to produce appliance geometries that would be difficult to create using alternative manufacturing techniques, such as appliances with very small or fine features, complex geometric shapes, undercuts, interproximal structures, shells with variable thicknesses, and/or internal structures (e.g., for improving strength with reduced weight and material usage). For example, in some embodiments, the direct fabrication approaches herein permit fabrication of an orthodontic appliance with feature sizes of less than or equal to about 5 μm, or within a range from about 5 μm to about 50 μm, or within a range from about 20 μm to about 50 μm.

The direct fabrication techniques described herein can be used to produce appliances with substantially isotropic material properties, e.g., substantially the same or similar strengths along all directions. In some embodiments, the direct fabrication approaches herein permit production of an orthodontic appliance with a strength that varies by no more than about 25%, about 20%, about 15%, about 10%, about 5%, about 1%, or about 0.5% along all directions. Additionally, the direct fabrication approaches herein can be used to produce orthodontic appliances at a faster speed compared to other manufacturing techniques. In some embodiments, the direct fabrication approaches herein allow for production of an orthodontic appliance in a time interval less than or equal to about 1 hour, about 30 minutes, about 25 minutes, about 20 minutes, about 15 minutes, about 10 minutes, about 5 minutes, about 4 minutes, about 3 minutes, about 2 minutes, about 1 minutes, or about 30 seconds. Such manufacturing speeds allow for rapid “chair-side” production of customized appliances, e.g., during a routine appointment or checkup.

In some embodiments, the direct fabrication methods described herein implement process controls for various machine parameters of a direct fabrication system or device in order to ensure that the resultant appliances are fabricated with a high degree of precision. Such precision can be beneficial for ensuring accurate delivery of a desired force system to the teeth in order to effectively elicit tooth movements. Process controls can be implemented to account for process variability arising from multiple sources, such as the material properties, machine parameters, environmental variables, and/or post-processing parameters.

Material properties may vary depending on the properties of raw materials, purity of raw materials, and/or process variables during mixing of the raw materials. In many embodiments, resins or other materials for direct fabrication should be manufactured with tight process control to ensure little variability in photo-characteristics, material properties (e.g., viscosity, surface tension), physical properties (e.g., modulus, strength, elongation) and/or thermal properties (e.g., glass transition temperature, heat deflection temperature). Process control for a material manufacturing process can be achieved with screening of raw materials for physical properties and/or control of temperature, humidity, and/or other process parameters during the mixing process. By implementing process controls for the material manufacturing procedure, reduced variability of process parameters and more uniform material properties for each batch of material can be achieved. Residual variability in material properties can be compensated with process control on the machine, as discussed further herein.

Machine parameters can include curing parameters. For digital light processing (DLP)-based curing systems, curing parameters can include power, curing time, and/or grayscale of the full image. For laser-based curing systems, curing parameters can include power, speed, beam size, beam shape and/or power distribution of the beam. For printing systems, curing parameters can include material drop size, viscosity, and/or curing power. These machine parameters can be monitored and adjusted on a regular basis (e.g., some parameters at every 1-x layers and some parameters after each build) as part of the process control on the fabrication machine. Process control can be achieved by including a sensor on the machine that measures power and other beam parameters every layer or every few seconds and automatically adjusts them with a feedback loop. For DLP machines, gray scale can be measured and calibrated at the end of each build. In addition, material properties and/or photo-characteristics can be provided to the fabrication machine, and a machine process control module can use these parameters to adjust machine parameters (e.g., power, time, gray scale, etc.) to compensate for variability in material properties. By implementing process controls for the fabrication machine, reduced variability in appliance accuracy and residual stress can be achieved.

In many embodiments, environmental variables (e.g., temperature, humidity, Sunlight or exposure to other energy/curing source) are maintained in a tight range to reduce variability in appliance thickness and/or other properties. Optionally, machine parameters can be adjusted to compensate for environmental variables.

In many embodiments, post-processing of appliances includes cleaning, post-curing, and/or support removal processes. Relevant post-processing parameters can include purity of cleaning agent, cleaning pressure and/or temperature, cleaning time, post-curing energy and/or time, and/or consistency of support removal process. These parameters can be measured and adjusted as part of a process control scheme. In addition, appliance physical properties can be varied by modifying the post-processing parameters. Adjusting post-processing machine parameters can provide another way to compensate for variability in material properties and/or machine properties.

The configuration of the orthodontic appliances herein can be determined according to a treatment plan for a patient, e.g., a treatment plan involving successive administration of a plurality of appliances for incrementally repositioning teeth. Computer-based treatment planning and/or appliance manufacturing methods can be used in order to facilitate the design and fabrication of appliances. For instance, one or more of the appliance components described herein can be digitally designed and fabricated with the aid of computer-controlled manufacturing devices (e.g., computer numerical control (CNC) milling, computer-controlled rapid prototyping such as 3D printing, etc.). The computer-based methods presented herein can improve the accuracy, flexibility, and convenience of appliance fabrication.

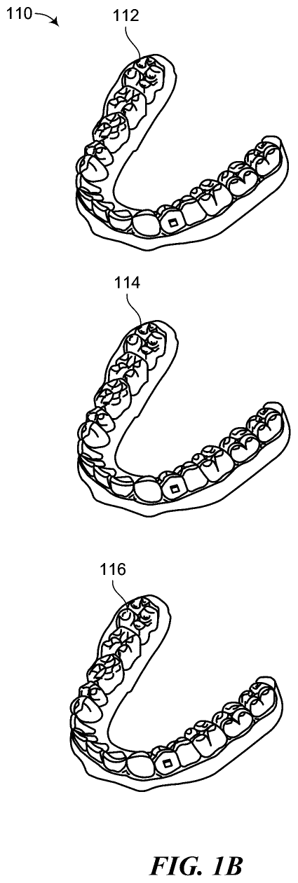

FIG. 2 illustrates a method 200 for designing an orthodontic appliance to be produced by direct fabrication, in accordance with embodiments. The method 200 can be applied to any embodiment of the orthodontic appliances described herein. Some or all of the steps of the method 200 can be performed by any suitable data processing system or device, e.g., one or more processors configured with suitable instructions.

In step 210, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In step 220, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as Xray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In step 230, an arch or palate expander design for an orthodontic appliance configured to produce the force system is determined. Determination of the arch or palate expander design, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA(Abaqus) software products from Dassault Systemes of Waltham, MA.

Optionally, one or more arch or palate expander designs can be selected for testing or force modeling. As noted above, a desired tooth movement, as well as a force system required or desired for eliciting the desired tooth movement, can be identified. Using the simulation environment, a candidate arch or palate expander design can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In step 240, instructions for fabrication of the orthodontic appliance incorporating the arch or palate expander design are generated. The instructions can be configured to control a fabrication system or device in order to produce the orthodontic appliance with the specified arch or palate expander design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Method 200 may comprise additional steps: 1) The upper arch and palate of the patient is scanned intraorally to generate three-dimensional data of the palate and upper arch; 2) The three-dimensional shape profile of the appliance is determined to provide a gap and teeth engagement structures as described herein.

Although the above steps show a method 200 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 200 may be performed with any suitable fabrication system or device, such as the embodiments described herein. Some of the steps may be optional, and the order of the steps can be varied as desired.

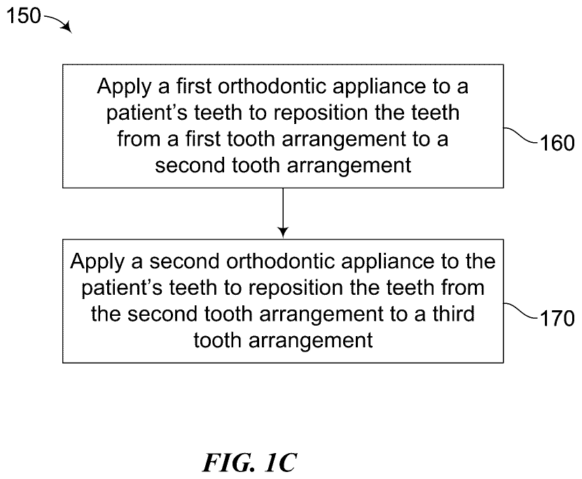

FIG. 3 illustrates a method 300 for digitally planning an orthodontic treatment and/or design or fabrication of an appliance, in accordance with embodiments. The method 300 can be applied to any of the treatment procedures described herein and can be performed by any suitable data processing system.

In step 310, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In step 320, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 330, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 3, design and/or fabrication of an orthodontic appliance, and perhaps a particular orthodontic treatment, may include use of a representation of the patient's teeth (e.g., receive a digital representation of the patient's teeth 310), followed by design and/or fabrication of an orthodontic appliance based on a representation of the patient's teeth in the arrangement represented by the received representation.

### On-Track Treatment

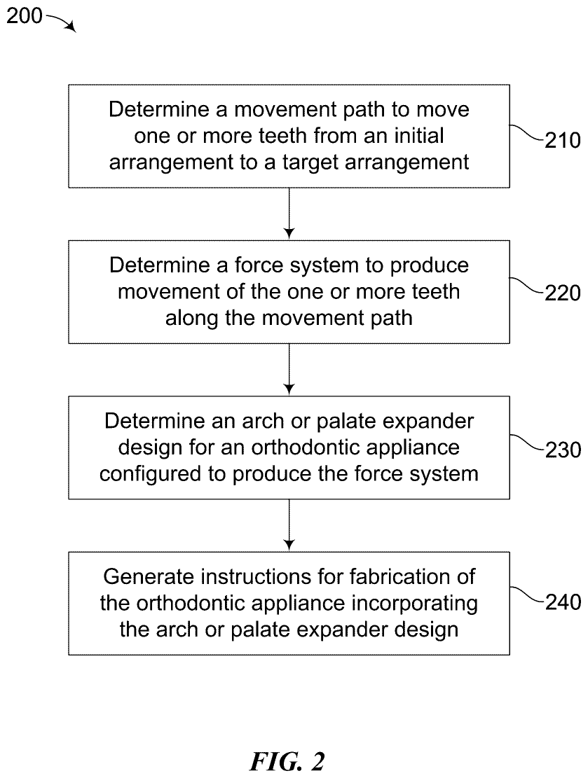

Referring to FIG. 4, a process 400 according to the present disclosure is illustrated. Individual aspects of the process are discussed in further detail below. The process includes receiving information regarding the orthodontic condition of the patient and/or treatment information (402), generating an assessment of the case (404), and generating a treatment plan for repositioning a patient's teeth (406). Briefly, a patient/treatment information includes data comprising an initial arrangement of the patient's teeth, which includes obtaining an impression or scan of the patient's teeth prior to the onset of treatment and can further include identification of one or more treatment goals selected by the practitioner and/or patient. A case assessment can be generated (404) so as to assess the complexity or difficulty of moving the particular patient's teeth in general or specifically corresponding to identified treatment goals, and may further include practitioner experience and/or comfort level in administering the desired orthodontic treatment. In some cases, however, the assessment can include simply identifying particular treatment options (e.g., appointment planning, progress tracking, etc.) that are of interest to the patient and/or practitioner. The information and/or corresponding treatment plan includes identifying a final or target arrangement of the patient's teeth that is desired, as well as a plurality of planned successive or intermediary tooth arrangements for moving the teeth along a treatment path from the initial arrangement toward the selected final or target arrangement.

The process further includes generating customized treatment guidelines (408). The treatment plan may include multiple phases of treatment, with a customized set of treatment guidelines generated that correspond to a phase of the treatment plan. The guidelines can include detailed information on timing and/or content (e.g., specific tasks) to be completed during a given phase of treatment, and can be of sufficient detail to guide a practitioner, including a less experienced practitioner or practitioner relatively new to the particular orthodontic treatment process, through the phase of treatment. Since the guidelines are designed to specifically correspond to the treatment plan and provide guidelines on activities specifically identified in the treatment information and/or generated treatment plan, the guidelines can be customized. The customized treatment guidelines are then provided to the practitioner so as to help instruct the practitioner as how to deliver a given phase of treatment. As set forth above, appliances can be generated based on the planned arrangements and can be provided to the practitioner and ultimately administered to the patient (410). The appliances can be provided and/or administered in sets or batches of appliances, such as 2, 3, 4, 5, 6, 7, 8, 9, or more appliances, but are not limited to any particular administrative scheme. Appliances can be provided to the practitioner concurrently with a given set of guidelines, or appliances and guidelines can be provided separately.

After the treatment according to the plan begins and following administration of appliances to the patient, treatment progress tracking, e.g., by teeth matching, is done to assess a current and actual arrangement of the patient's teeth compared to a planned arrangement (412). If the patient's teeth are determined to be “on-track” and progressing according to the treatment plan, then treatment progresses as planned and treatment progresses to the next stage of treatment (414). If the patient's teeth have substantially reached the initially planned final arrangement, then treatment progresses to the final stages of treatment (414). Where the patient's teeth are determined to be tracking according to the treatment plan, but have not yet reached the final arrangement, the next set of appliances can be administered to the patient.

The threshold difference values of a planned position of teeth to actual positions selected as indicating that a patient's teeth have progressed on-track are provided below in Table 3. If a patient's teeth have progressed at or within the threshold values, the progress is considered to be on-track. If a patient's teeth have progressed beyond the threshold values, the progress is considered to be off-track.

TABLE 3

| Type Movement | Difference Actual/Planned | |
|---|---|---|
| Rotations | | |
| Upper Central Incisors | 9 | degrees |
| Upper Lateral Incisors | 11 | degrees |
| Lower Incisors | 11 | degrees |
| Upper Cuspids | 11 | degrees |
| Lower Cuspids | 9.25 | degrees |
| Upper Bicuspids | 7.25 | degrees |
| Lower First Bicuspid | 7.25 | degrees |
| Lower Second Bicuspid | 7.25 | degrees |
| Molars | 6 | degrees |
| Extrusion | | |
| Anterior | 0.75 | mm |
| Posterior | 0.75 | mm |
| Intrusion | | |
| Anterior | 0.75 | mm |
| Posterior | 0.75 | mm |
| Angulation | | |
| Anterior | 5.5 | degrees |
| Posterior | 3.7 | degrees |
| Inclination | | |
| Anterior | 5.5 | degrees |
| Posterior | 3.7 | degrees |
| Translation | | |
| BL Anterior | 0.7 | mm |
| BL Posterior Cuspids | 0.9 | mm |
| MD Anterior | 0.45 | mm |
| MD Cuspids | 0.45 | mm |
| MD Posterior | 0.5 | mm |

The patient's teeth are determined to be on track by comparison of the teeth in their current positions with teeth in their expected or planned positions, and by confirming the teeth are within the parameter variance disclosed in Table 3. If the patient's teeth are determined to be on track, then treatment can progress according to the existing or original treatment plan. For example, a patient determined to be progressing on track can be administered one or more subsequent appliances according to the treatment plan, such as the next set of appliances. Treatment can progress to the final stages and/or can reach a point in the treatment plan where bite matching is repeated for a determination of whether a patient's teeth are progressing as planned or if the teeth are off track.

In some embodiments, as further disclosed herein, this disclosure provides methods of treating a patient using a 3D) printed orthodontic appliance. As a non-limiting example, orthodontic appliances comprising crystalline domains, polymer crystals, and/or materials that can form crystalline domains or polymer crystals can be 3D printed and used to reposition a patient's teeth. In certain embodiments, the method of repositioning a patient's teeth (or, in some embodiments, a singular tooth) comprises: generating a treatment plan for the patient, the plan comprising a plurality of intermediate tooth arrangements for moving teeth along a treatment path from an initial arrangement toward a final arrangement; producing a 3D printed orthodontic appliance; and moving on-track, with the orthodontic appliance, at least one of the patient's teeth toward an intermediate arrangement or a final tooth arrangement. In some embodiments, producing the 3D printed orthodontic appliance uses the crystallizable resins disclosed further herein. On-track performance can be determined, e.g., from TABLE 3, above.

In some embodiments, the method further comprises tracking the progression of the patient's teeth along the treatment path after administration of the orthodontic appliance. In certain embodiments, the tracking comprises comparing a current arrangement of the patient's teeth to a planned arrangement of the teeth. As a non-limiting example, following the initial administration of the orthodontic appliance, a period of time passes (e.g., two weeks), a comparison of the now-current arrangement of the patient's teeth (i.e., at two weeks of treatment) can be compared with the teeth arrangement of the treatment plan. In some embodiments, the progression can also be tracked by comparing the current arrangement of the patient's teeth with the initial configuration of the patient's teeth. The period of time can be, for example, greater than 3 days, greater than 4 days, greater than 5 days, greater than 6 days, greater than 7 days, greater than 8 days, greater than 9 days, greater than 10 days, greater than 11 days, greater than 12 days, greater than 13 days, greater than 2 weeks, greater than 3 weeks, greater than 4 weeks, or greater than 2 months. In some embodiments, the period of time can be from at least 3 days to at most 4 weeks, from at least 3 days to at most 3 weeks, from at least 3 days to at most 2 weeks, from at least 4 days to at most 4 weeks, from at least 4 days to at most 3 weeks, or from at least 4 days to at most 2 weeks. In certain embodiments, the period of time can restart following the administration of a new orthodontic appliance.

In some embodiments, greater than 50%, greater than 55%, greater than 60%, greater than 65%, greater than 70%, greater than 75%, greater than 80%, greater than 85%, greater than 90%, greater than 91%, greater than 92%, greater than 93%, greater than 94%, greater than 95%, greater than 96%, greater than 97%, greater than 98%, or greater than 99% of the patient's teeth are on track with the treatment plan after a period of time of using an orthodontic appliance as disclosed further herein. In some embodiments, the period of time is 3 days, 4 days, 5 days, 6 days, 7 days, 8 days, 9 days, 10 days, 11 days, 12 days, 13 days, 2 weeks, 3 weeks, 4 weeks, or greater than 4 weeks.

As disclosed further herein, orthodontic appliances disclosed herein have advantageous properties, such as increased durability, and an ability to retain resilient forces to a patient's teeth for a prolonged period of time. In some embodiments of the method disclosed above, the 3D printed orthodontic appliance has a retained repositioning force (i.e., the repositioning force after the orthodontic appliance has been applied to or worn by the patient over a period of time), and the retained repositioning force to at least one of the patient's teeth after the period of time is at least 10%, at least 20%, at least 30%, at least 40%, at least 50%, at least 55%, at least 60%, at least 65%, at least 70%, at least 75%, at least 80%, at least 85%, at least 90%, at least 95%, at least 96%, at least 97%, at least 98%, or at least 99% of the repositioning force initially provided to the at least one of the patient's teeth (i.e., with initial application of the orthodontic appliance). In some embodiments, the period of time is 1 day, 2 days, 3 days, 4 days, 5 days, 6 days, 7 days, 8 days, 9 days, 10 days, 11 days, 12 days, 13 days, 2 weeks, 3 weeks, 4 weeks, or greater than 4 weeks. In some embodiments, the repositioning force applied to at least one of the patient's teeth is present for a time period of less than 24 hours, from about 24 hours to about 2 months, from about 24 hours to about 1 month, from about 24 hours to about 3 weeks, from about 24 hours to about 14 days, from about 24 hours to about 7 days, from about 24 hours to about 3 days, from about 3 days to about 2 months, from about 3 days to about 1 month, from about 3 days to about 3 weeks, from about 3 days to about 14 days, from about 3 days to about 7 days, from about 7 days to about 2 months, from about 7 days to about 1 month, from about 7 days to about 3 weeks, from about 7 days to about 2 weeks, or greater than 2 months. In some embodiments, the repositioning force applied to at least one of the patient's teeth is present for about 24 hours, for about 3 days, for about 7 days, for about 14 days, for about 2 months, or for more than 2 months.

In some embodiments, the orthodontic appliances disclosed herein can provide on-track movement of at least one of the patient's teeth. On-track movement has been described further herein, e.g., at Table 3. In some embodiments, the orthodontic appliances disclosed herein can be used to achieve on-track movement of at least one of the patient's teeth to an intermediate tooth arrangement. In some embodiments, the orthodontic appliances disclosed herein can be used to achieve on-track movement of at least one of the patient's teeth to a final tooth arrangement.

In some embodiments, prior to moving, with the orthodontic appliance, at least one of the patient's teeth toward an intermediate arrangement or a final tooth arrangement, the orthodontic appliance has characteristics which are retained following the use of the orthodontic appliance. In some embodiments, prior to the moving step, the orthodontic appliance comprises a first flexural modulus. In certain embodiments, after the moving step, the orthodontic appliance comprises a second flexural modulus. In some embodiments, the second flexural modulus is at least 99%, at least 98%, at least 97%, at least 96%, at least 95%, at least 94%, at least 93%, at least 92%, at least 91%, at least 90%, at least 85%, at least 80%, at least 75%, at least 70%, at least 65%, at least 60%, at least 50%, or at least 40% of the first flexural modulus. In some embodiments, the second flexural modulus is greater than 50% of the first flexural modulus. In some embodiments, this comparison is performed following a period of time in which the appliance is applied. In some embodiments, the period of time is 3 days, 4 days, 5 days, 6 days, 7 days, 8 days, 9 days, 10 days, 11 days, 12 days, 13 days, 2 weeks, 3 weeks, 4 weeks, or greater than 4 weeks.

In some embodiments, prior to the moving step, the orthodontic appliance comprises a first elongation at break. In certain embodiments, after the moving step, the orthodontic appliance comprises a second elongation at break. In some embodiments, the second elongation at break is at least 99%, at least 98%, at least 97%, at least 96%, at least 95%, at least 94%, at least 93%, at least 92%, at least 91%, at least 90%, at least 85%, at least 80%, at least 75%, at least 70%, at least 65%, at least 60%, at least 50%, or at least 40% of the first elongation at break. In some embodiments, the second elongation at break is greater than 50% of the first elongation at break. In some embodiments, this comparison is performed following a period of time in which the appliance is applied. In some embodiments, the period of time is 3 days, 4 days, 5 days, 6 days, 7 days, 8 days, 9 days, 10 days, 11 days, 12 days, 13 days, 2 weeks, 3 weeks, 4 weeks, or greater than 4 weeks.

As provided herein, the methods disclosed can use the orthodontic appliances further disclosed herein. The orthodontic appliances can be directly fabricated using, e.g., the crystallizable resins disclosed herein. In certain embodiments, the direct fabrication comprises cross-linking the crystallizable resin.

The appliances formed from the crystallizable resins disclosed herein provide improved durability, strength, and flexibility, which in turn improve the rate of on-track progression in treatment plans. In some embodiments, greater than 60%, greater than 70%, greater than 80%, greater than 90%, or greater than 95% of patients treated with the orthodontic appliances disclosed herein (e.g., an aligner) are classified as on-track in a given treatment stage. In certain embodiments, greater than 60%, greater than 70%, greater than 80%, greater than 90%, or greater than 95% of patients treated with the orthodontic appliances disclosed herein (e.g., an aligner) have greater than 50%, greater than 55%, greater than 60%, greater than 65%, greater than 70%, greater than 75%, greater than 80%, greater than 85%, greater than 90%, or greater than 95% of their tooth movements classified as on-track.

As disclosed further herein, the cured polymeric material contains favorable characteristics that, at least in part, stem from the presence of polymeric crystals. These cured polymeric materials can have increased resilience to damage, can be tough, and can have decreased water uptake when compared to similar polymeric materials. The cured polymeric materials can be used for devices within the field of orthodontics, as well as outside the field of orthodontics. For example, the cured polymeric materials disclosed herein can be used to make devices for use in aerospace applications, automobile manufacturing, the manufacture of prototypes, and/or devices for use in durable parts production.

### EXPERIMENTAL METHODS

All chemicals were purchased from commercial sources and were used without further purification, unless otherwise stated.

¹H NMR and ¹³C NMR spectra were recorded on a BRUKER AC-E-200 FT-NMR spectrometer or a BRUKER Advance DRX-400 FT-NMR spectrometer. The chemical shifts are reported in ppm (s: singlet, d: doublet, t: triplet, q: quartet, m: multiplet). The solvents used were deuterated chloroform (CDCl₃, 99.5% deuteration) and deuterated DMSO (d₆-DMSO, 99.8% deuteration).

In some embodiments, the stress relaxation of a material or device can be measured by monitoring the time-dependent stress resulting from a steady strain. The extent of stress relaxation can also depend on the temperature, relative humidity and other applicable conditions (e.g., presence of water). In embodiments, the test conditions for stress relaxation are a temperature of 37±2° C. at 100% relative humidity or a temperature of 37±2° C. in water.

The dynamic viscosity of a fluid indicates its resistance to shearing flows. The SI unit for dynamic viscosity is the Poiseuille (Pa·s). Dynamic viscosity is commonly given in units of centipoise, where 1 centipoise (cP) is equivalent to 1 mPa·s. Kinematic viscosity is the ratio of the dynamic viscosity to the density of the fluid; the SI unit is m2/s. Devices for measuring viscosity include viscometers and rheometers. For example, an MCR 301 rheometer from Anton Paar may be used for rheological measurement in rotation mode (PP-25, 50 s-1, 50-115° C., 3° C./min).

Determining the water content when fully saturated fat use temperature can comprise exposing the polymeric material to 100% humidity at the use temperature (e.g., 40° C.) for a period of 24 hours, then determining water content by methods known in the art, such as by weight.

In some embodiments, the presence of a crystalline phase and an amorphous phase provide favorable material properties to the polymeric materials. Property values of the cured polymeric materials can be determined, for example, by using the following methods:
flexural modulus, remaining flexural stress, and stress relaxation properties can be assessed using an RSA-G2 instrument from TA Instruments, with a 3-point bending, according to ASTM D790; for example, stress relaxation can be measured under static conditions at a loading rate of 32 mm/min and a strain of 5% at 30° C. and submerged in water, and reported as the remaining load after 24 hours, as either the percent (%) of initial load, and/or in MPa;
storage modulus can be measured at 37° C. and is reported in MPa;
Tg of the cured polymeric material can be assessed using dynamic mechanical analysis (DMA) and is provided herein as the tan 6 peak;
tensile modulus, tensile strength, elongation at yield and elongation at break can be assessed according to ISO 527-2 5B; and tensile strength at yield, elongation at break, tensile strength, and Young's modulus can be assessed according to ASTM D1708;
molecular weight can be measured by size exclusion chromatography or gel permeation chromatography.

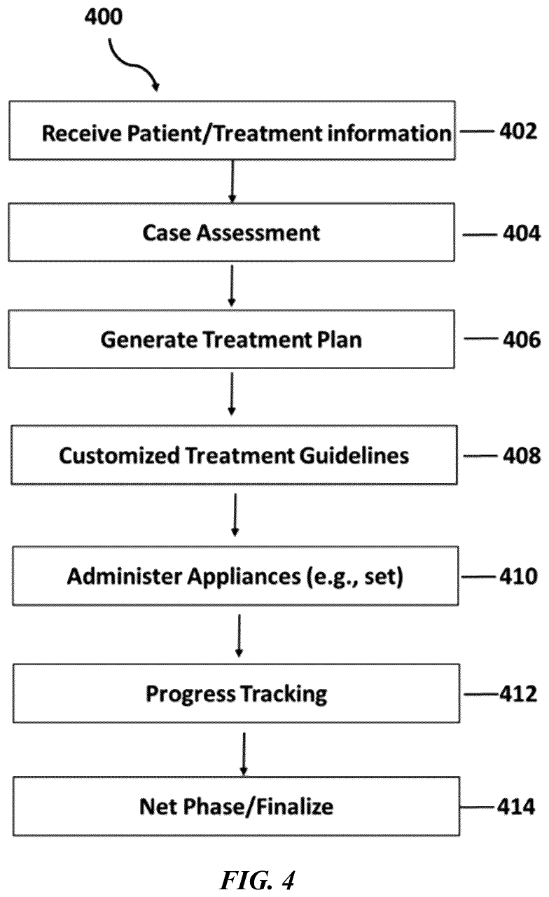

Additive manufacturing or 3D printing processes for generating a device herein (e.g., an orthodontic appliance) can be conducted using a Hot Lithography apparatus prototype from Cubicure (Vienna, Austria), which can substantially be configured as schematically shown in FIG. 6. In such cases, a photo-curable composition (e.g., resin) according to the present disclosure can be filled into the transparent material vat of the apparatus shown in FIG. 6, which vat can be heated to 90-110° C. The building platform can be heated to 90-110° C., too, and lowered to establish holohedral contact with the upper surface of the curable composition. By irradiating the composition with 375 nm UV radiation using a diode laser from Soliton, which can have an output power of 70 mW, which can be controlled to trace a predefined prototype design, and alternately raising the building platform, the composition can be cured layer by layer by a photopolymerization process according to the disclosure, resulting in a polymeric material according to present disclosure.

### EXAMPLES

The following examples are given for the purpose of illustrating various embodiments of the invention and are not meant to limit the present disclosure in any fashion. The present examples, along with the methods described herein are presently representative of some embodiments, are exemplary, and are not intended as limitations on the scope of the invention. Changes therein and other uses which are encompassed within the spirit of the invention as defined by the scope of the claims will occur to those skilled in the art.

### Example 1

### Synthesis of Mono-Methacrylate Liquid Crystalline Compound I-8a

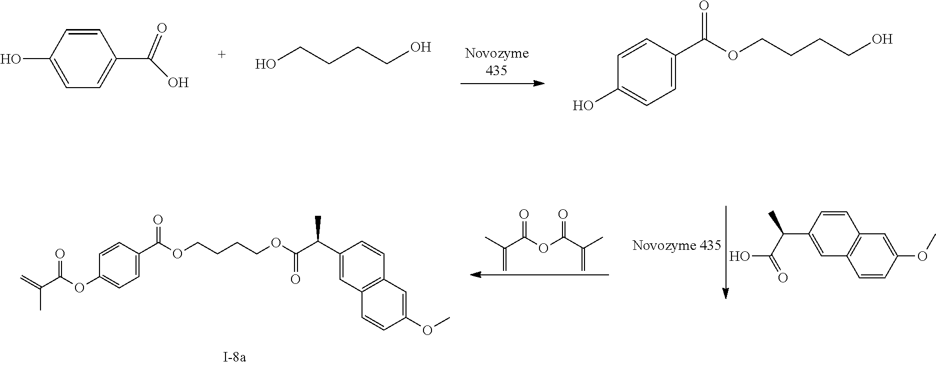 [SMILES: O=C(O)c1ccc(O)cc1 ; OCCCCO ; O=C(OCCCCO)c1ccc(O)cc1 ; C=C(C)C(=O)Oc1ccc(C(=O)OCCCCOC(=O)[C@@H](C)c2ccc3cc(OC)ccc3c2)cc1 ; C=C(C)C(=O)OC(=O)C(=C)C ; COc1ccc2cc([C@H](C)C(=O)O)ccc2c1]

Liquid crystalline methacrylate I-8a can be synthesized using a general procedure described herein. For example, a large excess of butane diol is reacted with aromatic carboxylic acid in solvent such as dichloromethane at room temperature for 24 hours using up to 5 wt % of catalyst. The diol product, 4-hydroxybutyl 4-hydroxybenzoate, is isolated via chromatography, and then reacted with a large excess of a second carboxylic acid under the same conditions as above. After isolation using column chromatography, the product phenol is reacted with a slight excess of methacrylic anhydride in pyridine at room temperature initially by slow addition, and then heated to 60° C. for 2 hours. Acidic water and dichloromethane mixtures can be used to remove the pyridine, and potassium carbonate in water and dichloromethane can remove methacrylic acids. The final liquid crystalline monomer I-8a can be columned to obtain a white solid material.

### Example 2

### Synthesis of Side-Chain Liquid Crystalline Polymer II-1

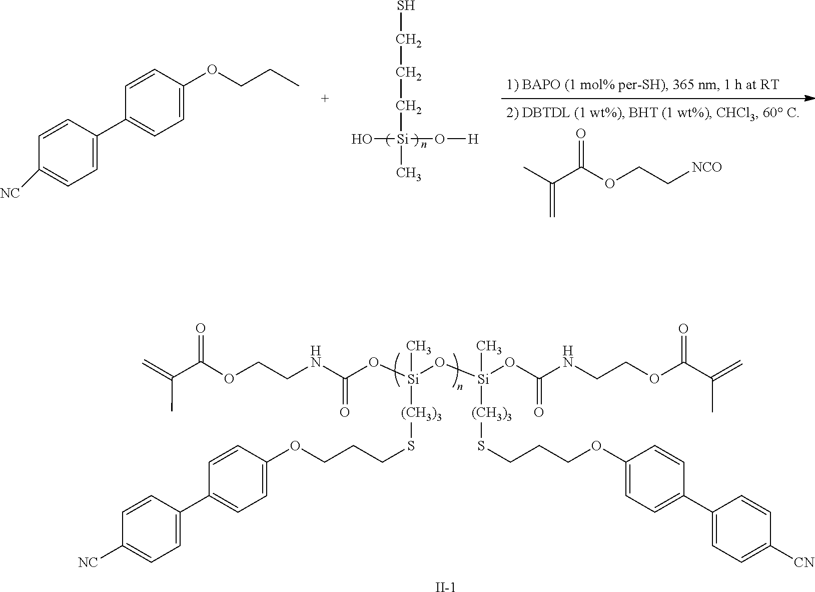

The starting siloxane diol is reacted to the allyl ether using a free radical initiator (photo or thermal). After the free radical reaction, the liquid crystal side chains are formed and methacrylic acid or methacryloyl chloride can be used to functionalize the silanols.

### Example 3

### Synthesis of Side-Chain Liquid Crystalline Polymer II-2

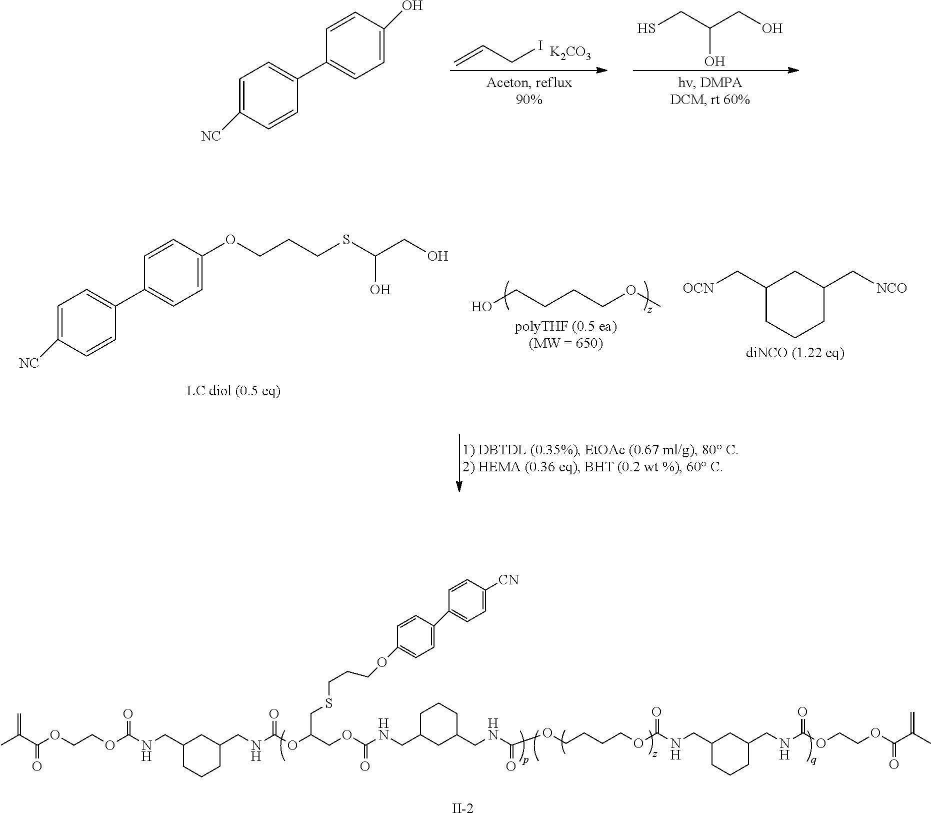

### Example 4

### Synthesis of Compound III-1

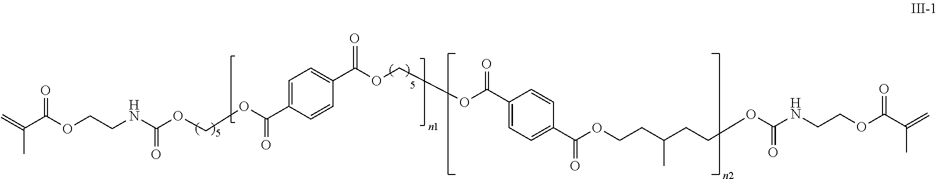

In a round-bottom flask equipped with a magnetic stir bar was dissolved poly(methylpentane-co-pentane terephthalate) (1:16 ratio, 4000 g/L) (5.069 g) and dichloromethane (35 g). Then, 2-isocyanatoethyl methacrylate (0.506 g) was added to the flask, followed by adding dibutyltin dilaurate (DBTDL) catalyst (1 wt %). The reaction mixture was reacted for 4 days.

### Example 5

### Formulation of Compound III-1

In 2 grams of Compound III-1 (pentane:methylpentane; 16:1; Mn: 4000 g/ml), was added 1 wt % TPO photoinitiator. The mixture was heated, casted and cured in Dymax chamber 4× (90 sec) flipping each time and on a hot plate at 140° C.

Upon cooling after curing, the sample was removed from between the glass slides and annealed to 90° C. for 2 or 3 minutes. Then sample went from rubbery and slightly hazy to cloudy and stiff. When cooled back to room temperature, the sample had a stiff feel with good spring back. DMA characterization and DMA stress relaxation were measured and results.

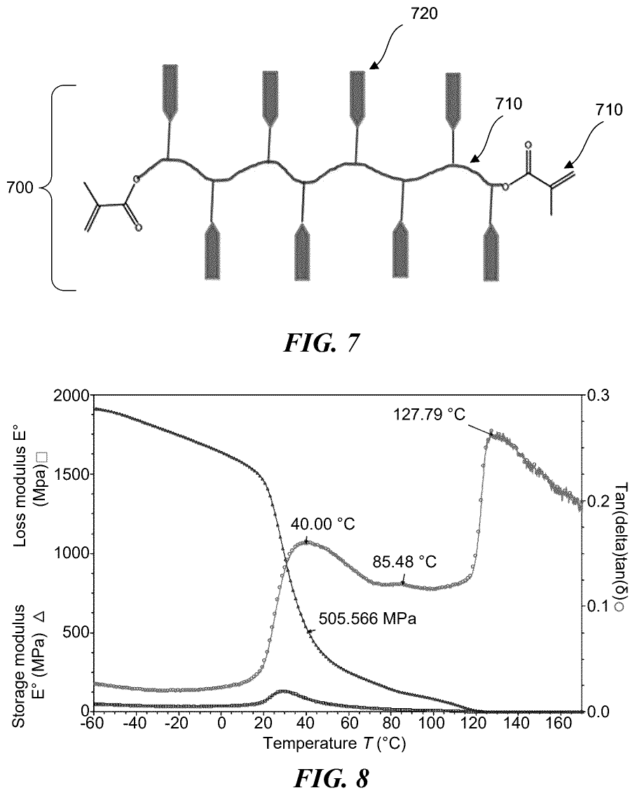

As shown in FIG. 9, DMA characterization showed that Tg from methylpentane segment is around 40° C., and Tm of pentaneterephthalate segment is around 127° C.

Storage Modulus is 505 MPa at 40° C.

Flexural modulus remaining after 24 hours is 90 MPa (3 point bend with 5% strain in water and run for 24 hours).

### Example 6

TABLE 4
FORMULATION OF COMPOUND III-1

| | | | Weight | Molecular |
|---|---|---|---|---|
| | Amount | | percent | weight |
| Component | (g) | Functionality | (wt %) | (g/mole) |
| Compound III-1 | 1.75 | 2 | 89 | 4000 |
| (poly(methylpentane-co-pentane terephthalate) | | | | |
| 1:16 ratio bis(urethane methacrylate) | | | | |
| SMA | 0.175 | 1 | 10 | |
| TPO | 0.014 | | 1 | |

The mixture was heated to 140° C. so that all components were melted. A white solid was formed as the mixture was cooled down. The solid was placed on a glass slide at 140° C. The solid was then melted and became clear. The spacers (800 micron) and a preheated glass slide were placed over the glass slide with the melted resin to form a glass sandwich with the clear resin in between. The glass sandwich was placed onto a hot metal plate and then transferred into Dymax chamber and cured 4× (90 sec) flipping each time. The glass sandwich was removed from the chamber and cooled to room temperature, and then the sample was removed from the glass sandwich. The sample was clear and rubbery. After letting it sit for 3 days, the sample hardened with good modulus and transparency (slight haze).

### Example 7

### Formulation of Compound III-1

In 2 g of compound III-1 (pentane:methylpentane; 98:2; Mn of 7.5 k g/ml) with dimethacrylate functionality (between 80% to 90% end group functionalization) was added 1 wt % TPO photoinitiator. The mixture was heated, casted and cured in Dymax chamber 4× (90 sec) flipping each time and then placed on a hot plate at 120° C. The sample was removed from Dymax and let cool to room temperature. A clear tough slow to spring back rubbery material was obtained. Annealing at 80° C. (below the Tm of the polymer's crystalline phase) for 30 min resulted in a cloudy material from the induced crystallization.

### Example 8

TABLE 5
FORMULATION OF COMPOUND III-1

| | | | Weight | |
|---|---|---|---|---|
| | | | percent | Molecular weight |
| Component | Amount (g) | Functionality | (wt %) | (g/mole) |
| Compound III-1 | 1 | 2 | 49.5 | 25,000 |
| (poly(pentane-co-methylpentane terephthalate) 53:1 molar ratio diacrylate | | | | |
| cyanobiphenyl methacrylate | 1 | 1 | 49.5 | 232.28 |
| 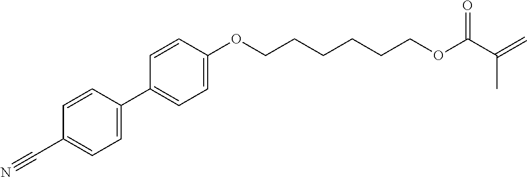 [SMILES: C=C(C)C(=O)OCCCCCCOc1ccc(-c2ccc(C#N)cc2)cc1] | | | | |
| TPO | 0.020 | | 1 | |

Compound III-1, cyanobiphenyl methacrylate and TPO were manually mixed to provide a powered mixture. The powdered mixture was heated to 150° C. till all components were melted. A clear yellowish viscous liquid formed and slowly crystallized as it cooled. The resulting solid was placed on a slide glass at 140° C. (on a hot metal plate), and the solid was melted by 110° C. After placing spacers (0.8 mm) and a top preheated glass slide over the glass slide with the liquid resin to form a sample sandwiched between two glass slides, the sample was placed on a hot metal plate (120° C.) in a Dymax chamber and cured 4× (90 sec) flipping each time (temperature of sample was 100° C. during curing). Sample turned white during curing in the Dymax chamber. The sample was taken out and reheated to melt it. With heating, the sample material transitioned from cloudy to clear at around 95° C.; while at this temperature, the sample material slowly turned cloudy in a different way. This demonstrated the Tm of the liquid crystalline cyanobiphenyl polymer around 95° C., and while in the melted state, the terephthalate chains were able to crystallize (Tm around 130° C.). Accordingly, two different crystalline domains may be obtained in a single sample with proper annealing. After annealing further at 100° C., the sample material became brittle as more of the terephthalate crystallized.

### Example 9

TABLE 6
FORMULATION OF COMPOUND III-2

| | | | Weight | Molecular |
|---|---|---|---|---|
| | Amount | Func- | percent | weight |
| Component | (g) | tionality | (wt %) | (g/mole) |
| Compound III-2 | 2 | 2 | 98.5 | 7532 |
| (poly(decane-co- | | | | |
| methylpentane | | | | |
| terephthalate) | | | | |
| 23:1 molar | | | | |
| ratio diacrylate) | | | | |
| TPO | 0.03 | | 1.5 | |

A mixture of compound III-1 and TPO was heated to 140° C. to melt the mixture, affording a clear slightly yellow liquid with lowish viscosity. The liquid was then speed mixed (FlackTek Speed Mixer) and placed in glass sandwich with spacers (800 microns) and cured in Dymax for 3×90 sec on each side. Crystallization occurred around 60° C. Upon reheating and annealing, a crystalline domain with a higher melting temperature greater than 110° C. was obtained. The stress relaxation test results are shown in FIG. 9.

Tensile 1 mm/min=Stress @yield=16 MPa, Stress @break=19 MPa, Elongation to Yield=25%, Elongation to Break=172%, Young Mod=100 MPa

A remaining flexural Modulus (after 24 hrs in 40 C water at 5% strain on 3 point bend)=67 MPa (Start 260 MPa, 25% stress retention).

### Example 10

TABLE 7
FORMULATION OF COMPOUND III-6

| | | | Weight | Molecular |
|---|---|---|---|---|
| | Amount | Func- | percent | weight |
| Component | (g) | tionality | (wt %) | (g/mole) |
| Compound III-6 | 3.0359 | 2 | 89.6 | 5264 |
| (poly(pentane terephthalate- | | | | |
| co-PTHF(1000) 15:1 molar | | | | |
| ratio diacrylate - random | | | | |
| copolymer) | | | | |
| SMA | 0.3204 | 1 | 9.4 | |
| TPO | 0.0307 | | 0.9 | |

A powdered mixture was heated to 150° C. (in the oven) for 10 min, then speed mixed at 3500 rpm for 1 min. After leaving in oven for 10 min, the liquid was placed onto a hot glass plate, and with 800 micron spacer, the liquid was sandwiched between two glass plates (both glass plates were heated to 120 C on hot metal plate), then transferred to Dymax chamber and cured in the chamber, 1×90 sec, flip sandwich, 2×90 sec cures, flip, 1×90 sec cure. The uncured resin spread easily while hot, but was thixotropic in that it would hold its form while melted. This was a clear resin when uncured and hot, and cured clear. The sample was placed in freezer for 30 min to induce crystallization, and let sit for 3 days at room temperature. Material became slightly hazy and stiffer, indicating that crystallization had occurred.

Final Flex Modulus=24 hr, 37 C in water (3 point bend at 5% strain)=23 MPa.

### Example 11

TABLE 8
FORMULATION OF COMPOUND III-6

| | | | Weight | Molecular |
|---|---|---|---|---|
| | Amount | | percent | weight |
| Component | (g) | Functionality | (%) | (g/mole) |
| Compound III-6 | 3.5508 | 2 | 90.3 | 11,521 |
| (poly(pentane terephthalate-co-PTHF(2800) | | | | |
| 38:1 molar ratio diacrylate - random | | | | |
| copolymer) | | | | |
| SMA | 0.3407 | 1 | 8.7 | |
| TPO | 0.0386 | | 1 | |

A powdered mixture was heated to 150° C. (in the oven) for 10 min, then speed mixed (FlackTek Speed Mixer) at 3500 rpm for 1 min. After mixing, the mixture was placed back into an oven to remelt it (10 min), then poured onto a hot glass plate, and with 800 micron spacer, the liquid was sandwiched between two glass plates (both glass plates were heated to 120° C.), and then transferred to Dymax chamber and cured in the chamber (on a hot metal plate that had been heated to 120° C.), 1×90 sec, flip sandwich, 2×90 sec cures, flip, 1×90 sec cure. Uncured resin was a clear very viscous fluid while hot. Upon curing, a very pliable rubbery material was formed. The material started to turn hazy after being cooled to 40° C. The material was very rubbery with an elongation to break greater than 300%. The material stiffens up slightly overnight.

Final flexural modulus (Stress Relaxation)=24 hr, 37 C in water (3 point bend at 5% strain)=28 MPa.

### Example 12

TABLE 9
FORMULATION OF COMPOUND III-6

| | | | Weight | Molecular |
|---|---|---|---|---|
| | Amount | Func- | percent | weight |
| Component | (g) | tionality | (wt %) | (g/mole) |
| Compound III-6 | 2.0541 | 2 | 47.8 | 5,264 |
| (poly(pentane terephthalate-co-PTHF(1000) | | | | |
| 15:1 molar ratio diacrylate - random | | | | |
| copolymer) | | | | |
| Compound III-6 | 2.1131 | 2 | 49.1 | 4,534 |
| (poly(pentane terephthalate-co-PTHF(1000) | | | | |
| 15:1 molar ratio diallyl ether - random | | | | |
| copolymer) | | | | |
| 1,10-decane dithiol (98% pure) | 0.1126 | 2 | 2.6 | 206.41 |
| TPO | 0.0205 | | 0.5 | |

A powdered mixture was heated to 150° C. (in the oven) for 10 min, then speed mixed at 3500 rpm for 1 min (FlackTek Speed Mixer). After mixing, the mixture was placed back into an oven to remelt it (10 min), then the liquid was placed onto a hot glass plate, and with 800 micron spacer, the liquid was sandwiched between two glass plates (both glass plates were heated to 120° C. on a hot metal plate), and then transferred to Dymax chamber and cured in the chamber while still on the hot metal plate, 1×90 sec, flip sandwich, 2×90 sec cures, flip, 1×90 sec cure. The uncured resin was a clear and very viscous fluid while hot. Upon curing, a very pliable rubbery material was formed. The material started to turn hazy after being cooled to 40° C. The material was very rubbery with a elongation to break greater than 300%.

Final flexural modulus (Stress Relaxation) 24 hr, 37 C in water (3 point bend at 5% strain) 61 MPa.

### Example 13

TABLE 10
EFFECT OF DIOLS ON PROPERTIES OF COMPOUNDS OF STRUCTURE (III)

| | | Mn | Mw | | |
|---|---|---|---|---|---|
| # | Diester-Diols | (g/mol) | (g/mol) | PDI | Tm (° C.) |
| 1 | Diethyl terephthalate (1.0 mol) | 11 k | 22 k | 1.9 | 127 |
| | 1,4-butanediol (0.2 mol) | | | | |
| | 1,6-hexanediol (0.8 mol) | | | | |
| 2 | Diethyl terephthalate (1.0 mol) | 7 k | 21 k | 2.9 | 123, 166 |
| | 1,4-butanediol (0.3 mol) | | | | |
| | 1,6-hexanediol (0.7 mol) | | | | |
| 3 | Diethyl terephthalate (1.0 mol) | 6 k | 18 k | 2.8 | 130 |
| | 1,4-butanediol (0.4 mol) | | | | |
| | 1,6-hexanediol (0.6 mol) | | | | |
| 4 | Diethyl terephthalate (1.0 mol) | 5.8 k | 30 k | 5.0 | 141 |
| | 1,4-butanediol (0.5 mol) | | | | |
| | 1,6-hexanediol (0.5 mol) | | | | |
| 5 | Diethyl terephthalate (1.0 mol) | 10 k | 20 k | 2.0 | 169 |
| | 1,4-butanediol (0.6 mol) | | | | |
| | 1,6-hexanediol (0.4 mol) | | | | |
| 6 | Diethyl terephthalate (1.0 mol) | 8.7 k | 16 k | 1.9 | 183 |
| | 1,4-butanediol (0.7 mol) | | | | |
| | 1,6-hexanediol (0.3 mol) | | | | |

The effect of different diols on the crystallinity of compounds of structure (III) was studied by polymerizing diethyl terepthalate with 1,4-butanediol and 1,6-hexanediol and the results are summarized in Table 10. As shown in Table 10, increasing the amount of 1,4-butanediol raises the melting temperature (Tm) of the resulting polymer, indicating that a shorter aliphatic diol enhances crystallinity.

### Example 14

TABLE 11
EFFECT OF DIESTERS ON PROPERTIES
OF COMPOUNDS OF STRUCTURE (III)

| | | Mn | Mw | | Tm | Tg | Solubility in |
|---|---|---|---|---|---|---|---|
| # | Diester-Diols | (g/mol) | (g/mol) | PDI | (° C.) | (° C.) | chloroform |
| 1 | Di-tert-butyl terephthalate | 10k | 22k | 2.1 | 135 | 16 | Partially soluble |
| | (1.0 mol) | | | | | | |
| | 1,5-pentanediol (0.5 mol) | | | | | | |
| | 1,4-butanediol(0.5 mol) | | | | | | |
| 2 | Diethyl terephthalate (1.0 | 11k | 22k | 1.9 | 139 | 10 | Almost soluble |
| | mol) | | | | | | |
| | 1,5-pentanediol (0.5 mol) | | | | | | |
| | 1,4-butanediol (0.5 mol) | | | | | | |
| 3 | Diethyl terephthalate (1.0 | 13k | 24k | 1.9 | 120 | 10 | Almost soluble |
| | mol) | | | | | | |
| | 1,5-pentanediol (0.6 mol) | | | | | | |
| | 1,4-butanediol(0.4 mol) | | | | | | |
| 4 | Diethyl terephthalate (1.0 | 8.6k | 16k | 1.9 | 101 | 4 | Almost soluble |
| | mol) | | | | | | |
| | 1,5-pentanediol (0.7 mol) | | | | | | |
| | 1,4-butanediol (0.3 mol) | | | | | | |

The effect of different diesters on the solubility of compounds of structure (III) was studied by polymerizing either di-tert-butyl terephthalate or diethyl terephthalate with 1,4-butanediol and 1,6-hexanediol and the results are summarized in Table 11. As shown in Table 11, the polymer formed from di-tert-butyl terephthalate is less soluble than the polymer formed from diethyl terephthalate.

### Example 15

TABLE 12
EFFECT OF SOLVENT ON PROPERTIES
OF COMPOUNDS OF STRUCTURE (III)

| | | | Mn | Mw | | Tm | Tg |
|---|---|---|---|---|---|---|---|
| # | Diester-Diols | Solvent | (g/mol) | (g/mol) | PDI | (° C.) | (° C.) |
| 1 | Diethyl terephthalate (1.0 mol) | No | 11k | 22k | 1.9 | 139 | 10 |
| | 1,5-pentanediol (0.5 mol) | | | | | | |
| | 1,4-butanediol (0.5 mol) | | | | | | |
| 2 | Diethyl terephthalate (1.0 mol) | paraffin wax | 1.2k | 3k | 2.4 | 54 | 27 |
| | 1,5-pentanediol (0.5 mol) | | | | | 120 | |
| | 1,4-butanediol (0.5 mol) | | | | | 167 | |

The effect of solvents on the properties of compounds of structure (III) was studied by polymerizing diethyl terephthalate with 1,4-butanediol and 1,5-hexanediol without any solvent or using paraffin wax as a solvent and the results are summarized in Table 12. As shown in Table 12, using paraffin wax as a solvent increases the molecular weight of the resulting polymer.

### Example 16

TABLE 13
EFFECT OF THIRD DIOL ON PROPERTIES OF COMPOUNDS OF STRUCTURE (III)

| | | Mn | Mw | | Tm |
|---|---|---|---|---|---|
| # | Diester-Diols | ( g/mol) | (g/mol) | PDI | (° C.) |
| 1 | Di-tert-butyl terephthalate (1 mol) | 15 k | 30 k | 2.0 | 101 |
| | Bis(4-hydroxy butyl terephthalate) (0.3 mol) | | | | |
| | Bis(5-hydroxy pentyl terephthalate) (0.7 mol) | | | | |
| 2 | Di-tert-butyl terephthalate (1 mol) | 16 k | 31 k | 1.9 | 103 |
| | Bis(4-hydroxy butyl) terephthalate (0.295 mol) | | | | |
| | Bis(5-hydroxy pentyl) terephthalate (0.675 mol) | | | | |
| | Glycerol (crosslinker) (0.03 mol) | | | | |
| 3 | Di-tert-butyl terephthalate (1.0 mol) | 11 k | 29 k | 2.6 | 116 |
| | Bis(4-hydroxy butyl) terephthalate (0.295 mol) | | | | |
| | Bis(5-hydroxy pentyl) terephthalate (0.675 mol) | | | | |
| | Trimethyl-1,3,5-benzenetricarboxylate (crosslinker) (0.03 mol) | | | | |
| 4 | Di-tert-butyl terephthalate (1.0 mol) | 7.5 k | 16 k | 2.2 | 115 |
| | Bis(4-hydroxy butyl) terephthalate (0.24 mol) | | | | |
| | Bis(5-hydroxy pentyl) terephthalate (0.56 mol) | | | | |
| | 3-methyl-1,5-pentanediol terephthalate (0.2 mol) | | | | |
| 5 | Di-tert-butyl terephthalate = (1.6458 g; 1.0 mol) | 11.8 k | 35 k | 2.9 | 90 |
| | Bis(4-hydroxy butyl) terephthalate (0.5231 g; 0.24 m) | | | | |
| | Bis(5-hydroxy pentyl) terephthalate (1.3301 g; 0.56 m) | | | | |
| | Di[2-(2-hydroxyethylthio)ethyl] terephthalate (0.2 mol) | | | | |

The effect of a third diol monomer on the crystallinity of compounds of structure (III) was studied, and the results are summarized in Table 13. In this study, 3-methyl-1,5-pentanediol terephthalate or di[2-(2-hydroxyethylthio)ethyl]terephthalate was used as the third diol monomer, alongside bis(4-hydroxy butyl terephthalate) and bis(5-hydroxy pentyl terephthalate), to polymerize with di-tert-butyl terephthalate. The polymer obtained using di[2-(2-hydroxyethylthio)ethyl]terephthalate as the third monomer exhibits a lower melting temperature (Tm) compared to the polymer derived from the polymerization of di-tert-butyl terephthalate and bis(4-hydroxy butyl terephthalate) and bis(5-hydroxy pentyl terephthalate. The result indicates that incorporating a third monomer can further reduce the crystallinity content of the polymer.

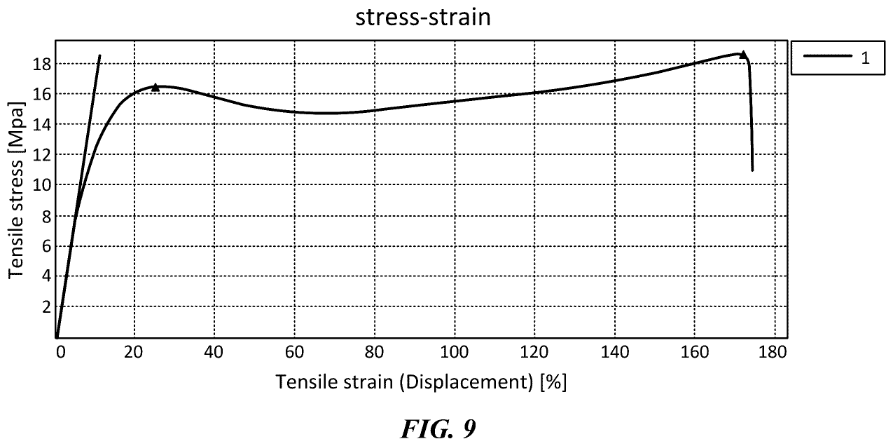

The effect of relative amounts of bis(4-hydroxy butyl) terephthalate and bis(4-hydroxy pentyl) terephthalate monomers on the crystallinity of compounds of structure (III) was studied. As shown in FIG. 10, initially, as the bis(4-hydroxy butyl) terephthalate content increases, the crystalline content decreases while transparency increases, reaching maximum transparency and minimum crystallinity at about 35 mol %. Beyond this point, further increases in bis(4-hydroxy butyl) terephthalate lead to a rise in crystalline content and a corresponding decrease in transparency.

### Example 17

TABLE 14
EFFECT OF DIOL RATIOS ON PROPERTIES OF COMPOUNDS OF STRUCTURE (III)

| | | Mn | Mw | | |
|---|---|---|---|---|---|
| # | Diester-Diols | (g/mol) | (g/mol) | PDI | Tm (° C.) |
| 1 | Di-tert-butyl terephthalate (1.0 mol) | 9 k | 17 k | 1.9 | 94 |
| | Bis(5-hydroxy pentyl) terephthalate (0.7 mol) | | | | |
| | Bis(6-hydroxy hexyl) terephthalate (0.3 mol) | | | | |
| 2 | Di-tert-butyl terephthalate (1.0 mol) | 9 k | 17 k | 1.9 | 77 |
| | Bis(5-hydroxy pentyl) terephthalate (0.6 mol) | | | | |
| | Bis(6-hydroxy hexyl) terephthalate (0.4 mol) | | | | |
| 3 | Di-tert-butyl terephthalate (1.0 mol) | 15 k | 27 k | 1.7 | 86 |
| | Bis(5-hydroxy pentyl) terephthalate (0.5 mol) | | | | |
| | Bis(6-hydroxy hexyl) terephthalate (0.5 mol) | | | | |
| 4 | Di-tert-butyl terephthalate (1.0 mol) | 11 k | 24 k | 1.2 | 103 |
| | Bis(5-hydroxy pentyl) terephthalate (0.4 mol) | | | | |
| | Bis(6-hydroxy hexyl) terephthalate (0.6 mol) | | | | |
| 5 | Di-tert-butyl terephthalate (1.0 mol) | 13 k | 27 k | 2.0 | 116 |
| | Bis(5-hydroxy pentyl) terephthalate (0.3 mol) | | | | |
| | Bis(6-hydroxy hexyl) terephthalate (0.7 mol) | | | | |

The effect of relative amounts of two diol monomers on Tm and crystallinity of compounds of structure (III) was studied, and the results are summarized in Table 14. In this study, polymers were synthesized through the polymerization of di-tert-butyl terephthalate with bis(5-hydroxy pentyl) terephthalate and bis(6-hydroxy hexyl) terephthalate. The ratio of bis(5-hydroxy pentyl) terephthalate to bis(6-hydroxy hexyl) terephthalate was varied from 7:3, 3:2:1:1, 2:3, to 3:7.

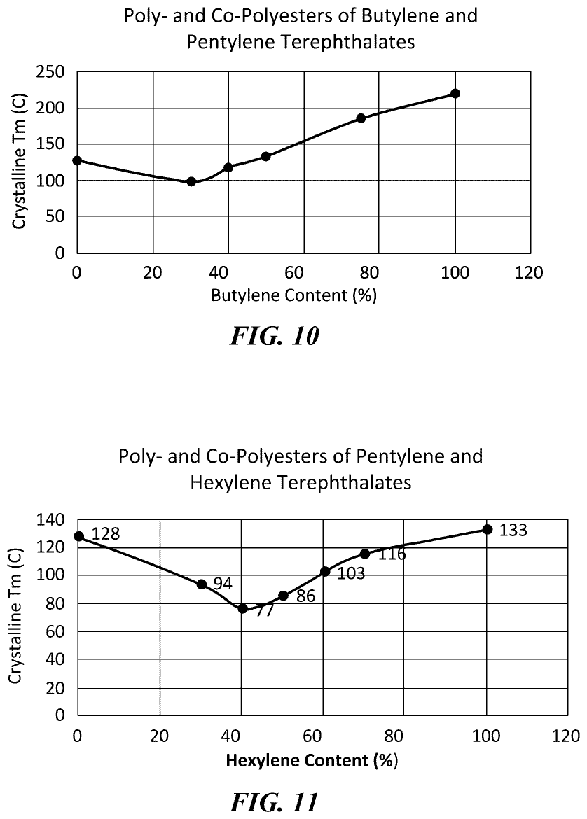

As shown in Table 14, the melting point (Tm) of the polymer initially decreases with increasing amounts of bis(6-hydroxy hexyl) terephthalate, but begins to rise once the proportion exceeds 40 mol % relative to bis(5-hydroxy pentyl) terephthalate. FIG. 11 illustrates how the crystalline content and transparency are affected. Initially, as the bis(6-hydroxy hexyl) terephthalate content increases, the crystalline content decreases while transparency increases, reaching maximum transparency and minimum crystallinity at 40 mol %. Beyond this point, further increases in bis(6-hydroxy hexyl) terephthalate lead to a rise in crystalline content and a corresponding decrease in transparency.

### Example 18

### Treatment Using an Orthodontic Appliance

This example describes the use of a directly 3D printed orthodontic appliance to move a patient's teeth according to a treatment plan. This example also describes the characteristics that the orthodontic appliance can have following its use, in contrast to its characteristics prior to use.

A patient in need of, or desirous of, a therapeutic treatment to rearrange at least one tooth has their teeth arrangement assessed. An orthodontic treatment plan is generated for the patient. The orthodontic treatment plan comprises a plurality of intermediate tooth arrangements for moving teeth along a treatment path, from the initial arrangement (e.g., that which was initially assessed) toward a final arrangement. The treatment plan includes the use of an orthodontic appliance, fabricated using curable composition s and methods disclosed further herein, to provide orthodontic appliances having low levels of hydrogen bonding units. In some embodiments, a plurality of orthodontic appliances is used, each of which can be fabricated using the curable composition comprising one or more polymerizable compounds and methods disclosed further herein.

The orthodontic appliances are provided, and iteratively applied to the patient's teeth to move the teeth through each of the intermediate tooth arrangements toward the final arrangement. The patient's tooth movement is tracked. A comparison is made between the patient's actual teeth arrangement and the planned intermediate arrangement. Where the patient's teeth are determined to be tracking according to the treatment plan, but have not yet reached the final arrangement, the next set of appliances can be administered to the patient. The threshold difference values of a planned position of teeth to actual positions selected as indicating that a patient's teeth have progressed on-track are provided above in Table 3. If a patient's teeth have progressed at or within the threshold values, the progress is considered to be on-track. Favorably, the use of the appliances disclosed herein increases the probability of on-track tooth movement.

The assessment and determination of whether treatment is on-track can be conducted, for example, 1 week (7 days) following the initial application of an orthodontic appliance. Following this period of application, additional parameters relating to assessing the durability of the orthodontic appliance can also be conducted. For example, relative repositioning force (compared to that which was initially provided by the appliance), remaining flexural stress, relative flexural modulus, and relative elongation at break can be determined.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A polymerizable compound having the following structure (III): 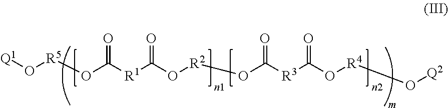
wherein:
R¹ and R³ are, at each occurrence, independently an arylene or heteroarylene group;
R² is, at each occurrence, independently a linear alkylene or arylene bis(alkylene ester) group;
R⁴ is, at each occurrence, independently a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
R⁵ is a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group;
Q¹ and Q² are each independently an end-capping moiety comprising one or more reactive functional groups;
m is an integer of one or greater; and
n1 and n2 are, at each occurrence, independently an integer of one or greater for each integral value of m,
provided that if R¹ and R³ are the same, then R² and R⁴ are different, and if R² and R⁴ are the same, then R¹ and R³ are different.

2. (canceled)

3. The compound of claim 1, wherein R¹ and R³ are phenylene, biphenylene, naphthalene, furanylene, thienylene, pyrrolyene, imidazolyene, pyrazolyene, oxazolyene or thiazolyen, wherein the phenylene, biphenylene or naphthalene is optionally substituted with one or more C₁-C₆ alkyl or C₁-C₆ alkoxy.

4. (canceled)

5. The compound of claim 1, wherein R¹ and R³ have one of the following structures: 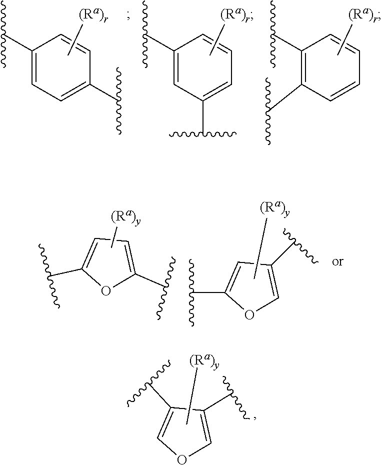
wherein:
R^{a} is, at each occurrence, C₁-C₆ alkyl or C₁-C₆ alkoxy;
r is an integer from 0 to 4; and
y is an integer from 0 to 2.

6. The compound of claim 5, wherein R¹ and R³ have one of the following structures:

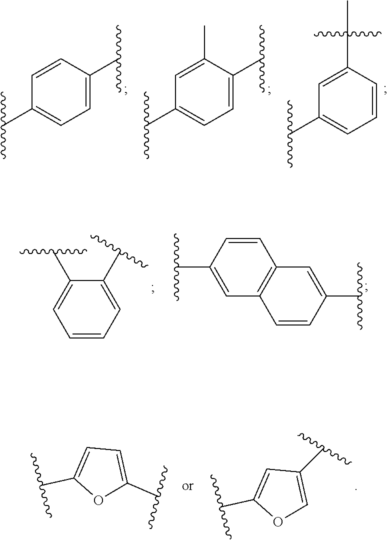

7. The compound of claim 1, wherein R¹ is an unsubstituted 1,4-phenylene group and R³ is a substituted or unsubstituted phenylene group, and the compound has the following structure (IA): 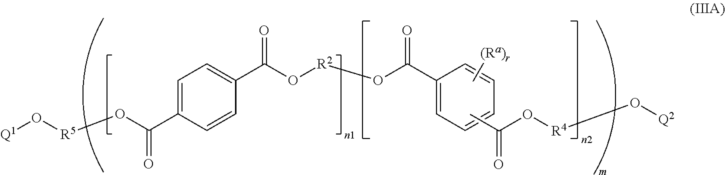
wherein:
R^{a} is, at each occurrence, C₁-C₆ alkyl or C₁-C₆ alkoxy; and
r is, at each occurrence, an integer from 0 to 4,
provided that if r is 0 and both phenylenes are unsubstituted 1,4-phenylene groups, then R² and R⁴ are different.

8. The compound of claim 7, wherein R³ is a substituted or unsubstituted 1,4-phenylene group, and the compound has the following structure (IIIB):

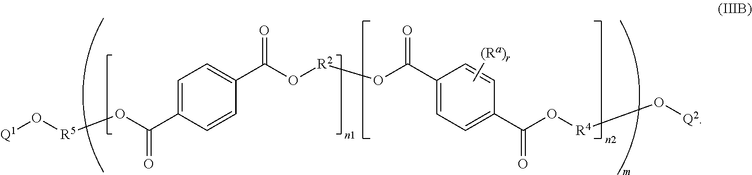

9. The compound of claim 8, wherein R³ is an unsubstituted 1,2-phenylene or 1,3-phenylene group, and the compound has the following structure (IIIC) or (IIID):

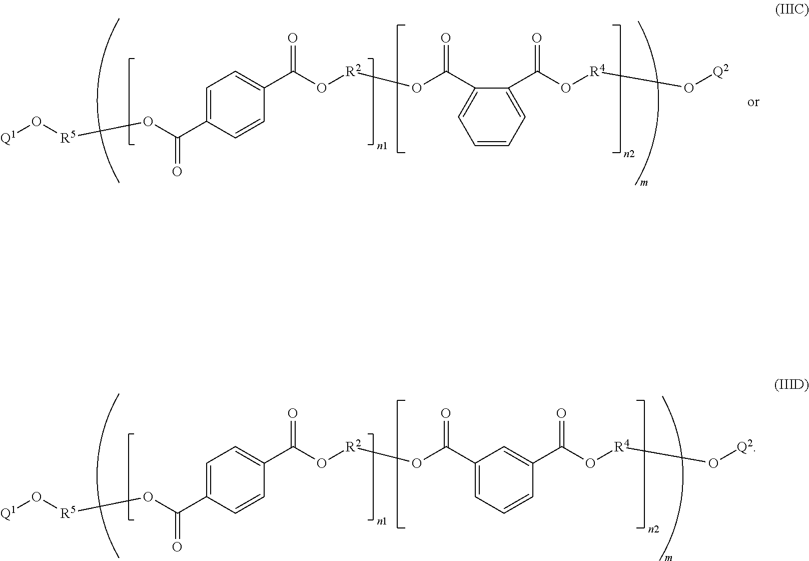

10. The compound of claim 1, wherein R⁴ is a linear or branched C₁-C₁₂ alkylene.

11. The compound of claim 10, wherein R⁴ is ethylene, propylene, isopropylene, n-butylene, isobutylene, tert-butylene, pentylene, isopentylene, 3-methylpentylene, hexylene, octylene, nonylene, decylene or dodecylene.

12. The compound of claim 1, wherein R⁴ is a linear or branched C₂-C₁₂ heteroalkylene.

13. The compound of claim 12, wherein R⁴ has one of the following structures: 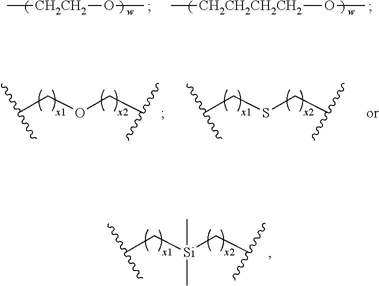
wherein:
w is an integer from 1 to 500; and
x1 and x2 are independently an integer from 1 to 6.

14. The compound of claim 1, wherein R⁴ is a phenylene bis(alkylene ester) or phenylene bis(heteroalkylene ester)group, and the compound has the following structure (IIIE): 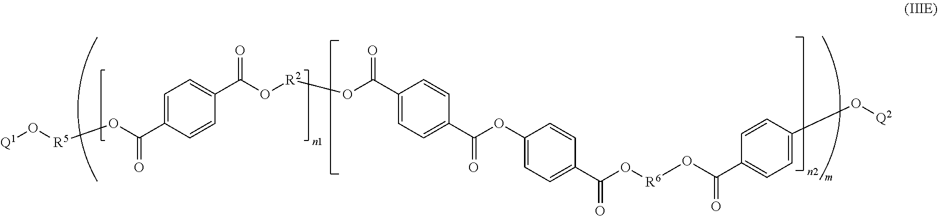
wherein:
R⁶ is, at each occurrence, a linear or branched C₁-C₁₂ alkylene, C₃-C₁₈ cycloalkylene or linear or branched C₂-C₁₂ heteroalkylene group.

15. The compound of claim 1, wherein R⁴ is a bis(carboxycyclohexylene) alkylene or bis(carboxycyclohexylene) heteroalkene group, and the compound has the following structure (IIIF): 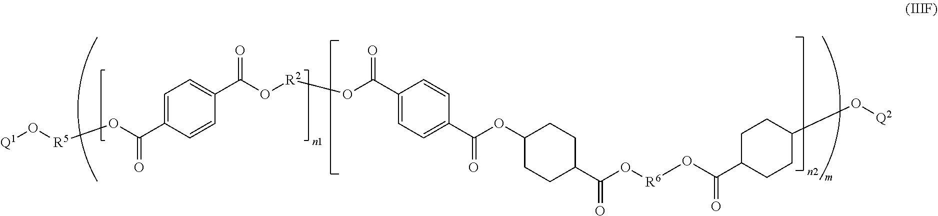
wherein:
R⁶ is, at each occurrence, a linear or branched C₁-C₁₂ alkylene, C₃-C₁₈ cycloalkylene, or linear or branched C₂-C₁₂ heteroalkylene group

16. The compound of claim 14, wherein R⁶ has one of the following structures:

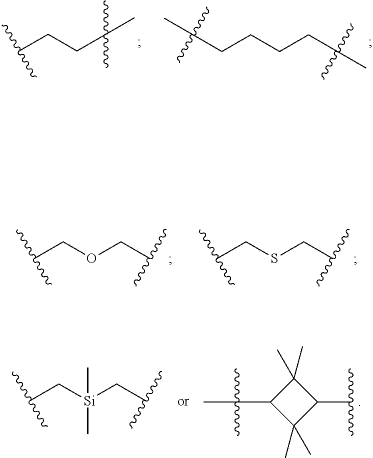

17. The compound of claim 1, wherein n1 and n2 are each independently an integer from 1 to 50, and wherein m is an integer from 1 to 50.

18. The compound of claim 1, wherein the compound has the following structure (IIIG): 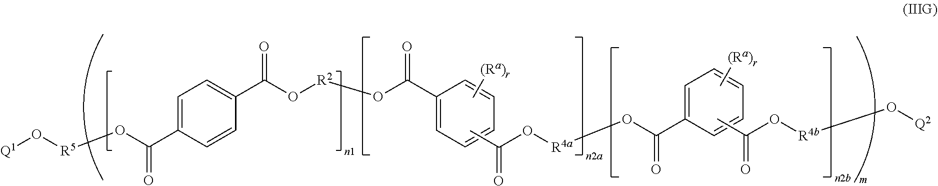
wherein:
R^{4a} and R^{4b}, at each occurrence, a linear or branched alkylene, linear or branched heteroalkylene, arylene bis(alkylene ester), arylene bis(heteroalkylene ester), bis(carboxyarylene) alkylene, bis(carboxyarylene) cylcoalkylene, bis(carboxyarylene) heteroalkylene, bis(carboxycycloalkylene) alkylene or bis(carboxycycloalkylene) heteroalkylene group, provided that R^{4a} and R^{4b} are different;
R^{a} is, at each occurrence, C₁-C₆ alkyl or C₁-C₆ alkoxy;
r is, at each occurrence, an integer from 0 to 4;
m is an integer of one or greater; and
n1, n2a and n2b are, at each occurrence, independently an integer of one or greater for each integral value of m.

19. The compound of claim 18, wherein R^{4a} and R^{4b} are each a linear or branched C₁-C₁₂ alkylene.

20. The compound of claim 19, wherein R^{4a} and R^{4b} are independently ethylene, propylene, isopropylene, n-butylene, isobutylene, tert-butylene, pentylene, isopentylene, 3-methylpentylene, hexylene, octylene, nonylene, decylene or dodecylene.

21. The compound of claim 18, wherein R^{4a} and R^{4b} are each a linear or branched C₂-C₁₂ heteroalkylene.

22. The compound of claim 21, wherein R^{4a} and R^{4b} independently have one of the following structures: 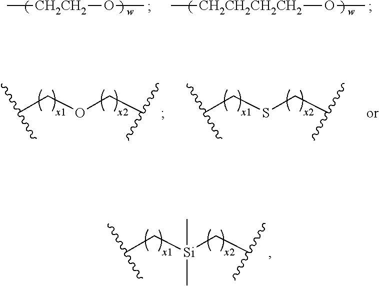
wherein:
w is an integer from 1 to 500; and
x1 and x2 are independently an integer from 1 to 6.

23. The compound of claim 18, wherein n1, n2a and n2b are each independently an integer from 1 to 50.

24. (canceled)

25. The compound of claim 1, wherein R² is ethylene, propylene, butylene, pentylene, hexylene, octylene, nonylene, decylene or dodecylene, and R⁵ is ethylene, propylene, butylene, pentylene or hexylene.

26.-28. (canceled)

29. The compound of claim 1, wherein Q¹ and Q² independently comprise an acrylate, methacrylate, vinyl acrylate, vinyl methacrylate, allyl ether, silene, alkyne, alkene, vinyl ether, maleimide, fumarate, maleate, itaconate, vinyl ester, vinyl ketone, epoxide, oxetane, cyclic ester or styrenyl moiety.

30. The compound of claim 29, wherein Q¹ and Q² independently have one of the following structures: 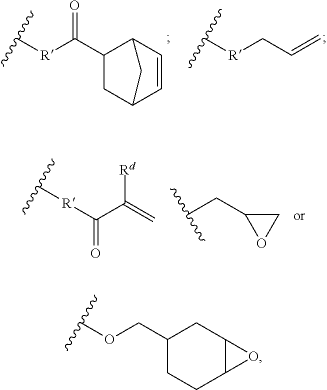
wherein:
R′ is an optional C₁-C₆ heteroalkylene linker; and
R^{d} is H, halogen, or C₁-C₃ alkyl.

31. The compound of claim 30, wherein Q¹ and Q² independently have one of the following structures:

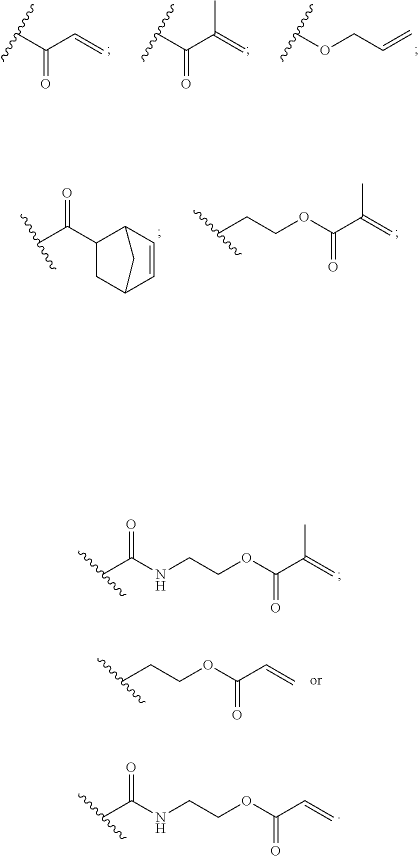

32. (canceled)

33. The compound of claim 1, wherein the compound of structure (III) is a compound having the following structures:

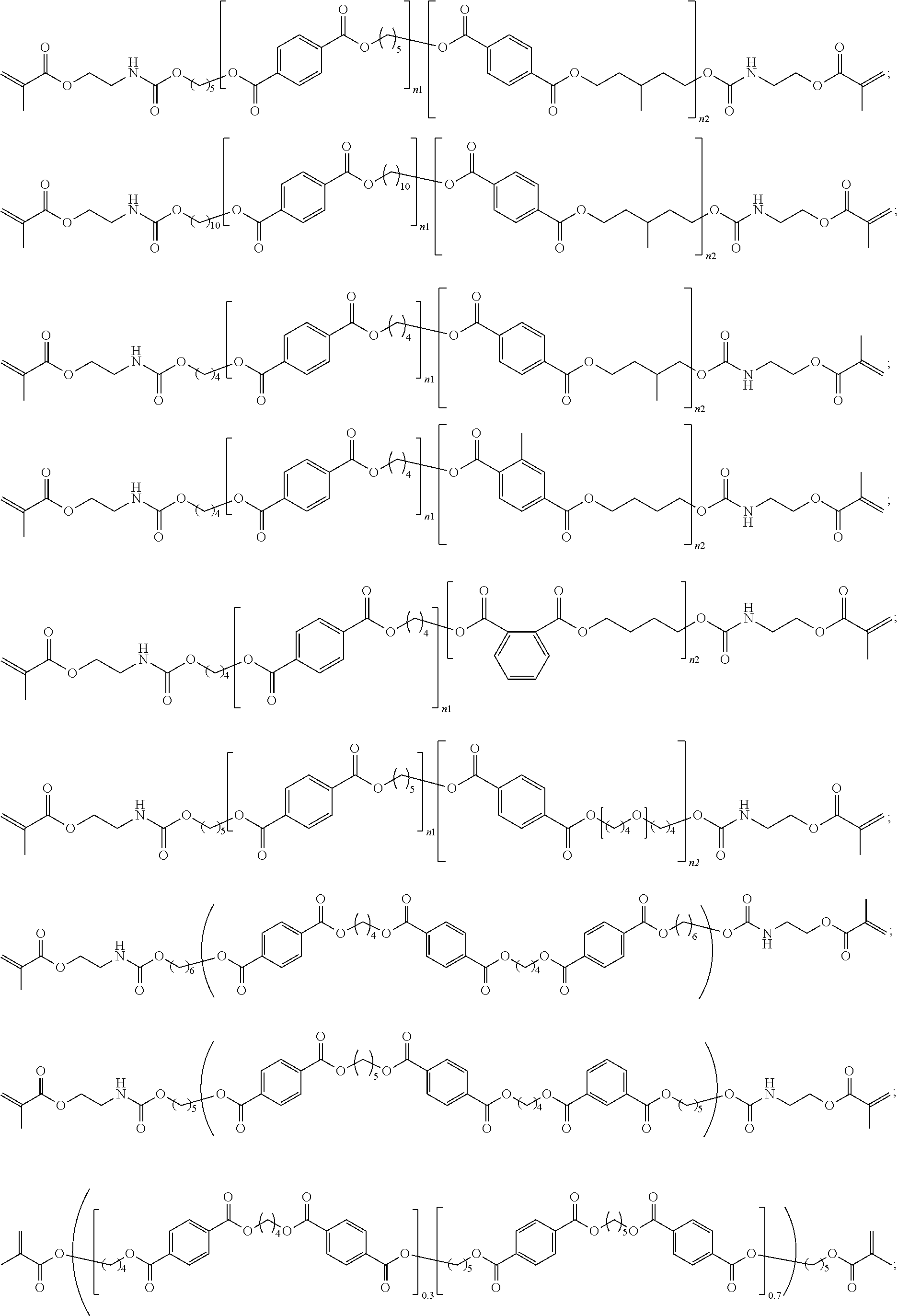

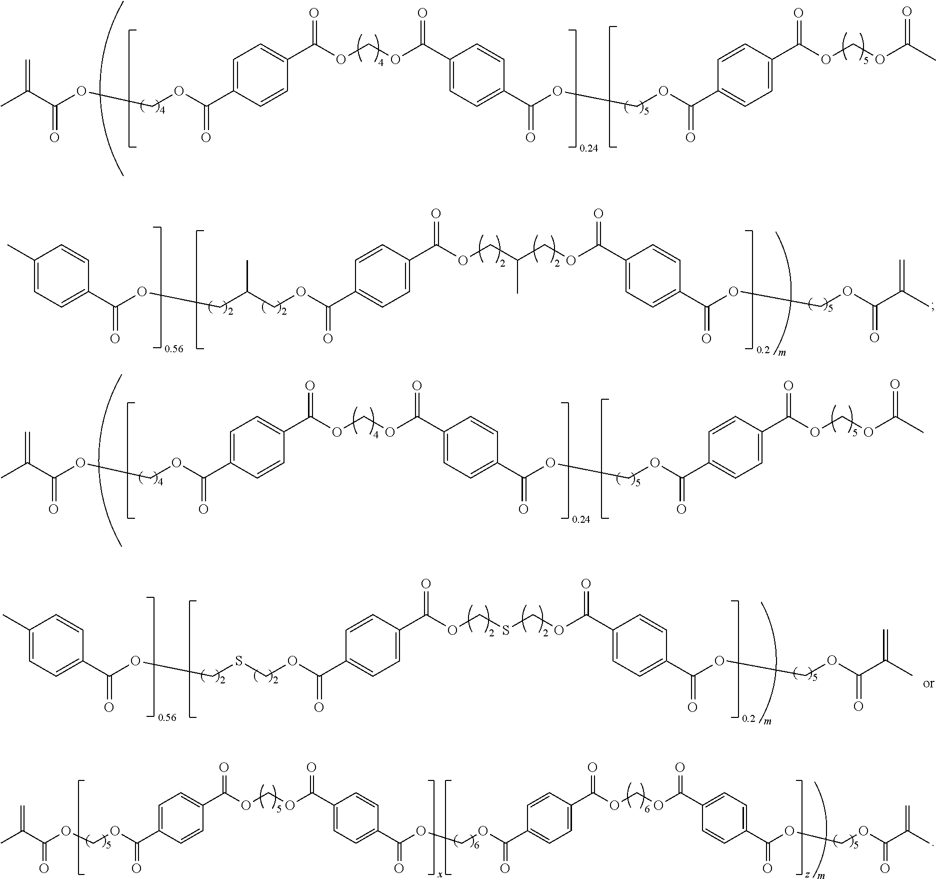

34. A curable composition, comprising:
an initiator; and
a polymerizable compound of claim 1.

35. A curable composition, comprising:
an initiator; and
a polymerizable compound having the following structure (II): 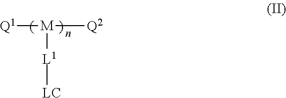
wherein:
M is an atom or group of atoms of a polymer backbone;
LC is a liquid crystalline moiety;
L¹ is an optional alkylene or heteroalkylene linker covalently bound a liquid crystalline moiety to the polymer backbone;
Q¹ and Q² are each independently an end-capping moiety comprising one or more reactive functional groups; and
n is an integer of one or greater.

36.-98. (canceled)

99. An orthodontic appliance comprising a polymeric material formed from the curable composition of claim 1.

100.-110. (canceled)

111. A method for preparing an article by an additive manufacturing process, comprising:
providing a curable composition of claim 34;
heating the curable composition to a processing temperature;
exposing the curable composition to radiation;
curing the curable composition layer-by-layer based on a predefined design, thereby
polymerizing and crosslinking the polymerizable compound to form a polymeric material; and
fabricating the article with the polymeric material.

112.-121. (canceled)
